(19) United States
(12) Patent Application Publication
(10) Pub. No.: US 2023/0303510 A1
(43) Pub. Date: Sep. 28, 2023

(54) CYCLIZED ACETAMIDO DERIVATIVES AS DNA POLYMERASE THETA INHIBITORS

(71) Applicant: Ideaya Biosciences, Inc., South San Francisco, CA (US)

(72) Inventors: Hilary Plake BECK, Soth San Francisco, CA (US); Michael Patrick DILLON, South San Francisco, CA (US); Brian Thomas JONES, South San Francisco, CA (US); Luisruben Padilla MARTINEZ, South San Francisco, CA (US)

(21) Appl. No.: 18/006,991

(22) PCT Filed: Jul. 28, 2021
(86) PCT No.: PCT/US2021/043482

§ 371 Date: Jan. 26, 2023

Related U.S. Application Data
(60) Provisional application No. 63/058,309, filed on Jul. 29, 2020.

(51) Int. Cl. C07D 401/04; C07D 207/09; C07D 413/04; C07D 401/14; C07D 405/14; C07D 403/04

(52) U.S. Cl. CPC C07D 401/04; C07D 207/09; C07D 413/04; C07D 401/14; C07D 405/14; C07D 403/04

(57) ABSTRACT

The present invention relates to methods for treating bacterial infections caused by or

[symbol]

Provided and set forth herein are certain cyclized acetamido derivatives that are DNA Polymerase Theta (Polθ) inhibitors of Formula (I) and Formula II.

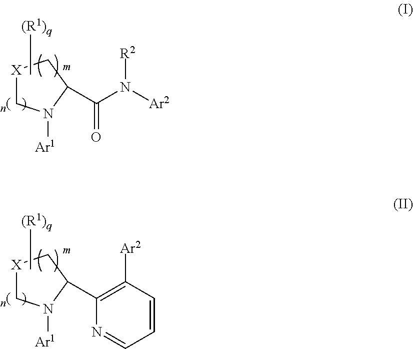

Also, provided are pharmaceutical compositions comprising such compounds, and methods of treating diseases treatable by inhibition of Polθ such as cancer, including homologous recombination (HR) deficient cancers, using such compounds and pharmaceutical compositions.

### CROSS-REFERENCE TO RELATED APPLICATIONS

This application claims priority to U.S. Provisional Application Ser. No. 63/058,309, filed Jul. 29, 2020, the disclosures of which are incorporated herein by reference in its entirety.

### REFERENCE TO A “SEQUENCE LISTING,” A TABLE, OR A COMPUTER PROGRAM LISTING APPENDIX SUBMITTED ON A COMPACT DISK

This application contains a Sequence Listing which has been submitted electronically in ASCII format and is hereby incorporated by reference in its entirety. Said ASCII copy, created on Jul. 27, 2021, is named LU67027_PCT_Seq_List_27jul.2021.txt and is 1,354 bytes in size.

### BACKGROUND

Targeting DNA repair deficiencies has become a proven and effective strategy in cancer treatment. However, DNA repair deficient cancers often become dependent on backup DNA repair pathways, which present an “Achilles heel” that can be targeted to eliminate cancer cells, and is the basis of synthetic lethality. Synthetic lethality is exemplified by the success of poly (ADP-ribose) polymerase (PARP) inhibitors in treating BRCA-deficient breast and ovarian cancers (Audeh M. W., et al., Lancet (2010); 376 (9737): 245-51).

DNA damage repair processes are critical for genome maintenance and stability, among which, double strand breaks (DSBs) are predominantly repaired by the nonhomologous end joining (NHEJ) pathway in G1 phase of the cell cycle and by homologous recombination (HR) in S-G2 phases. A less addressed alternative end-joining (alt-EJ), also known as microhomology-mediated end-joining (MMEJ) pathway, is commonly considered as a “backup” DSB repair pathway when NHEJ or HR are compromised. Numerous genetic studies have highlighted a role for polymerase theta (Polθ, encoded by POLQ) in stimulating MMEJ in higher organisms (see Chan S. H., et al., PLoS Genet. (2010); 6: e1001005; Roerink S. F., et al., Genome research. (2014); 24: 954-962; Ceccaldi R., et. al., Nature (2015); 518: 258-62; and Mateos-Gomez P. A., et al., Nature (2015); 518: 254-57).

The identification of mammalian POLQ initially arose from interest in the POLQ ortholog Mus308 gene product of Drosophila melanogaster. Mus308 mutants are hypersensitive to agents that cause DNA inter-strand cross-links (ICL) (Aguirrezabalaga I., et al., Genetics. (1995); 139:649-658), which implied that Mus308 may play a specific role in repair of ICLs in DNA. Characterization of the POLQ gene showed that it encodes an unusual domain configuration, with a large central portion flanking by a N-terminal DNA helicase domain and a C-terminal DNA polymerase domain (see Harris P. V., et al., Mol Cell Biol. (1996); 16: 5764-5771). The mechanisms by which Polθ polymerase functions in alt-EJ were also found to efficiently promote end-joining when overhangs contained >2 bp of microhomology were present (see Kent T., et al., Elife (2016); 5: e13740), and Kent T., et al., Nat. Struct. Mol. Biol. (2015); 22: 230-237. On the other hand, the helicase domain of Polθ contributes to microhomology annealing (see Chan S H et al., PLoS Genet. (2010); 6: e1001005; and Kawamura K et al., Int. J. Cancer (2004); 109: 9-16).

The expression of Polθ is largely absent in normal cells but upregulated in breast, lung, and ovarian cancers (see Ceccaldi R., et al., Nature (2015); 518, 258-62). Additionally, the increase of Polθ expression correlates with poor prognosis in breast cancer (see Lemee F et al., Proc Natl Acad Sci USA. (2010); 107: 13390-5). It has been shown that cancer cells with deficiency in HR, NHEJ or ATM are highly dependent on Polθ expression (see Ceccaldi R., et al., Nature (2015); 518: 258-62, Mateos-Gomez P A et al., Nature (2015); 518: 254-57, and Wyatt D. W., et al., Mol. Cell (2016); 63: 662-73). Therefore, Polθ is an attractive target for novel synthetic lethal therapy in cancers containing DNA repair defects.

### SUMMARY

According to the inventive concept, provided herein are acetamido derivatives that are DNA Polymerase Theta (Polθ) inhibitors, and in some aspects, compounds that inhibit the polynerase domain of Polθ. Also, provided herein are pharmaceutical compositions including such compounds and methods of treating and/or preventing diseases treatable by inhibition of Polθ such as cancer, including homologous recombination (HR) deficient cancers.

In a first aspect of the inventive concept, provided herein are compounds of Formula (I) and Formula (II):

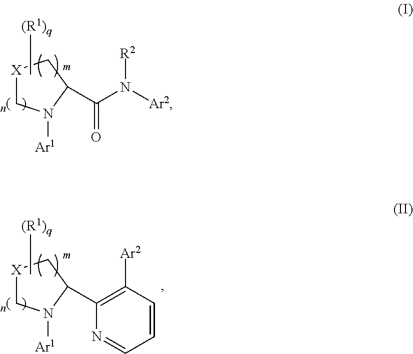

or a pharmaceutically acceptable salt thereof, wherein
X is selected from the group consisting of —CH₂—, —CHR¹—, —NR¹—, —NH—, and —O—;
m is an integer selected from the group consisting of 0, 1, and 2;
n is an integer selected from the group consisting of 0, 1, and 2;
provided that the sum of m and n is at least 1 and no more than 3;
q is an integer selected from the group consisting of 0, 1, and 2;
each R¹ is independently selected from the group consisting of C₁₋₈ alkyl, C₁₋₈ haloalkyl, —OR^{a},
   —X¹—OR^{a}, —NR^{a}R^{b}, —X¹—NR^{a}R^{b}—NR^{a}C(O)R^{b}, —X¹—NR^{a}C(O)R^{b}, —C(O)NR^{a}R^{b}, —X¹—C(O)NR^{a}R^{b}, —C(O)R^{a}, —X¹—C(O)R^{a}, phenyl, and —X¹-phenyl, wherein
   X¹ is C₁₋₃ alkylene;
   each R^{a} and R^{b} are independently selected from the group consisting of H, C₁₋₄ alkyl, and C₁₋₄ haloalkyl; and
   phenyl is substituted with from 0 to 3 R^{c} moieties, each R^{c} is selected from the group consisting of C₁₋₈ alkyl, halo, C₁₋₈ haloalkyl, C₁₋₈ alkoxy, C₁₋₈ haloalkoxy, —OH,
   —X^{c}—OH and cyano, wherein X^{c} is C₁₋₃ alkylene;
Ar¹ is selected from the group consisting of phenyl and 6- to 10-membered heteroaryl having 1 to 4 heteroatom ring vertices independently selected from the group consisting of N, O, and S, wherein
   Ar¹ is substituted with 0 to 4 R^{d} moieties, wherein each R^{d} is independently selected from the group consisting of C₁₋₈ alkyl, halo, C₁₋₈ haloalkyl, cyano, —OR^{e}, —NR^{e}R^{f},
      —NR^{e}C(O)R^{f}, and —C(O)NR^{e}R^{f}, wherein each R^{e} and R^{f} are independently selected from the group consisting of H, C₁₋₆ alkyl, and C₁₋₆ haloalkyl;
R², when present, is selected from the group consisting of C₁₋₈ alkyl, C₁₋₈ haloalkyl, C₃₋₆ cycloalkyl, phenyl, and 3- to 6-membered heterocycloalkyl having 1 to 3 heteroatom ring vertices independently selected from the group consisting of N, O, and S; and
Ar² is selected from the group consisting of phenyl and 6- to 10-membered heteroaryl having 1 to 4 heteroatom ring vertices independently selected from the group consisting of N, O, and S, wherein
   Ar² is substituted with 0 to 4 R^{h} moieties, wherein each R^{h} is independently selected from the group consisting of C₁₋₈ alkyl, C₁₋₈ haloalkyl, halo, cyano, C₃₋₆ cycloalkyl,
      —ORⁱ, —NR^{j}R^{k}, —NR^{j}C(O)R^{k}, and —C(O)NR^{j}R^{k}, wherein
   each Rⁱ is selected from the group consisting of H, C₁₋₆ alkyl, C₁₋₆ haloalkyl, and C₃₋₆ cycloalkyl; and
each R^{j} and R^{k} are independently selected from the group consisting of H, C₁₋₆ alkyl, and C₁₋₆ haloalkyl.

In embodiments of the compound of Formula (I) provided by the first aspect of the inventive concept, if m=1, n=1, and the compound has a Formula (Ia-2)

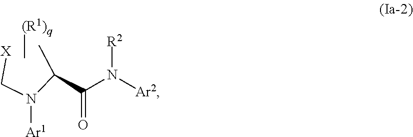

then Ar¹ is not 2-pyridyl, or 2-pyrimidinyl, or Ar² is not phenyl or 2-pyridyl.

In another aspect of the inventive concept, provided is compound of Formula (I):

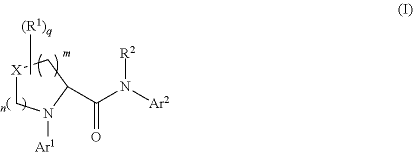

or a pharmaceutically acceptable salt thereof, wherein
X is selected from the group consisting of —CH₂—, —CHR¹—, —NR¹—, —NH—, and —O—;
m is an integer selected from the group consisting of 0, 1, and 2;
n is an integer selected from the group consisting of 0, 1, and 2;
provided that the sum of m and n is at least 1 and no more than 3;
q is an integer selected from the group consisting of 0, 1, and 2;
each R¹ is independently selected from the group consisting of C₁₋₈ alkyl, C₁₋₈ haloalkyl, —OR^{a},
   —X¹—OR^{a}, —NR^{a}R^{b}, —X¹—NR^{a}R^{b}, NR^{a}C(O)R^{b}, —X¹—NR^{a}C(O)R^{b}, —C(O)NR^{a}R^{b}, —X¹—C(O)NR^{a}R^{b}, —C(O)R^{a}, —X¹—C(O)R^{a}, phenyl, and —X¹-phenyl, wherein
   X¹ is C₁₋₃ alkylene;
   each R^{a} and R^{b} are independently selected from the group consisting of H, C₁₋₄ alkyl, and C₁₋₄ haloalkyl; and
   phenyl is substituted with from 0 to 3 R^{c} moieties, each R^{c} is selected from the group consisting of C₁₋₈ alkyl, halo, C₁₋₈ haloalkyl, C₁₋₈ alkoxy, C₁₋₈ haloalkoxy, —OH,
   —X^{c}—OH, and cyano, wherein X^{c} is C₁₋₃ alkylene;
Ar¹ is selected from the group consisting of phenyl and 6- to 10-membered heteroaryl having 1 to 4 heteroatom ring vertices independently selected from the group consisting of N, O, and S, wherein
   Ar¹ is substituted with 0 to 4 R^{d} moieties, wherein each R^{d} is independently selected from the group consisting of C₁₋₈ alkyl, halo, C₁₋₈ haloalkyl, cyano, —OR^{e}, —NR^{e}R^{f},
      —NR^{e}C(O)R^{f}, and —C(O)NR^{e}R^{f}, wherein
      each R^{e} and R^{f} are independently selected from the group consisting of H, C₁₋₆ alkyl, and C₁₋₆ haloalkyl;
R² is selected from the group consisting of C₁₋₈ alkyl, C₁₋₈ haloalkyl, C₃₋₆ cycloalkyl, phenyl, and 3- to 6-membered heterocycloalkyl having 1 to 3 heteroatom ring vertices independently selected from the group consisting of N, O, and S; and
Ar² is selected from the group consisting of phenyl and 6- to 10-membered heteroaryl having 1 to 4 heteroatom ring vertices independently selected from the group consisting of N, O, and S, wherein
   Ar² is substituted with 0 to 4 R^{h} moieties, wherein each R^{h} is independently selected from the group consisting of C₁₋₈ alkyl, C₁₋₈ haloalkyl, halo, cyano, C₃₋₆ cycloalkyl,
      —ORⁱ, —NR^{j}R^{k}, —NR^{j}C(O)R^{k}, and —C(O)NR^{j}R^{k}, wherein
   each Rⁱ is selected from the group consisting of H, C₁₋₆ alkyl, C₁₋₆ haloalkyl, and C₃₋₆ cycloalkyl; and
   each R^{j} and R^{k} are independently selected from the group consisting of H, C₁₋₆ alkyl, and C₁₋₆ haloalkyl, provided that
   if m=1, n=1, the compound has a Formula (Ia-2)

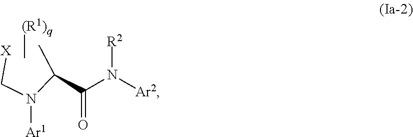

and
   if Ar¹ is 2-pyridyl or 2-pyrimidinyl, then Ar¹ is substituted with 1 to 4 R^{d} moieties, wherein each R^{d} is independently —NR^{e}C(O)R^{f}, or —C(O)NR^{e}R^{f}, or
   if Ar² is phenyl or 2-pyridyl, then Ar² is substituted with 1 to 4 R^{h} moieties wherein each R^{h} is independently —NR^{j}C(O)R^{k}, or —C(O)NR^{j}R^{k}.

In another aspect of the inventive concept, provided is compound of Formula (I):

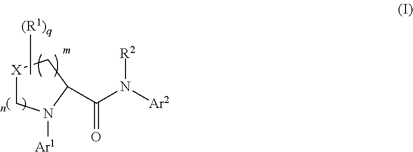

or a pharmaceutically acceptable salt thereof, wherein
X is selected from the group consisting of —CH₂—, —CHR¹—, —NR¹—, —NH—, and —O—;
m is an integer selected from the group consisting of 0, 1, and 2;
n is an integer selected from the group consisting of 0, 1, and 2;
provided that the sum of m and n is at least 1 and no more than 3;
q is an integer selected from the group consisting of 0, 1, and 2;
each R¹ is independently selected from the group consisting of C₁₋₈ alkyl, C₁₋₈ haloalkyl, —OR^{a},
   —X¹—OR^{a}, —NR^{a}R^{b}, —X¹—NR^{a}R^{b}—NR^{a}C(O)R^{b}, —X¹—NR^{a}C(O)R^{b}, —C(O)NR^{a}R^{b},
   —X¹—C(O)NR^{a}R^{b}, —C(O)R^{a}, —X¹—C(O)R^{a}, phenyl, and —X¹-phenyl, wherein X¹ is C₁₋₃ alkylene;
   each R^{a} and R^{b} are independently selected from the group consisting of H, C₁₋₄ alkyl, and C₁₋₄ haloalkyl; and
   phenyl is substituted with from 0 to 3 R^{c} moieties, each R^{c} is selected from the group consisting of C₁₋₈ alkyl, halo, C₁₋₈ haloalkyl, C₁₋₈ alkoxy, C₁₋₈ haloalkoxy, —OH,
      —X^{e}—OH, and cyano, wherein X^{e} is C₁₋₃ alkylene;
Ar¹ is selected from the group consisting of phenyl and 6- to 10-membered heteroaryl having 1 to 4 heteroatom ring vertices independently selected from the group consisting of N, O, and S, wherein
   Ar¹ is substituted with 0 to 4 R^{d} moieties, wherein each R^{d} is independently selected from the group consisting of C₁₋₈ alkyl, halo, C₁₋₈ haloalkyl, cyano, —OR^{e}, —NR^{e}R^{f},
      —NR^{e}C(O)R^{f}, and —C(O)NR^{e}R^{f}, wherein
      each R^{e} and R^{f} are independently selected from the group consisting of H, C₁₋₆ alkyl, and C₁₋₆ haloalkyl;
R² is selected from the group consisting of C₁₋₈ alkyl, C₁₋₈ haloalkyl, C₃₋₆ cycloalkyl, phenyl, and 3- to 6-membered heterocycloalkyl having 1 to 3 heteroatom ring vertices independently selected from the group consisting of N, O, and S; and
Ar² is selected from the group consisting of phenyl and 6- to 10-membered heteroaryl having 1 to 4 heteroatom ring vertices independently selected from the group consisting of N, O, and S, wherein
   Ar² is substituted with 1 to 4 R^{h} moieties, wherein each R^{h} is independently selected from the group consisting of cyano, —ORⁱ, —NR^{j}R^{k}, —NR^{j}C(O)R^{k}, and —C(O)NR^{j}R^{k}, wherein
   each Rⁱ is selected from the group consisting of C₁₋₆ haloalkyl, and C₃₋₆ cycloalkyl; and
   each R^{j} and R^{k} are independently selected from the group consisting of H, C₁₋₆ alkyl, and C₁₋₆ haloalkyl.

In yet another aspect of the inventive concept, provided is compound of Formula (I):

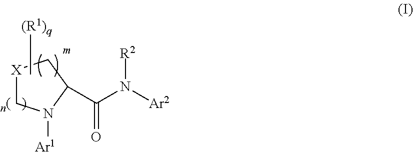

or a pharmaceutically acceptable salt thereof, wherein
X is selected from the group consisting of —CH₂—, —CHR¹—, —NR¹—, —NH—, and —O—;
m is an integer selected from the group consisting of 0, 1, and 2;
n is an integer selected from the group consisting of 0, 1, and 2;
provided that the sum of m and n is at least 1 and no more than 3;
q is an integer selected from the group consisting of 0, 1, and 2;
each Rⁱ is independently selected from the group consisting of C₁₋₈ alkyl, C₁₋₈ haloalkyl, —OR^{a},
   —X¹—OR^{a}, —NR^{a}R^{b}, —X¹—NR^{a}R^{b}—NR^{a}C(O)R^{b}, —X¹—NR^{a}C(O)R^{b}, —C(O)NR^{a}R^{b}, —X¹—C(O)NR^{a}R^{b}, —C(O)R^{a}, —X¹—C(O)R^{a}, phenyl, and —X¹-phenyl, wherein
   X¹ is C₁₋₃ alkylene;
   each R^{a} and R^{b} are independently selected from the group consisting of H, C₁₋₄ alkyl, and C₁₋₄ haloalkyl; and
   phenyl is substituted with from 0 to 3 R^{c} moieties, each R^{c} is selected from the group consisting of C₁₋₈ alkyl, halo, C₁₋₈ haloalkyl, C₁₋₈ alkoxy, C₁₋₈ haloalkoxy, —OH,
      —X^{c}—OH, and cyano, wherein X^{c} is C₁₋₃ alkylene;
Ar¹ is selected from the group consisting of phenyl and 6- to 10-membered heteroaryl having 1 to 4 heteroatom ring vertices independently selected from the group consisting of N, O, and S, wherein
   Ar¹ is substituted with 0 to 4 R^{d} moieties, wherein each R^{d} is independently selected from the group consisting of C₁₋₈ alkyl, halo, C₁₋₈ haloalkyl, cyano, —OR^{e}, —NR^{e}R^{f},
      —NR^{e}C(O)R^{f}, and —C(O)NR^{e}R^{f}, wherein
      each R and R^{f} are independently selected from the group consisting of H, C₁₋₆ alkyl, and C₁₋₆ haloalkyl;
R² is selected from the group consisting of C₁₋₈ alkyl, C₁₋₈ haloalkyl, C₃₋₆ cycloalkyl, phenyl, and 3- to 6-membered heterocycloalkyl having 1 to 3 heteroatom ring vertices independently selected from the group consisting of N, O, and S; and
Ar² is selected from the group consisting of phenyl and 6- to 10-membered heteroaryl having 1 to 4 heteroatom ring vertices independently selected from the group consisting of N, O, and S, wherein
   Ar² is substituted with 0 to 4 R^{h} moieties, wherein each R^{h} is independently selected from the group consisting of C₁₋₈ alkyl, C₁₋₈ haloalkyl, halo, cyano, C₃₋₆ cycloalkyl,
      —ORⁱ, —NR^{j}R^{k}, —NR^{j}C(O)R^{k}, and —C(O)NR^{j}R^{k}, wherein
   each Rⁱ is selected from the group consisting of H, C₁₋₆ alkyl, C₁₋₆ haloalkyl, and C₃₋₆ cycloalkyl; and
   each R^{j} and R^{k} are independently selected from the group consisting of H, C₁₋₆ alkyl, and C₁₋₆ haloalkyl, provided that
   if m=1, n=1, and the compound has a Formula (Ia-2)

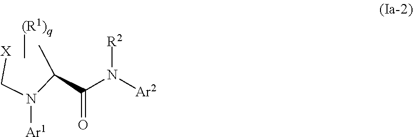

and Ar¹ is 2-pyridyl or 2-pyrimidinyl, then Ar² is substituted with 1 to 2 R^{h} moieties, wherein each R^{h} is cyano.

In yet another aspect of the inventive concept, provided is compound of Formula (I):

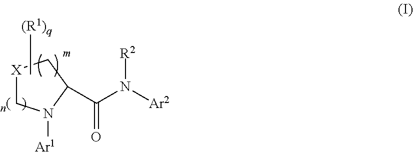

or a pharmaceutically acceptable salt thereof, wherein
X is selected from the group consisting of —CH₂—, —CHR¹—, —NR¹—, —NH—, and —O—;
m is an integer selected from the group consisting of 0, 1, and 2;
n is an integer selected from the group consisting of 0, 1, and 2;
provided that the sum of m and n is at least 1 and no more than 3;
q is an integer selected from the group consisting of 0, 1, and 2;
each R¹ is independently selected from the group consisting of —OR^{a}, and
   —X¹—OR^{a};
   wherein R^{a} is C₁₋₄ haloalkyl; and
   phenyl is substituted with from 0 to 3 R^{c} moieties, each R^{c} is selected from the group consisting of C₁₋₈ alkyl, halo, C₁₋₈ haloalkyl, C₁₋₈ alkoxy, C₁₋₈ haloalkoxy, —OH,
      —X^{c}—OH, and cyano, wherein X^{c} is C₁₋₃ alkylene;
Ar¹ is selected from the group consisting of phenyl and 6- to 10-membered heteroaryl having 1 to 4 heteroatom ring vertices independently selected from the group consisting of N, O, and S, wherein
   Ar¹ is substituted with 0 to 4 R^{d} moieties, wherein each R^{d} is independently selected from the group consisting of C₁₋₈ alkyl, halo, C₁₋₈ haloalkyl, cyano, —OR^{e}, —NR^{e}R^{f},
      —NR^{e}C(O)R^{f}, and —C(O)NR^{e}R^{f}, wherein
      each R and R^{f} are independently selected from the group consisting of H, C₁₋₆ alkyl, and C₁₋₆ haloalkyl;
R² is selected from the group consisting of phenyl, and 3- to 6-membered heterocycloalkyl having 1 to 3 heteroatom ring vertices independently selected from the group consisting of N, O, and S; and
Ar² is selected from the group consisting of phenyl and 6- to 10-membered heteroaryl having 1 to 4 heteroatom ring vertices independently selected from the group consisting of N, O, and S, wherein
   Ar² is substituted with 0 to 4 R^{h} moieties, wherein each R^{h} is independently selected from the group consisting of C₁₋₈ alkyl, C₁₋₈ haloalkyl, halo, cyano, C₃₋₆ cycloalkyl,
      —ORⁱ, —NR^{j}R^{k}, —NR^{j}C(O)R^{k}, and —C(O)NR^{j}R^{k}, wherein
   each Rⁱ is selected from the group consisting of H, C₁₋₆ alkyl, C₁₋₆ haloalkyl, and C₃₋₆ cycloalkyl; and
   each R^{j} and R^{k} are independently selected from the group consisting of H, C₁₋₆ alkyl, and C₁₋₆ haloalkyl.

In another aspect of the inventive concept, provided is compound of Formula (I):

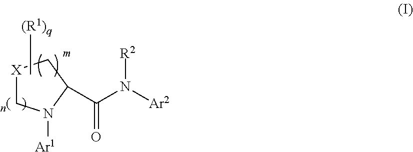

or a pharmaceutically acceptable salt thereof, wherein
X is selected from the group consisting of —CH₂—, —NH—, and —O—;
m is an integer selected from the group consisting of 0, 1, and 2;
n is an integer selected from the group consisting of 0, 1, and 2;
provided that the sum of m and n is at least 1 and no more than 3;
q is an integer selected from the group consisting of 0, 1, and 2;
each Rⁱ is independently selected from the group consisting of C₁₋₈ haloalkyl, —NR^{a}R^{b}, —X¹—NR^{a}R^{b}, —NR^{a}C(O)R^{b}, —X¹—NR^{a}C(O)R^{b}, —C(O)NR^{a}R^{b}, —X¹—C(O)NR^{a}R^{b}, —C(O)R^{a}, —X¹—C(O)R^{a}, phenyl, and —X¹-phenyl, wherein
   X¹ is C₁₋₃ alkylene;
   each R^{a} and R^{b} are independently selected from the group consisting of H, C₁₋₄ alkyl, and C₁₋₄ haloalkyl; and
   phenyl is substituted with from 0 to 3 R^{c} moieties, each R^{c} is selected from the group consisting of C₁₋₈ alkyl, halo, C₁₋₈ haloalkyl, C₁₋₈ alkoxy, C₁₋₈ haloalkoxy, —OH,
      —X^{c}—OH, and cyano, wherein X^{c} is C₁₋₃ alkylene;
Ar¹ is selected from the group consisting of phenyl and 6- to 10-membered heteroaryl having 1 to 4 heteroatom ring vertices independently selected from the group consisting of N, O, and S, wherein
   Ar¹ is substituted with 0 to 4 R^{d} moieties, wherein each R^{d} is independently selected from the group consisting of C₁₋₈ alkyl, halo, C₁₋₈ haloalkyl, cyano, —OR^{e}, —NR^{e}R^{f},
      —NR^{e}C(O)R^{f}, and —C(O)NR^{e}R^{f}, wherein
      each R^{e} and R^{f} are independently selected from the group consisting of H, C₁₋₆ alkyl, and C₁₋₆ haloalkyl;
R² is selected from the group consisting of C₁₋₈ alkyl, C₁₋₈ haloalkyl, C₃₋₆ cycloalkyl, phenyl, and 3- to 6-membered heterocycloalkyl having 1 to 3 heteroatom ring vertices independently selected from the group consisting of N, O, and S; and
Ar² is selected from the group consisting of phenyl and 6- to 10-membered heteroaryl having 1 to 4 heteroatom ring vertices independently selected from the group consisting of N, O, and S, wherein
   Ar² is substituted with 0 to 4 R^{h} moieties, wherein each R^{h} is independently selected from the group consisting of C₁₋₈ alkyl, C₁₋₈ haloalkyl, halo, cyano, C₃₋₆ cycloalkyl,
      —ORⁱ, —NR^{j}R^{k}, —NR^{j}C(O)R^{k}, and —C(O)NR^{j}R^{k}, wherein
   each Rⁱ is selected from the group consisting of H, C₁₋₆ alkyl, C₁₋₆ haloalkyl, and C₃₋₆ cycloalkyl; and
   each R^{j} and R^{k} are independently selected from the group consisting of H, C₁₋₆ alkyl, and C₁₋₆ haloalkyl.

In another aspect of the inventive concept, provided is a pharmaceutical composition including a compound of Formula (I), (II), a subembodiment as set forth herein, or a pharmaceutically acceptable salt thereof and at least one pharmaceutically acceptable excipient.

In yet another aspect of the inventive concept, provided are methods for treating and/or preventing a disease characterized by overexpression of Polθ in a patient including administering to the patient a therapeutically effective amount of a compound of Formula (I), (II), a subembodiment as set forth herein, or a pharmaceutically acceptable salt thereof, or a pharmaceutical composition including a compound of Formula (I) (or a subembodiment as set forth herein) and at least one pharmaceutically acceptable excipient.

In yet another aspect of the inventive concept, provided are methods for treating and/or preventing a disease characterized by overexpression of Polθ in a patient including administering to the patient a therapeutically effective amount of a compound of Formula (I), (II), a subembodiment as set forth herein, or a pharmaceutically acceptable salt thereof, or a pharmaceutical composition including a compound of Formula (I) (or a subembodiment as set forth herein) and at least one pharmaceutically acceptable excipient.

In yet another aspect of the inventive concept, provided are methods of treating and/or preventing a homologous recombinant (HR) deficient cancer in a patient including administering to the patient a therapeutically effective amount of a compound of Formula (I), (II), a subembodiment as set forth herein, or a pharmaceutically acceptable salt thereof.

In yet another aspect of the inventive concept, provided are methods for inhibiting DNA repair by Polθ in a cancer cell including contacting the cell with an effective amount of a compound of Formula (I), (II), a subembodiment as set forth herein, or a pharmaceutically acceptable salt thereof. In a first embodiment, the cancer is HR deficient cancer.

In yet a sixth aspect of the inventive concept, provided are methods for treating and/or preventing a cancer in a patient, wherein the cancer is characterized by a reduction or absence of BRCA gene expression, the absence of the BRCA gene, or reduced function of BRCA protein, including administering to the subject a therapeutically effective amount of a compound of Formula (I), (II), a subembodiment as set forth herein, or a pharmaceutically acceptable salt thereof optionally in a pharmaceutical composition.

In yet another aspect of the inventive concept, provided are compounds of Formula (I), (II), a subembodiment as set forth herein, or a pharmaceutically acceptable salt thereof for inhibiting DNA repair by Polθ in a cell. In an embodiment, the cell is HR deficient cell.

In yet another aspect of the inventive concept, provided are compounds of Formula (I), (II), a subembodiment as set forth herein, or a pharmaceutically acceptable salt thereof for use in the treatment and/or prevention of a disease in a patient, wherein the disease is characterized by overexpression of Pol.

In yet another aspect of the inventive concept, provided are compounds of Formula (I), (II), a subembodiment as set forth herein, or a pharmaceutically acceptable salt thereof for use in the treatment and/or prevention of a cancer in a patient, wherein the cancer is characterized by a reduction or absence of BRCA gene expression, the absence of the BRCA gene, or reduced function of BRCA protein.

In yet another aspect of the inventive concept, provided are compounds of Formula (I), (II), a subembodiment as set forth herein, or a pharmaceutically acceptable salt thereof for use in the treatment and/or prevention of a HR deficient cancer in a patient.

In yet another aspect of the inventive concept, provided are compounds of Formula (I), (II), a subembodiment as set forth herein, or a pharmaceutically acceptable salt thereof for use in the treatment or prevention of a cancer that is resistant to poly(ADP-ribose)polymerase (PARP) inhibitor therapy in a patient.

In yet another aspect of the inventive concept, provided are compounds of Formula (I), (II), a subembodiment as set forth herein, or a pharmaceutically acceptable salt thereof for use in the manufacture of a medicament for the treatment or prevention of a disease in a patient, wherein the disease is characterized by overexpression of Polθ.

In yet another aspect of the inventive concept, provided are compounds of Formula (I), (II), a subembodiment as set forth herein, or a pharmaceutically acceptable salt thereof for use in the manufacture of a medicament for treating a homologous recombinant (HR) deficient cancer in a patient.

In yet another aspect of the inventive concept, provided are compounds of Formula (I), (II), a subembodiment as set forth herein, or a pharmaceutically acceptable salt thereof for use in the manufacture of a medicament for treatment or prevention of a cancer in a patient, wherein the cancer is characterized by a reduction or absence of BRCA gene expression, the absence of the BRCA gene, or reduced function of BRCA protein.

In yet another aspect of the inventive concept, provided are compounds of Formula (I), (II), a subembodiment as set forth herein, or a pharmaceutically acceptable salt thereof for use in the manufacture of a medicament for the treatment or prevention of a cancer that is resistant to poly(ADP-ribose)polymerase (PARP) inhibitor therapy in a patient.

In yet another aspect of the inventive concept, provided herein are methods of identifying Polθ polymerase domain inhibitory activity in a test compound, said method including:
(i) contacting the test compound and Polθ polymerase domain (residues 1819-2590) in an assay buffer to form a reaction pre-mixture;
(ii) contacting the reaction pre-mixture of (i) with (a) a dNTP substrate mixture, and (b) a primed molecular beacon DNA to form a test solution, wherein the primed molecular beacon DNA comprises a labeled template annealed to a primer, wherein the labeled template is SEQ ID NO:1 (5′-CCTTCCTCCCGTGTCTTGTACCTTCCCGTCAGGAGGAAGG-3′) having one or more fluorescent labels, and the primer is SEQ ID NO:3 (5′-GACGGGAAGG-3′); and
(iii) measuring fluorescence intensity of the test reaction mixture, wherein said method further comprises performing steps (i)-(iii) with a positive control sample represented by Formula (I), (II), or any subembodiments as set forth herein.

Other aspects, features, and advantages of the present inventive concept will be apparent to a person of skill in the art upon review of the following detailed description.

### DETAILED DESCRIPTION

Before the present inventive concept is further described, it is to be understood that the inventive concept is not limited to the particular embodiments set forth herein, and it is also to be understood that the terminology used herein is for the purpose of describing particular and/or exemplary embodiments only, and is not intended to be limiting.

Where a range of values is provided, it is understood that each intervening value, to the tenth of the unit of the lower limit unless the context clearly dictates otherwise, between the upper and lower limit of that range and any other stated or intervening value in that stated range, is encompassed within the inventive concept. The upper and lower limits of these smaller ranges may independently be included in the smaller ranges, and are also encompassed within the inventive concept, subject to any specifically excluded limit in the stated range. Where the stated range includes one or both of the limits, ranges excluding either or both of those included limits are also included in the inventive concept. Unless defined otherwise, all technical and scientific terms used herein have the same meaning as commonly understood by one of ordinary skill in the art to which this inventive concept belongs.

The publications discussed herein are provided solely for their disclosure prior to the filing date of the present application. Further, the dates of publication provided may be different from the actual publication dates, which may need to be independently confirmed.

### Definitions

Unless otherwise stated, the following terms used in the specification and claims are defined for the purposes of this Application and have the following meaning:

The singular forms “a,” “an,” and “the” as used herein and in the appended claims include plural referents unless the context clearly dictates otherwise. The term “and/or” includes any and all combinations of one, or more, of the associated listed items and may be abbreviated as “/”. It is further noted that the claims may be drafted to exclude any optional element. As such, this statement is intended to serve as antecedent basis for use of such exclusive terminology such as “solely,” “only” and the like in connection with the recitation of claim elements, or use of a “negative” limitation.

The term “comprise,” as used herein, in addition to its regular meaning, may also include, and, in some embodiments, may specifically refer to the expressions “consist essentially of” and/or “consist of.” Thus, the expression “comprise” can also refer to embodiments, wherein that which is claimed “comprises” specifically listed elements does not include further elements, as well as embodiments wherein that which is claimed “comprises” specifically listed elements may and/or does encompass further elements, or encompass further elements that do not materially affect the basic and novel characteristic(s) of that which is claimed. For example, that which is claimed, such as a method, kit, system, etc. “comprising” specifically listed elements also encompasses, for example, a method, kit, system, etc. “consisting of,” i.e., wherein that which is claimed does not include further elements, and, for example, a method, kit, system, etc. “consisting essentially of,” i.e., wherein that which is claimed may include further elements that do not materially affect the basic and novel characteristic(s) of that which is claimed.

“Alkyl” means a linear saturated monovalent hydrocarbon radical of one to eight carbon atoms or a branched saturated monovalent hydrocarbon radical of three to six carbon atoms, e.g., methyl, ethyl, propyl, 2-propyl, butyl, pentyl, and the like. It will be recognized by a person skilled in the art that the term “alkyl” may include “alkylene” groups.

“Alkylene” means a linear saturated divalent hydrocarbon radical of one to six carbon atoms or a branched saturated divalent hydrocarbon radical of three to six carbon atoms unless otherwise stated e.g., methylene, ethylene, propylene, 1-methylpropylene, 2-methylpropylene, butylene, pentylene, and the like.

“Alkoxy” means a —OR radical where R is alkyl as defined above, e.g., methoxy, ethoxy, propoxy, or 2-propoxy, n-, iso-, or tert-butoxy, and the like.

“Cycloalkyl” means a monocyclic monovalent hydrocarbon radical of three to six carbon atoms which may be saturated or contain one double bond. Cycloalkyl may be unsubstituted or substituted with one or two substituents independently selected from alkyl, halo, alkoxy, hydroxy, or cyano. Examples include, but are not limited to, cyclopropyl, cyclobutyl, cyclopentyl, cyclohexyl, 1-cyanocycloprop-1-yl, 1-cyanomethylcycloprop-1-yl, 3-fluorocyclohexyl, and the like. When cycloalkyl contains a double bond, it may be referred to herein as cycloalkenyl.

“Halo” means fluoro, chloro, bromo, or iodo, preferably fluoro or chloro.

“Haloalkyl” means alkyl radical as defined above, which is substituted with one to five halogen atoms, such as fluorine or chlorine, including those substituted with different halogens, e.g., —CH₂Cl, —CF₃, —CHF₂, —CH₂CF₃, —CF₂CF₃, —CF(CH₃)₂, and the like. When the alkyl is substituted with only fluoro, it can be referred to in this Application as fluoroalkyl.

“Haloalkoxy” means a —OR radical where R is haloalkyl as defined above e.g., —OCF₃, —OCHF₂, and the like. When R is haloalkyl where the alkyl is substituted with only fluoro, it is referred to in this Application as fluoroalkoxy.

“Heterocycloalkyl” means a monocyclic or bicyclic ring system having from 3 ring members to 10 ring members and from 1 to about 5 heteroatom ring vertices selected from N, O and S. The heteroatoms can also be oxidized, such as, but not limited to, —S(O)— and —S(O)₂—. Heterocycloalkyl moieties can be saturated or include one double bond. For example, heterocycloalkyl groups include, but are not limited to, tetrahydrofuranyl, tetrahydrothiophenyl, morpholino, pyrrolidinyl, pyrrolinyl, imidazolidinyl, pyrazolidinyl, pyrazolinyl, piperazinyl, and piperidinyl.

“Hydroxyalkyl” means a linear monovalent hydrocarbon radical of one to six carbon atoms or a branched monovalent hydrocarbon radical of three to six carbons substituted with one or two hydroxy groups, provided that if two hydroxy groups are present they are not both on the same carbon atom. Representative examples include, but are not limited to, hydroxymethyl, 2-hydroxy-ethyl, 2-hydroxypropyl, 3-hydroxypropyl, 1-(hydroxymethyl)-2-methylpropyl, 2-hydroxybutyl, 3-hydroxybutyl, 4-hydroxybutyl, 2,3-dihydroxypropyl, 1-(hydroxymethyl)-2-hydroxyethyl, 2,3-dihydroxybutyl, 3,4-dihydroxybutyl and 2-(hydroxymethyl)-3-hydroxypropyl, preferably 2-hydroxyethyl, 2,3-dihydroxypropyl, and 1-(hydroxymethyl)-2-hydroxyethyl.

“Heteroaryl” means a monovalent monocyclic or bicyclic aromatic radical of 5 to 10 ring atoms, unless otherwise stated, where one or more, (in one embodiment, one, two, or three), ring atoms are heteroatom selected from N, O, or S, the remaining ring atoms being carbon, unless stated otherwise. Non-limiting examples of heteroaryl groups include pyridyl, pyridazinyl, pyrazinyl, pyrimindinyl, triazinyl, quinolinyl, quinoxalinyl, quinazolinyl, cinnolinyl, phthalazinyl, benzotriazinyl, purinyl, benzimidazolyl, benzopyrazolyl, benzotriazolyl, benzisoxazolyl, isobenzofuryl, isoindolyl, indolizinyl, benzotriazinyl, thienopyridinyl, thienopyrimidinyl, pyrazolopyrimidinyl, imidazopyridines, benzothiaxolyl, benzofuranyl, benzothienyl, indolyl, quinolyl, isoquinolyl, isothiazolyl, pyrazolyl, indazolyl, pteridinyl, imidazolyl, triazolyl, tetrazolyl, oxazolyl, isoxazolyl, thiadiazolyl, pyrrolyl, thiazolyl, furyl, thienyl, and the like. As defined herein, the terms “heteroaryl” and “aryl” are mutually exclusive. When the heteroaryl ring contains 5- or 6 ring atoms it is also referred to herein as 5-or 6-membered heteroaryl.

“Heterocyclyl” means a saturated or unsaturated monovalent monocyclic group of 4 to 8 ring atoms in which one or two ring atoms are heteroatom selected from N, O, or S(O)ₙ, where n is an integer from 0 to 2, the remaining ring atoms being C. Additionally, one or two ring carbon atoms in the heterocyclyl ring can optionally be replaced by a —CO— group. More specifically the term heterocyclyl includes, but is not limited to, azetidinyl, oxetanyl, pyrrolidino, piperidino, homopiperidino, 2-oxopyrrolidinyl, 2-oxopiperidinyl, morpholino, piperazino, tetrahydro-pyranyl, thiomorpholino, and the like. When the heterocyclyl ring is unsaturated it can contain one or two ring double bonds provided that the ring is not aromatic.

“Oxo,” as used herein, alone or in combination, refers to =(0).

When needed, any definition herein may be used in combination with any other definition to describe a composite structural group. By convention, the trailing element of any such definition is that which attaches to the parent moiety. For example, the composite group alkoxyalkyl means that an alkoxy group is attached to the parent molecule through an alkyl group.

“Pharmaceutically acceptable salts” refers to salts that retain the desired biological activity of the subject compound and exhibit minimal undesired toxicological effects. Pharmaceutically acceptable salts include salts of the active compounds which are prepared with relatively nontoxic acids or bases, depending on the particular substituents found on the compounds as set forth herein. When compounds as set forth herein contain relatively acidic functionalities, base addition salts can be obtained by contacting the neutral form of such compounds with a sufficient amount of the desired base, either neat or in a suitable inert solvent. Examples of salts derived from pharmaceutically acceptable inorganic bases include aluminum, ammonium, calcium, copper, ferric, ferrous, lithium, magnesium, manganic, manganous, potassium, sodium, zinc and the like. Salts derived from pharmaceutically-acceptable organic bases include salts of primary, secondary and tertiary amines, including substituted amines, cyclic amines, naturally-occurring amines and the like, such as arginine, betaine, caffeine, choline, N,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like. When compounds of the present inventive concept contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogen carbonic, phosphoric, monohydrogen phosphoric, dihydrogen phosphoric, sulfuric, monohydrogen sulfuric, hydriodic, or phosphorous acids and the like, as well as the salts derived from relatively nontoxic organic acids like acetic, propionic, isobutyric, malonic, benzoic, succinic, suberic, fumaric, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge, S. M., et al, “Pharmaceutical Salts”, Journal of Pharmaceutical Science, 1977, 66, 1-19; P L Gould, International Journal of Pharmaceutics, 1986, 33, 201-217; and Bighley et al, Encyclopaedia of Pharmaceutical Technology, Marcel Dekker Inc, New York 1996, Volume 13, page 453-497). Other salts that are not deemed pharmaceutically acceptable may be useful in the preparation of compounds of Formula (I), (II), and any embodiment thereof as set forth herein, including specific compounds, and are included within the scope of the inventive concept, such as ammonia and trifluoroacetic acid. The present inventive concept encompasses all possible stoichiometric and non-stoichiometric forms of the salts of the compounds of Formula (I), (II), and any embodiment thereof as set forth herein. Certain specific compounds of the present inventive concept contain both basic and acidic functionalities that allow the compounds to be converted into either base or acid addition salts.

The neutral forms of the compounds may be regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present inventive concept.

The present disclosure also includes protected derivatives of compounds of the present disclosure. For example, when compounds of the present disclosure contain groups such as hydroxy, carboxy, thiol or any group containing a nitrogen atom(s), these groups can be protected with a suitable protecting group. A comprehensive list of suitable protective groups can be found in T. W. Greene, Protective Groups in Organic Synthesis, 5^{th} Ed., John Wiley & Sons, Inc. (2014), the disclosure of which is incorporated herein by reference in its entirety. The protected derivatives of compounds of the present disclosure can be prepared by methods well known in the art.

The present disclosure also includes prodrugs of the compound of Formula (I), (II), and any embodiment thereof as set forth herein including specific compounds, or a pharmaceutically acceptable salt thereof. Prodrugs of the compounds as set forth herein are those compounds that readily undergo chemical changes under physiological conditions to provide the compounds of the present inventive concept. An example, without limitation, of a prodrug would be a compound which is administered as an ester (the “prodrug”), but then is metabolically hydrolyzed to the carboxylic acid, the active entity. Additionally, prodrugs can be converted to the compounds of the present inventive concept by chemical or biochemical methods in an ex vivo environment. For example, prodrugs can be slowly converted to the compounds of the present inventive concept when placed in a transdermal patch reservoir with a suitable enzyme or chemical reagent. Pharmaceutically acceptable prodrugs are described in T. Higuchi and V. Stella, Prodrugs as Novel Delivery Systems, Vol. 14 of the A.C.S. Symposium Series, Edward B. Roche, ed., Bioreversible Carriers in Drug Design, American Pharmaceutical Association and Pergamon Press, 1987, and in D. Fleisher, S. Ramon and H. Barbra “Improved oral drug delivery: solubility limitations overcome by the use of prodrugs”, Advanced Drug Delivery Reviews, 1996, 19(2), 115-130, each of which are incorporated herein by reference.

Prodrugs may be any covalently bonded carriers that release a compound of of Formula (I), (II), any embodiment thereof as set forth herein including specific compounds, or pharmaceutically acceptable salt thereof in vivo when such prodrug is administered to a patient. Prodrugs are generally prepared by modifying functional groups in a way such that the modification is cleaved, either by routine manipulation or in vivo, yielding the parent compound. Prodrugs include, for example, compounds of this inventive concept wherein hydroxy, amine or sulfhydryl groups are bonded to any group that, when administered to a patient, cleaves to form the hydroxy, amine or sulfhydryl groups. Thus, representative examples of prodrugs include (but are not limited to) acetate, formate and benzoate derivatives of alcohol, sulfhydryl and amine functional groups of the compounds of formula (I). Further, in the case of a carboxylic acid (—COOH), esters may be employed, such as methyl esters, ethyl esters, and the like. Esters may be active in their own right and/or be hydrolysable under in vivo conditions in the human body. Suitable pharmaceutically acceptable in vivo hydrolysable ester groups include those which break down readily in the human body to leave the parent acid or its salt.

Certain compounds of Formula (I), (II), and any embodiment thereof as set forth herein can form complexes with solvents in which they are reacted or from which they are precipitated or crystallized. These complexes are known as “solvates”. Where the solvent is water, the complex is known as a “hydrate.” In general, the solvated forms are equivalent to unsolvated forms and are intended to be encompassed within the scope of the present inventive concept. Certain compounds of Formula (I), (II), any embodiment thereof as set forth herein including specific compounds, or pharmaceutically acceptable salt thereof may exist in multiple crystalline or amorphous forms. In general, all physical forms are equivalent for the uses contemplated by the present disclosure and are intended to be within the scope of the present disclosure.

Certain compounds of Formula (I), (II) (and any embodiment thereof as set forth herein including specific compounds) possess asymmetric carbon atoms/centers (optical or chiral centers) or double bonds; the racemates, diastereomers, geometric isomers, regioisomers and individual isomers (e.g., separate enantiomers) are all intended to be encompassed within the scope of the present inventive concept. Chiral centers, such as chiral carbon atoms, may also be present in a substituent such as an alkyl group. When a stereochemical depiction is shown, it is meant to refer the compound in which one of the isomers is present and substantially free of the other isomer. ‘Substantially free of’ another isomer indicates at least an 80/20 ratio of the two isomers, more preferably 90/10, or 95/5 or more. In some embodiments, one of the isomers will be present in an amount of at least 99%. Where the stereochemistry of a chiral center present in a compound of Formula (I), (II), any embodiment thereof as set forth herein, or in any chemical structure illustrated herein, is not specified the structure is intended to encompass all individual stereoisomers and all mixtures thereof. Thus, compounds of Formula (I), (II), any embodiment thereof set forth herein, and pharmaceutically acceptable salts thereof containing one or more chiral center may be used as racemic mixtures, enantiomerically enriched mixtures, or as enantiomerically pure individual stereoisomers.

The compounds of Formula (I), (II) (and any embodiment thereof as set forth herein, including specific compounds) may also contain unnatural and/or enriched amounts of isotopes at one or more of the atoms that constitute such compounds. Unnatural and/or enriched amounts of an isotope may be defined as ranging from the amount found in nature to an amount 100% of the atom in question. Exemplary isotopes that can be incorporated into compounds of the present inventive concept, such as a compound of Formula (I), (II), (and any embodiment thereof as set forth herein including specific compounds) include isotopes of hydrogen, carbon, nitrogen, oxygen, phosphorus, sulfur, fluorine, chlorine, and iodine, such as ²H, ³H, ¹¹C, ¹³C, 14C, 13N, ¹⁵N, ¹⁵O, ¹⁷O, ¹⁸O, ³²P, ³³P, ³⁵S, ¹⁸F, ³⁶Cl, ¹²³I and ¹²⁵1, respectively. Isotopically labeled compounds (e.g., those labeled with ³H and ¹⁴C) can be useful in compound or substrate tissue distribution assays. Tritiated (i.e., ³H) and carbon-14 (i.e., ¹⁴C) isotopes can be useful for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements). In some embodiments, in compounds as set forth herein, including in Table 1 below one or more hydrogen atoms are replaced by ²H or ³H, or one or more carbon atoms are replaced by ¹³C- or ¹⁴C-enriched carbon. Positron emitting isotopes such as ¹⁵O, ¹³N, ¹¹C, and ¹⁵F are useful for positron emission tomography (PET) studies to examine substrate receptor occupancy. Isotopically labeled compounds can generally be prepared by following procedures analogous to those as set forth in the Schemes or in the Examples herein, by substituting an isotopically labeled reagent for a non-isotopically labeled reagent.

“Pharmaceutically acceptable carrier or excipient” means a carrier or an excipient that is useful in preparing a pharmaceutical composition that is generally safe, non-toxic and neither biologically nor otherwise undesirable, and includes a carrier or an excipient that is acceptable for veterinary use as well as human pharmaceutical use. “A pharmaceutically acceptable carrier/excipient” as used in the specification and claims includes both one and more than one such excipient.

“About,” as used herein, is intended to qualify the numerical values which it modifies, denoting such a value as variable within a margin of error. When no particular margin of error, such as a standard deviation to a mean value given in a chart or table of data, is recited, the term “about” should be understood to mean that range which may encompass, for example, ±20%, +15%, +10%, and in some embodiments, preferably ±5%, the recited value and the range is included.

“Disease” as used herein is intended to be generally synonymous, and is used interchangeably with, the terms “disorder,” “syndrome,” and “condition” (as in medical condition), in that all reflect an abnormal condition of the human or animal body or of one of its parts that impairs normal functioning, is typically manifested by distinguishing signs and symptoms, and causes the human or animal to have a reduced duration or quality of life.

“Patient” is generally synonymous with the term “subject” or “individual” and as used herein includes all mammals including humans. Examples of patients include humans, livestock such as cows, goats, sheep, pigs, and rabbits, and companion animals such as dogs, cats, rabbits, and horses. Preferably, the patient is a human. Human patients may be any gender or gender identity.

“In need of treatment” as used herein means the patient is being treated by a physician or other caregiver after diagnoses of the disease. For example, the patient has been diagnosed as having a disease linked to overexpression of Polθ or a homologous recombination (HR)-deficient cancer.

“Administration”, “administer” and the like, as they apply to, for example, a patient, cell, tissue, organ, or biological fluid, refer to contact of, for example, a compound of Formula (I), (II), a pharmaceutical composition including the same, or a diagnostic agent to the subject, cell, tissue, organ, or biological fluid. In the context of a cell, administration includes contact (e.g., in vitro or ex vivo) of a reagent to the cell, as well as contact of a reagent to a fluid, where the fluid is in contact with the cell.

“Therapeutically effective amount” as used herein means the amount of a compound of Formula (I), (II) (and any embodiment thereof as set forth herein including specific compounds) or a pharmaceutically acceptable salt thereof that, when administered to a patient for treating a disease either alone or as part of a pharmaceutical composition and either in a single dose or as part of a series of doses, is sufficient to affect such treatment for the disease. The “therapeutically effective amount” will vary depending on the compound, the disease and its severity and the age, weight, etc., of the mammal to be treated. The therapeutically effective amount can be ascertained by measuring relevant physiological effects, and it can be adjusted in connection with the dosing regimen and diagnostic analysis of the subject's condition, and the like. By way of example, measurement of the serum level of a compound of Formula (I), (II), (or, e.g., a metabolite thereof) at a particular time post-administration may be indicative of whether a therapeutically effective amount has been used.

“Treating” or “treatment” of a disease includes:
(1) inhibiting the disease, i.e., arresting or reducing the development of the disease or its clinical symptoms; or
(2) relieving the disease, i.e., causing regression of the disease or its clinical symptoms.

“Inhibiting”, “reducing,” or any variation of these terms in relation of Polθ, includes any measurable decrease or complete inhibition to achieve a desired result. For example, there may be a decrease of about, at most about, or at least about 5%, 10%, 15%, 20%, 25%, 30%, 35%, 40%, 45%, 50%, 55%, 60%, 65%, 70%, 75%, 80%, 85%, 90%, 95%, 99%, or more, or any range derivable therein, reduction of Polθ activity compared to its normal activity.

The term “preventing” refers to causing the clinical symptoms of the disease not to develop in a mammal that may be exposed to or predisposed to the disease but does not yet experience or display symptoms of the disease.

The term “homologous recombination” refers to the cellular process of genetic recombination in which nucleotide sequences are exchanged between two similar or identical DNA.

The term “homologous recombination (HR) deficient cancer” refers to a cancer that is characterized by a reduction or absence of a functional HR repair pathway. HR deficiency may arise from absence of one or more HR-associated genes or presence of one or more mutations in one or more HR-associated genes. Examples of HR-associated genes include BRCA1 BRCA2, RAD54, RAD51B, Ct1P (Choline Transporter-Like Protein), PALB2 (Partner and Localizer of BRCA2), XRCC2 (X-ray repair complementing defective repair in Chinese hamster cells 2), RECQL4 (RecQ Protein-like 4), BLM (Bloom syndrome, RecQ helicase-like), WRN (Werner syndrome, one or more HR-associated genes), Nbs 1 (Nibrin), and genes coding Fanconi anemia (FA) proteins or FA like genes e.g., FANCA, FANCB, FANCC, FANCD1 (BRCA2), FANCD2, FANCE, FANCF, FANCG, FANCI, FANJ (BRIP1), FANCL, FANCM, FANCN (RALB2), FANCP (SLX4), FANCS (BRCA1), RAD51C and XPF.

The term “Polθ overexpression” refers to the increased expression or activity of Polθ enzyme in a diseased cell e.g., cancer cell, relative to expression or activity of Polθ enzyme in a control cell (e.g., non-diseased cell of the same type). The amount of The amount of Polθ overexpression can be at least 2-fold, at least 3-fold, at least 4-fold, at least 5-fold, Polθ overexpression can be at least 2-fold, at least 3-fold, at least 4-fold, at least 5-fold, at least 6-fold, at least 10-fold, at least 20-fold, at least 50-fold, relative to Polθ expression in a control cell. Examples of Polθ overexpressing cancers include, but are not limited to, certain ovarian, breast, cervical, lung, colorectal, gastric, bladder, and prostate cancers.

Compounds of Formula (I) and Formula (II)

In some aspects of the inventive concept, provided herein are compounds of Formula (I) and Formula (II)

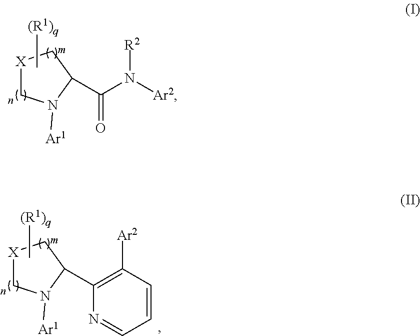

or a pharmaceutically acceptable salt thereof, wherein
X is selected from the group consisting of —CH₂—, —CHR¹—, —NR¹—, —NH—, and —O—;
m is an integer selected from the group consisting of 0, 1, and 2;
n is an integer selected from the group consisting of 0, 1, and 2;
provided that the sum of m and n is at least 1 and no more than 3;
q is an integer selected from the group consisting of 0, 1, and 2;
each R¹ is independently selected from the group consisting of C₁₋₈ alkyl, C₁₋₈ haloalkyl, —OR^{a},
   —X¹—OR^{a}, —NR^{a}R^{b}, —X¹—NR^{a}R^{b}—NR^{a}C(O)R^{b}, —X¹—NR^{a}C(O)R^{b}, —C(O)NR^{a}R^{b}, —X¹—C(O)NR^{a}R^{b}, —C(O)R^{a}, —X¹—C(O)R^{a}, phenyl, and —X¹-phenyl, wherein
   X¹ is C₁₋₃ alkylene;
   each R^{a} and R^{b} are independently selected from the group consisting of H, C₁₋₄ alkyl, and C₁₋₄ haloalkyl; and
   phenyl is substituted with from 0 to 3 R^{c} moieties, each R^{c} is selected from the group consisting of C₁₋₈ alkyl, halo, C₁₋₈ haloalkyl, C₁₋₈ alkoxy, C₁₋₈ haloalkoxy, —OH,
      —X^{c}—OH and cyano, wherein X^{c} is C₁₋₃ alkylene;
Ar¹ is selected from the group consisting of phenyl and 6- to 10-membered heteroaryl having 1 to 4 heteroatom ring vertices independently selected from the group consisting of N, O, and S, wherein
   Ar¹ is substituted with 0 to 4 R^{d} moieties, wherein each R^{d} is independently selected from the group consisting of C₁₋₈ alkyl, halo, C₁₋₈ haloalkyl, cyano, —OR^{e}, —NR^{e}R^{f},
      —NR^{e}C(O)R^{f}, and —C(O)NR^{e}R^{f}, wherein
      each R^{e} and R^{f} are independently selected from the group consisting of H, C₁₋₆ alkyl, and C₁₋₆ haloalkyl;
R², when present, is selected from the group consisting of C₁₋₈ alkyl, C₁₋₈ haloalkyl, C₃₋₆ cycloalkyl, phenyl, and 3- to 6-membered heterocycloalkyl having 1 to 3 heteroatom ring vertices independently selected from the group consisting of N, O, and S; and
Ar² is selected from the group consisting of phenyl and 6- to 10-membered heteroaryl having 1 to 4 heteroatom ring vertices independently selected from the group consisting of N, O, and S, wherein
   Ar² is substituted with 0 to 4 R^{h} moieties, wherein each R^{h} is independently selected from the group consisting of C₁₋₈ alkyl, C₁₋₈ haloalkyl, halo, cyano, C₃₋₆ cycloalkyl,
      —ORⁱ, —NR^{j}R^{k}, —NR^{j}C(O)R^{k}, and —C(O)NR^{j}R^{k}, wherein
   each Rⁱ is selected from the group consisting of H, C₁₋₆ alkyl, C₁₋₆ haloalkyl, and C₃₋₆ cycloalkyl; and
   each R^{j} and R^{k} are independently selected from the group consisting of H, C₁₋₆ alkyl, and C₁₋₆ haloalkyl.

In some embodiments, the compounds as set forth herein are represented by Formula (I)

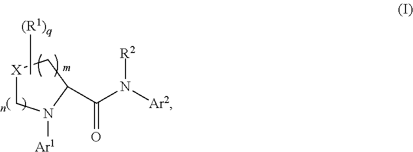

or a pharmaceutically acceptable salt thereof.

In some embodiments, the compounds as set forth herein are represented by Formula (II)

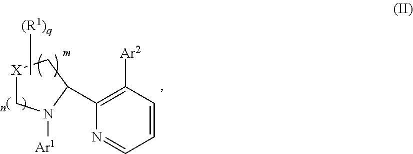

or a pharmaceutically acceptable salt thereof.

In some embodiments, the compounds as set forth herein are represented by Formula (Ia)

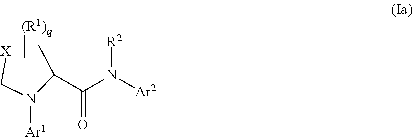

or a pharmaceutically acceptable salt thereof. In some embodiments, if the compounds as set forth herein are represented by Formula (Ia), then Ar¹ is not 2-pyridyl or 2-pyrimidinyl, or Ar² is not phenyl or 2-pyridyl.

In some embodiments, if Ar¹ is 2-pyridyl or 2-pyrimidinyl in the compounds as set forth herein represented by Formula (Ia), then Ar¹ is substituted with 1 to 4 R^{d} moieties, wherein each R^{d} is independently —NR^{e}C(O)R^{f}, or —C(O)NR^{e}R^{f}. In some embodiments, if Ar² is phenyl or 2-pyridyl in the compounds as set forth herein represented by Formula (Ia), then Ar² is substituted with 1 to 4 R^{h} moieties wherein each R^{h} is independently —NR^{j}C(O)R^{k}, or —C(O)NR^{j}R^{k}.

In some embodiments, the compounds as set forth herein are represented by Formula (Ia-1)

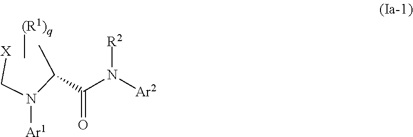

or a pharmaceutically acceptable salt thereof.

In some embodiments, the compounds as set forth herein are represented by Formula (Ia-2)

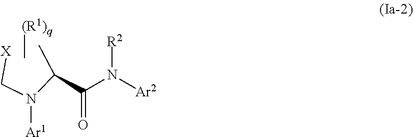

or a pharmaceutically acceptable salt thereof. In some embodiments, if the compounds as set forth herein are represented by Formula (Ia-2), then Ar¹ is not 2-pyridyl or 2-pyrimidinyl, or Ar² is not phenyl or 2-pyridyl.

In some embodiments, if Ar¹ is 2-pyridyl or 2-pyrimidinyl in the compounds as set forth herein represented by Formula (Ia-2), then Ar¹ is substituted with 1 to 4 R^{d} moieties, wherein each R^{d} is indepndently —NR^{e}C(O)R^{f}, or —C(O)NR^{e}R^{f}. In some embodiments, if Ar² is phenyl or 2-pyridyl in the compounds as set forth herein represented by Formula (Ia-2), then Ar² is substituted with 1 to 4 R^{h} moieties wherein each R^{h} is independently —NR^{j}C(O)R^{k}, or

—C(O)NR^{j}R^{k}.

In some embodiments, the compounds as set forth herein are represented by Formula (Ib)

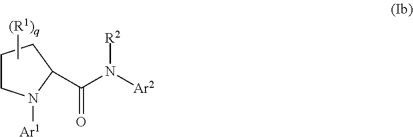

or a pharmaceutically acceptable salt thereof. In some embodiments, if the compounds as set forth herein are represented by Formula (Ib), then Ar¹ is not 2-pyridyl or 2-pyrimidinyl, or Ar² is not phenyl or 2-pyridyl.

In some embodiments, if Ar¹ is 2-pyridyl or 2-pyrimidinyl in the compounds as set forth herein represented by Formula (Ib), then Ar¹ is substituted with 1 to 4 R^{d} moieties, wherein each R^{d} is independently —NR^{e}C(O)R^{f}, or —C(O)NR^{e}R^{f}. In some embodiments, if Ar² is phenyl or 2-pyridyl in the compounds as set forth herein represented by Formula (Ib), then Ar² is substituted with 1 to 4 R^{h} moieties wherein each R^{h} is independently —NR^{j}C(O)R^{k}, or —C(O)NR^{j}R^{k}.

In some embodiments, the compounds as set forth herein are represented by Formula (Ib-1)

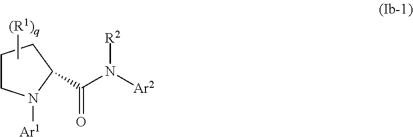

or a pharmaceutically acceptable salt thereof.

In some embodiments, the compounds as set forth herein are represented by Formula (Ib-2)

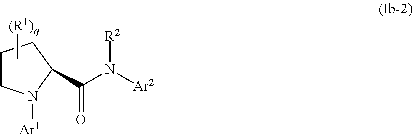

or a pharmaceutically acceptable salt thereof. In some embodiments, if the compounds as set forth herein are represented by Formula (Ib-2), then Ar¹ is not 2-pyridyl or 2-pyrimidinyl, or Ar² is not phenyl or 2-pyridyl.

In some embodiments, if Ar¹ is 2-pyridyl or 2-pyrimidinyl in the compounds as set forth herein represented by Formula (Ib-2), then Ar¹ is substituted with 1 to 4 R^{d} moieties, wherein each R^{d} is independently —NR^{e}C(O)R^{f}, or —C(O)NR^{e}R^{f}. In some embodiments, if Ar² is phenyl or 2-pyridyl in the compounds as set forth herein represented by Formula (Ib-2), then Ar² is substituted with 1 to 4 R^{h} moieties wherein each R^{h} is independently —NR^{j}C(O)R^{k}, or —C(O)NRⱼR^{k}.

In some embodiments, the compounds as set forth herein are represented by Formula (Ib-i)

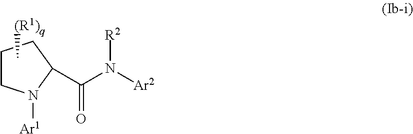

or a pharmaceutically acceptable salt thereof. In some embodiments, if the compounds as set forth herein are represented by Formula (Ib-i), then Ar¹ is not 2-pyridyl or 2-pyrimidinyl, or Ar² is not phenyl or 2-pyridyl.

In some embodiments, if Ar¹ is 2-pyridyl or 2-pyrimidinyl in the compounds as set forth herein represented by Formula (Ib-i), then Ar¹ is substituted with 1 to 4 R^{d} moieties, wherein each R^{d} is independently —NR^{e}C(O)R^{f}, or —C(O)NR^{e}R^{f}. In some embodiments, if Ar² is phenyl or 2-pyridyl in the compounds as set forth herein represented by Formula (Ib-i), then Ar² is substituted with 1 to 4 R^{h} moieties wherein each R^{h} is independently —NR^{j}C(O)R^{k}, or

—C(O)NR^{j}R^{k}.

In some embodiments, the compounds as set forth herein are represented by Formula (Ib-ii)

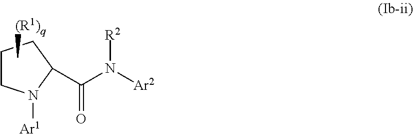

or a pharmaceutically acceptable salt thereof. In some embodiments, if the compounds as set forth herein are represented by Formula (Ib-ii), then Ar¹ is not 2-pyridyl or 2-pyrimidinyl, or Ar² is not phenyl or 2-pyridyl.

In some embodiments, if Ar¹ is 2-pyridyl or 2-pyrimidinyl in the compounds as set forth herein represented by Formula (Ib-ii), then Ar¹ is substituted with 1 to 4 R^{d} moieties, wherein each R^{d} is independently —NR^{e}C(O)R^{f}, or —C(O)NR^{e}R^{f}. In some embodiments, if Ar² is phenyl or 2-pyridyl in the compounds as set forth herein represented by Formula (Ib-ii), then Ar² is substituted with 1 to 4 R^{h} moieties wherein each R^{h} is independently —NR^{j}C(O)R^{k}, or —C(O)NR^{j}R^{k}.

In some embodiments, the compounds as set forth herein are represented by Formula (Ib-l1)

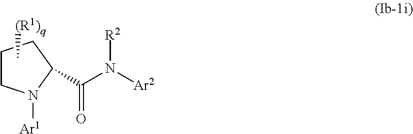

or a pharmaceutically acceptable salt thereof.

In some embodiments, the compounds as set forth herein are represented by Formula (Ib-1ii)

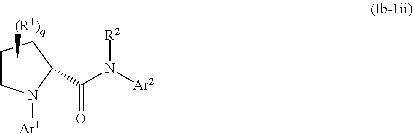

or a pharmaceutically acceptable salt thereof.

In some embodiments, the compounds as set forth herein are represented by Formula (Ib-2i)

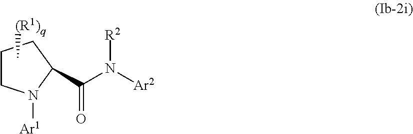

or a pharmaceutically acceptable salt thereof. In some embodiments, if the compounds as set forth herein are represented by Formula (Ib-2i), then Ar¹ is not 2-pyridyl or 2-pyrimidinyl, or Ar² is not phenyl or 2-pyridyl.

In some embodiments, if Ar¹ is 2-pyridyl or 2-pyrimidinyl in the compounds as set forth herein represented by Formula (Ib-2i), then Ar¹ is substituted with 1 to 4 R^{d} moieties, wherein each R^{d} is independently —NR^{e}C(O)R^{f}, or —C(O)NR^{e}R^{f}. In some embodiments, if Ar² is phenyl or 2-pyridyl in the compounds as set forth herein represented by Formula (Ib-21), then Ar² is substituted with 1 to 4 R^{h} moieties wherein each R^{h} is independently —NR^{j}C(O)R^{k}, or —C(O)NR^{j}R^{k}.

In some embodiments, the compounds as set forth herein are represented by Formula (Ib-2ii)

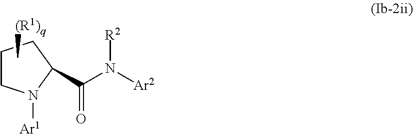

or a pharmaceutically acceptable salt thereof. In some embodiments, if the compounds as set forth herein are represented by Formula (Ib-2ii), then Ar¹ is not 2-pyridyl or 2-pyrimidinyl, or Ar² is not phenyl or 2-pyridyl.

In some embodiments, if Ar¹ is 2-pyridyl or 2-pyrimidinyl in the compounds as set forth herein represented by Formula (Ib-2ii), then Ar¹ is substituted with 1 to 4 R^{d} moieties, wherein each R^{d} is independently —NR^{e}C(O)R^{f}, or —C(O)NR^{e}R^{f}. In some embodiments, if Ar² is phenyl or 2-pyridyl in the compounds as set forth herein represented by Formula (Ib-2ii), then Ar² is substituted with 1 to 4 R^{h} moieties wherein each R^{h} is independently —NR^{j}C(O)R^{k}, or —C(O)NR^{j}R^{k}.

In some embodiments, the compounds as set forth herein are represented by Formula (Ic)

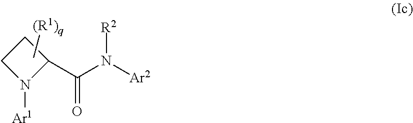

or a pharmaceutically acceptable salt thereof.

In some embodiments, the compounds as set forth herein are represented by Formula (Ic-1)

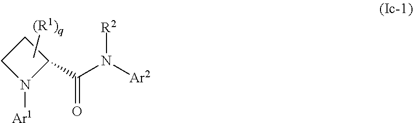

or a pharmaceutically acceptable salt thereof.

In some embodiments, the compounds as set forth herein are represented by Formula (Ic-2)

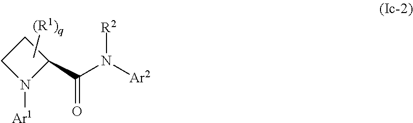

or a pharmaceutically acceptable salt thereof.

In some embodiments, the compounds as set forth herein are represented by Formula (Ic-i)

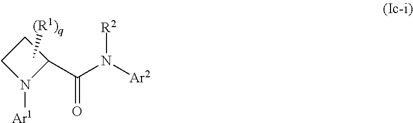

or a pharmaceutically acceptable salt thereof.

In some embodiments, the compounds as set forth herein are represented by Formula (Ic-ii)

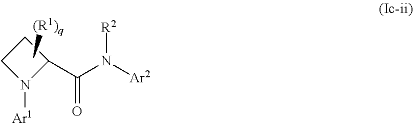

or a pharmaceutically acceptable salt thereof.

In some embodiments, the compounds as set forth herein are represented by Formula (Ic-l1)

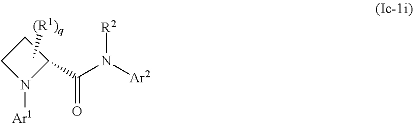

or a pharmaceutically acceptable salt thereof.

In some embodiments, the compounds as set forth herein are represented by Formula (Ic-1ii)

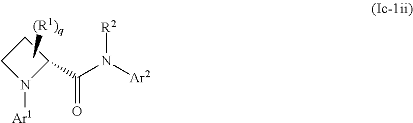

or a pharmaceutically acceptable salt thereof.

In some embodiments, the compounds as set forth herein are represented by Formula (Ic-2i)

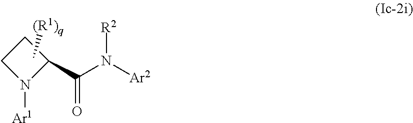

or a pharmaceutically acceptable salt thereof.

In some embodiments, the compounds as set forth herein are represented by Formula (Ic-2ii)

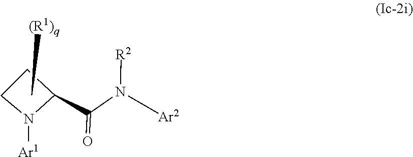

or a pharmaceutically acceptable salt thereof.

In some embodiments, the compounds as set forth herein are represented by Formula (Id)

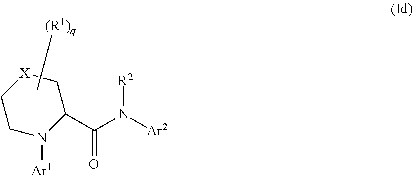

or a pharmaceutically acceptable salt thereof.

In some embodiments, the compounds as set forth herein are represented by Formula (Id-1)

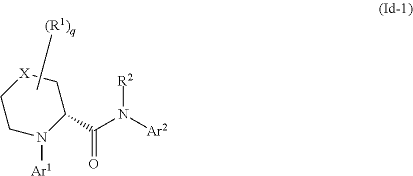

or a pharmaceutically acceptable salt thereof.

In some embodiments, the compounds as set forth herein are represented by Formula (Id-2)

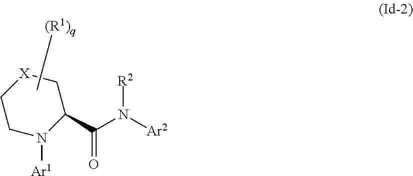

or a pharmaceutically acceptable salt thereof.

In some embodiments, the compounds as set forth herein are represented by Formula (Id-i)

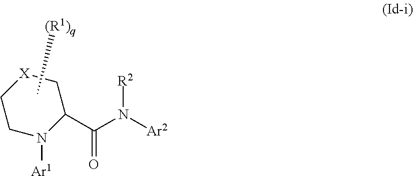

or a pharmaceutically acceptable salt thereof.

In some embodiments, the compounds as set forth herein are represented by Formula (Id-ii)

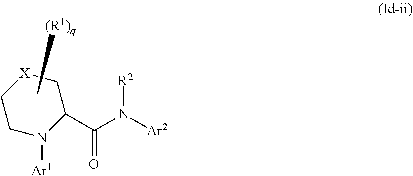

or a pharmaceutically acceptable salt thereof.

In some embodiments, the compounds as set forth herein are represented by Formula (Id-l1)

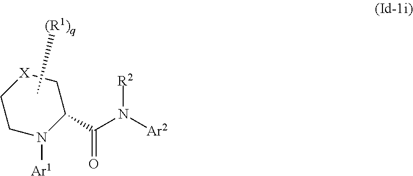

or a pharmaceutically acceptable salt thereof.

In some embodiments, the compounds as set forth herein are represented by Formula (Id-1ii)

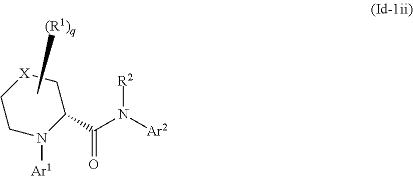

or a pharmaceutically acceptable salt thereof.

In some embodiments, the compounds as set forth herein are represented by Formula (Id-2i)

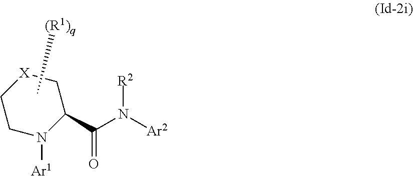

or a pharmaceutically acceptable salt thereof.

In some embodiments, the compounds as set forth herein are represented by Formula (Id-2ii)

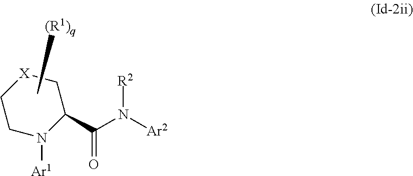

or a pharmaceutically acceptable salt thereof.

In some embodiments, the compounds as set forth herein are represented by Formula (Ie)

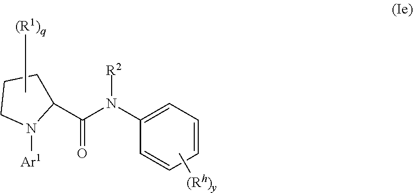

or a pharmaceutically acceptable salt thereof, wherein
y is 0, 1, or 2.

In some embodiments, if the compounds as set forth herein are represented by Formula (Ie), then Ar¹ is not 2-pyridyl or 2-pyrimidinyl.

In some embodiments, if Ar¹ is 2-pyridyl or 2-pyrimidinyl in the compounds as set forth herein represented by Formula (Ie), then Ar¹ is substituted with 1 to 4 R^{d} moieties, wherein each R^{d} is independently —NR^{e}C(O)R^{f}, or —C(O)NR^{e}R^{f}. In some embodiments of the compounds as set forth herein represented by Formula (Ie), y is 1 or 2 and R^{h} is independently —NR^{j}C(O)R^{k}, or —C(O)NR^{j}R^{k}.

In some embodiments, the compounds as set forth herein are represented by Formula (If)

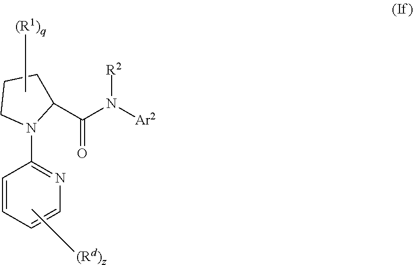

or a pharmaceutically acceptable salt thereof, wherein
z is 0, 1, or 2.

In some embodiments, if the compounds as set forth herein are represented by Formula (If), then Ar² is not phenyl or 2-pyridyl.

In some embodiments, if Ar² is phenyl or 2-pyridyl in the compounds as set forth herein represented by Formula (If), then Ar² is substituted with 1 to 4 R^{h} moieties wherein each R^{h} is —NR^{j}C(O)R^{k}, or —C(O)NR^{j}R^{k}. In some embodiments of the compounds as set forth herein represented by Formula (If), z is 1 or 2 and R^{d} is independently —NR^{e}C(O)R^{f}, or —C(O)NR^{e}R^{f}.

In some embodiments, the compounds as set forth herein are represented by Formula (Ig)

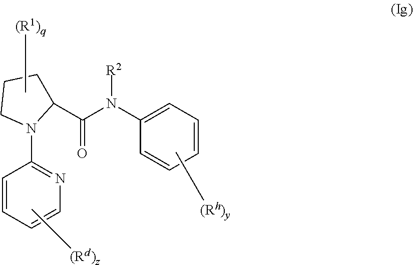

or a pharmaceutically acceptable salt thereof, wherein
y is 0, 1, or 2; and
z is 0, 1, or 2.

In some embodiments, if the compounds as set forth herein are represented by Formula (Ig), then z is 1 or 2 and each R^{d} is independently —NR^{e}C(O)R^{f} or —C(O)NR^{e}R^{f}, or y is 1 or 2 and each R^{h} is independently —NR^{j}C(O)R^{k} or —C(O)NR^{j}R^{k}.

In some embodiments, the compounds as set forth herein are represented by Formula (IIa):

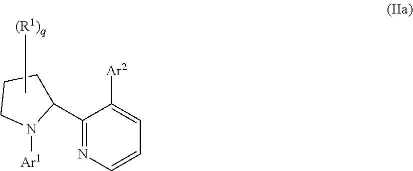

or a pharmaceutically acceptable salt thereof.

In some embodiments, the compounds as set forth herein are represented by Formula (IIb):

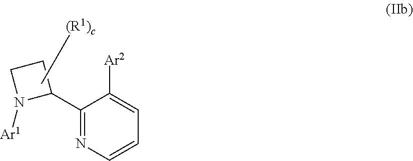

or a pharmaceutically acceptable salt thereof.

In some embodiments, the compounds as set forth herein are represented by Formula (IIc)

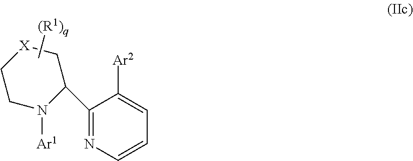

or a pharmaceutically acceptable salt thereof.

In some embodiments of Formula (I) and (II) and relevant subembodiments thereof, X is —CH₂—. In some embodiments of Formula (I) and (II) and relevant subembodiments thereof, X is —NH—. In some embodiments of Formula (I) and (II) and relevant subembodiments thereof, X is —O—.

In some embodiments of Formula (I) and (II) and relevant subembodiments thereof, q is 0 or 1. In some embodiments of Formula (I) and (II) and relevant subembodiments thereof, q is 1. In some embodiments of Formula (I) and (II) and relevant subembodiments thereof, q is 0.

In some embodiments of Formula (I) and (II) and relevant subembodiments thereof, each Rⁱ is independently selected from the group consisting of C₁₋₈ alkyl, C₁₋₈ haloalkyl, —OR^{a}, —X¹—OR^{a}, —NR^{a}R^{b}, —X¹—NR^{a}R^{b}, —NR^{a}C(O)R^{b}, —X¹—NR^{a}C(O)R^{b}, —C(O)N^{a}R^{b}, —X¹—C(O)NR^{a}R^{b}, —C(O)R^{a}, and —X¹—C(O)R^{a}, wherein
X¹ is C₁₋₃ alkylene; and
each R^{a} and R^{b} are independently selected from the group consisting of H, C₁₋₄ alkyl, and C₁₋₄ haloalkyl.

In some embodiments of Formula (I) and (II) and relevant subembodiments thereof, each R¹ is independently selected from the group consisting of C₁₋₈ alkyl, C₁₋₈ haloalkyl, —OR^{a}, —NR^{a}R^{b}—NR^{a}C(O)R^{b}, —C(O)NR^{a}R^{b}, and —C(O)R^{a}, wherein
each R^{a} and R^{b} are independently selected from the group consisting of H, C₁₋₄ alkyl, and C₁₋₄ haloalkyl.

In some embodiments of Formula (I) and relevant subembodiments thereof, when X is selected from the group consisting of —CH₂—, —NH—, and —O—; then
each R¹ is independently selected from the group consisting of C₁₋₈ haloalkyl, —NR^{a}R^{b}, —X¹—NR^{a}R^{b}, —NR^{a}C(O)R^{b}, —X¹—NR^{a}C(O)R^{b}, —C(O)NR^{a}R^{b}, —X¹—C(O)NR^{a}R^{b}, —C(O)R^{a}, —X¹—C(O)R^{a}, phenyl, and —X¹-phenyl, wherein
   X¹ is C₁₋₃ alkylene;
   each R^{a} and R^{b} are independently selected from the group consisting of H, C₁₋₄ alkyl, and C₁₋₄ haloalkyl; and
   phenyl is substituted with from 0 to 3 R^{c} moieties, each R^{c} is selected from the group consisting of C₁₋₈ alkyl, halo, C₁₋₈ haloalkyl, C₁₋₈ alkoxy, C₁₋₈ haloalkoxy, —OH,
      —X^{c}—OH, and cyano, wherein X^{c} is C₁₋₃ alkylene.

In some embodiments of Formula (I) and relevant subembodiments thereof, R², when present, is selected from the group consisting of C₁₋₈ alkyl, C₁₋₈ haloalkyl, C₃₋₆ cycloalkyl, phenyl, and 3- to 6-membered heterocycloalkyl having 1 to 3 heteroatom ring vertices independently selected from the group consisting of N, O, and S.

In some embodiments of Formula (I) and relevant subembodiments thereof, R², when present, is selected from the group consisting of C₁₋₈ alkyl, and C₁₋₈ haloalkyl.

In some embodiments of Formula (I) and relevant subembodiments thereof, R², when present, is selected from the group consisting of C₃₋₆ cycloalkyl, phenyl, and 3- to 6-membered heterocycloalkyl having 1 to 3 heteroatom ring vertices independently selected from the group consisting of N, O, and S.

In some embodiments of Formula (I) and relevant subembodiments thereof, R², when present, is selected from the group consisting of phenyl, and 3- to 6-membered heterocycloalkyl having 1 to 3 heteroatom ring vertices independently selected from the group consisting of N, O, and S.

In some embodiments of Formula (I) and (II) and relevant subembodiments thereof, Ar¹ is phenyl substituted with 0 to 3 R^{d} moieties.

In some embodiments of Formula (I) and (II) and relevant subembodiments thereof, Ar¹ is a 6- to 10-membered heteroaryl having 1 to 4 heteroatom ring vertices independently selected from the group consisting of N, O, and S, the 6- to 10-membered heteroaryl is substituted with 0 to 3 R^{d} moieties.

In some embodiments of Formula (I) and (II) and relevant subembodiments thereof, Ar¹ a 6-membered heteroaryl having 1 to 4 heteroatom ring vertices independently selected from the group consisting of N, O, and S, the 6-membered heteroaryl is substituted with 0 to 3 R^{d} moieties.

In some embodiments of Formula (I) and (II) and relevant subembodiments thereof, Ar¹ is pyridine or pyrimidine substituted with 0 to 3 R^{d} moieties.

In some embodiments of Formula (I) and (II) and relevant subembodiments thereof, Ar¹ is

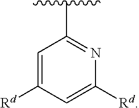

In some embodiments of Formula (I) and (II) and relevant subembodiments thereof, each R^{d}, when present, is independently selected from the group consisting of C₁₋₈ alkyl, halo, C₁₋₈ haloalkyl, cyano, —OR^{e}, and —NR^{e}R^{f}.

In some embodiments of Formula (I) and (II) and relevant subembodiments thereof, each R^{d}, when present, is independently selected from the group consisting of C₁₋₈ alkyl, halo, C₁₋₈ haloalkyl, and cyano.

In some embodiments of Formula (I) and relevant subembodiments thereof, R² is selected from the group consisting of C₁₋₄ alkyl, C₁₋₄ haloalkyl, C₃₋₆ cycloalkyl, phenyl, and 3- to 6-membered heterocycloalkyl having 1 to 3 heteroatom ring vertices independently selected from the group consisting of N, O, and S.

In some embodiments of Formula (I) and relevant subembodiments thereof, R² is selected from the group consisting of C₁₋₄ alkyl, C₁₋₄ haloalkyl, C₃₋₆ cycloalkyl, and 3- to 6-membered heterocycloalkyl having 1 to 3 heteroatom ring vertices independently selected from the group consisting of N, O, and S.

In some embodiments of Formula (I) and relevant subembodiments thereof, R² is selected from the group consisting of C₁₋₂ alkyl, C₁₋₂ haloalkyl, cycloproyl, and oxetanyl.

In some embodiments of Formula (I) and (II) and relevant subembodiments thereof, Ar² is phenyl substituted with 0 to 3 R^{h} moieties.

In some embodiments of Formula (I) and (II) and relevant subembodiments thereof, Ar² is selected from the group consisting of

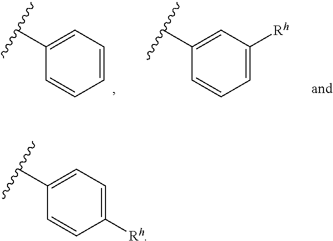

In some embodiments of Formula (I) and (II) and relevant subembodiments thereof, Ar² is 6- to 10-membered heteroaryl having 1 to 4 heteroatom ring vertices independently selected from the group consisting of N, O, and S, the 6- to 10-membered heteroaryl is substituted with 0 to 3 R^{h} moieties.

In some embodiments of Formula (I) and (II) and relevant subembodiments thereof, Ar² is a 6-membered heteroaryl having 1 to 3 heteroatom ring vertices independently selected from the group consisting of N, O, and S, the 6-membered heteroaryl is substituted with 0 to 3 R^{h} moieties.

In some embodiments of Formula (I) and (II) and relevant subembodiments thereof, Ar² is selected from the group consisting of pyridine, and benzofuranyl substituted with 0 to 3 R^{h} moieties.

In some embodiments of Formula (I) and (II) and relevant subembodiments thereof, each R^{h}, when present, is independently selected from the group consisting of C₁₋₈ alkyl, C₁₋₈ haloalkyl, halo, cyano, and C₃₋₆ cycloalkyl.

In some embodiments of Formula (I) and (II) and relevant subembodiments thereof, each R^{h}, when present, is independently selected from the group consisting of C₁₋₄ alkyl, C₁₋₄ haloalkyl, halo, and cyano.

In some embodiments of Formula (I) and relevant subembodiments thereof, each R^{h}, when present, is independently selected from the group consisting of cyano, —ORⁱ, —NR^{j}R^{k}, —NR^{j}C(O)R^{k}, and —C(O)NR^{j}R^{k}, wherein each Rⁱ is selected from the group consisting of C₁₋₆ haloalkyl, and C₃₋₆ cycloalkyl; and each R^{j} and R^{k} are independently selected from the group consisting of H, C₁₋₆ alkyl, and C₁₋₆ haloalkyl.

In some embodiments of Formula (I) and relevant subembodiments thereof, each R^{h}, when present, is independently selected from the group consisting of —ORⁱ, —NR^{j}R^{k}, —NR^{j}C(O)R^{k}, and —C(O)NR^{j}R^{k}, wherein each R is selected from the group consisting of C₁₋₆ haloalkyl, and C₃₋₆ cycloalkyl; and each R^{j} and R^{k} are independently selected from the group consisting of H, C₁₋₆ alkyl, and C₁₋₆ haloalkyl.

In some embodiments of Formula (I) and (II) and relevant subembodiments thereof, each R^{h}, when present, is cyano.

In some embodiments of Formula (I) and (II) and relevant subembodiments thereof, each R^{h}, when present, is halo.

In some embodiments of Formula (I) and (II) and relevant subembodiments thereof, each R^{h}, when present, is F.

In some embodiments of Formula (I) and (II) and relevant subembodiments thereof, each R^{h}, when present, is Br.

Representative compound of Formula (I) and Formula (II) are listed in Table 1 below:

TABLE 1

| Cpd. | | |
|---|---|---|
| No. | Structure | Name |
| 1.001 | 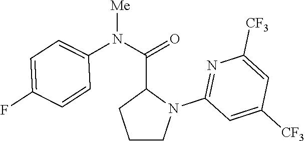 [SMILES: CN(C(=O)C1CCCN1c1cc(C(F)(F)F)cc(C(F)(F)F)n1)c1ccc(F)cc1] | 1-(4,6-bis(trifluoromethyl)pyridin- 2-yl)-N-(4-fluorophenyl)-N-methyl- pyrrolidine-2-carboxamide |
| 1.002 | 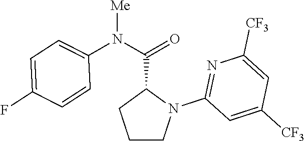 [SMILES: CN(C(=O)[C@H]1CCCN1c1cc(C(F)(F)F)cc(C(F)(F)F)n1)c1ccc(F)cc1] | (R)-1-(4,6-bis(trifluoromethyl)- pyridin-2-yl)-N-(4-fluorophenyl)-N- methylpyrrolidine-2-carboxamide |
| 1.003 | 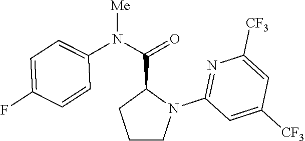 [SMILES: CN(C(=O)[C@@H]1CCCN1c1cc(C(F)(F)F)cc(C(F)(F)F)n1)c1ccc(F)cc1] | (S)-1-(4,6-bis(trifluoromethyl)- pyridin-2-yl)-N-(4-fluorophenyl)-N- methylpyrrolidine-2-carboxamide |
| 1.004 | 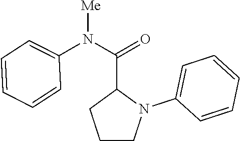 [SMILES: CN(C(=O)C1CCCN1c1ccccc1)c1ccccc1] | N-methyl-N,1-diphenylpyrrolidine- 2-carboxamide |
| 1.005 | 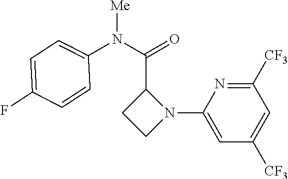 [SMILES: CN(C(=O)C1CCN1c1cc(C(F)(F)F)cc(C(F)(F)F)n1)c1ccc(F)cc1] | 1-(4,6-bis(trifluoromethyl)pyridin- 2-yl)-N-(4-fluorophenyl)-N-methyl- azetidine-2-carboxamide |
| 1.006 | 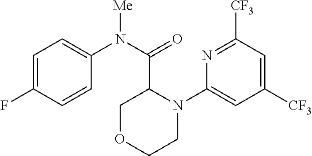 [SMILES: CN(C(=O)C1COCCN1c1cc(C(F)(F)F)cc(C(F)(F)F)n1)c1ccc(F)cc1] | 4-(4,6-bis(trifluoromethyl)pyridin- 2-yl)-N-(4-fluorophenyl)-N-methyl- morpholine-3-carboxamide |
| 1.007 | 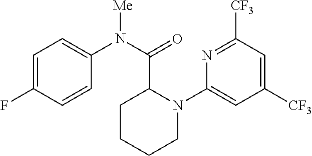 [SMILES: CN(C(=O)C1CCCCN1c1cc(C(F)(F)F)cc(C(F)(F)F)n1)c1ccc(F)cc1] | 1-(4,6-bis(trifluoromethyl)pyridin- 2-yl)-N-(4-fluorophenyl)-N-methyl- piperidine-2-carboxamide |
| 1.008 | 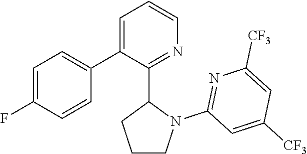 [SMILES: Fc1ccc(-c2cccnc2C2CCCN2c2cc(C(F)(F)F)cc(C(F)(F)F)n2)cc1] | 2-(2-(3-(4-fluorophenyl)pyridin-2- yl)pyrrolidin-1-yl)-4,6-bis(trifluoro- methyl)pyridine |
| 1.009 | 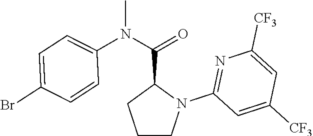 [SMILES: CN(C(=O)[C@@H]1CCCN1c1cc(C(F)(F)F)cc(C(F)(F)F)n1)c1ccc(Br)cc1] | (S)-1-(4,6-bis(trifluoromethyl)- pyridin-2-yl)-N-(4-bromophenyl)-N- methylpyrrolidine-2-carboxamide |
| 1.010 | 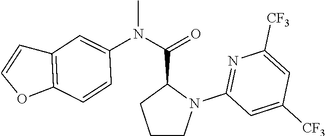 [SMILES: CN(C(=O)[C@@H]1CCCN1c1cc(C(F)(F)F)cc(C(F)(F)F)n1)c1ccc2occc2c1] | (S)-N-(benzofuran-5-yl)-1-(4,6- bis(trifluoromethyl)pyridin-2-yl)-N- methylpyrrolidine-2-carboxamide |
| 1.011 | 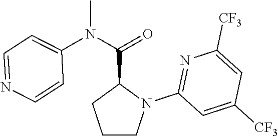 [SMILES: CN(C(=O)[C@@H]1CCCN1c1cc(C(F)(F)F)cc(C(F)(F)F)n1)c1ccncc1] | (S)-1-(4,6-bis(trifluoromethyl)- pyridin-2-yl)-N-methyl-N-(pyridin-4- yl)pyrrolidine-2-carboxamide |
| 1.012 | 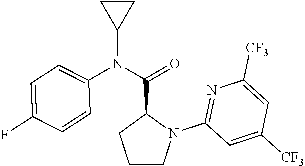 [SMILES: O=C([C@@H]1CCCN1c1cc(C(F)(F)F)cc(C(F)(F)F)n1)N(c1ccc(F)cc1)C1CC1] | (S)-1-(4,6-bis(trifluoromethyl)- pyridin-2-yl)-N-cyclopropyl-N-(4- fluorophenyl)pyrrolidine-2-carbox- amide |
| 1.013 | 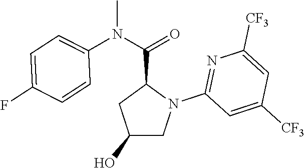 [SMILES: CN(C(=O)[C@@H]1C[C@H](O)CN1c1cc(C(F)(F)F)cc(C(F)(F)F)n1)c1ccc(F)cc1] | (2S,4S)-1-(4,6-bis(trifluoromethyl)- pyridin-2-yl)-N-(4-fluorophenyl)-4- hydroxy-N-methylpyrrolidine-2- carboxamide |
| 1.014 | 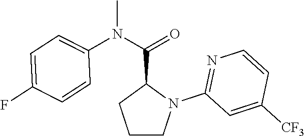 [SMILES: CN(C(=O)[C@@H]1CCCN1c1cc(C(F)(F)F)ccn1)c1ccc(F)cc1] | (S)-N-(4-fluorophenyl)-N-methyl-1- (4-(trifluoromethyl)pyridin-2-yl)- pyrrolidine-2-carboxamide |
| 1.015 | 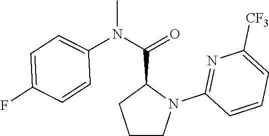 [SMILES: CN(C(=O)[C@@H]1CCCN1c1cccc(C(F)(F)F)n1)c1ccc(F)cc1] | (S)-N-(4-fluorophenyl)-N-methyl-1- (6-(trifluoromethyl)pyridin-2-yl)- pyrrolidine-2-carboxamide |
| 1.016 | 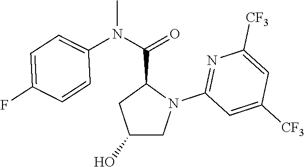 [SMILES: CN(C(=O)[C@@H]1C[C@@H](O)CN1c1cc(C(F)(F)F)cc(C(F)(F)F)n1)c1ccc(F)cc1] | (2S,4R)-1-(4,6-bis(trifluoromethyl)- pyridin-2-yl)-N-(4-fluorophenyl)-4- hydroxy-N-methylpyrrolidine-2- carboxamide |
| 1.017 | 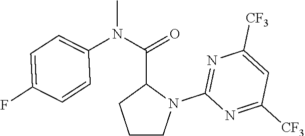 [SMILES: CN(C(=O)C1CCCN1c1nc(C(F)(F)F)cc(C(F)(F)F)n1)c1ccc(F)cc1] | 1-(4,6-bis(trifluoromethyl)pyrimidin- 2-yl)-N-(4-fluorophenyl)-N-methyl- pyrrolidine-2-carboxamide |
| 1.018 | 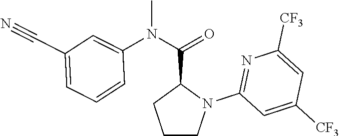 [SMILES: CN(C(=O)[C@@H]1CCCN1c1cc(C(F)(F)F)cc(C(F)(F)F)n1)c1cccc(C#N)c1] | (S)-1-(4,6-bis(trifluoromethyl)- pyridin-2-yl)-N-(3-cyanophenyl)-N- methylpyrrolidine-2-carboxamide |
| 1.019 | 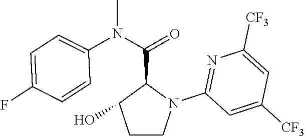 [SMILES: CN(C(=O)[C@@H]1[C@@H](O)CCN1c1cc(C(F)(F)F)cc(C(F)(F)F)n1)c1ccc(F)cc1] | (2S,3S)-1-(4,6-bis(trifluoromethyl)- pyridin-2-yl)-N-(4-fluorophenyl)-3- hydroxy-N-methylpyrrolidine-2- carboxamide |
| 1.020 | 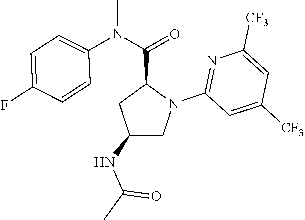 [SMILES: CC(=O)N[C@H]1C[C@@H](C(=O)N(C)c2ccc(F)cc2)N(c2cc(C(F)(F)F)cc(C(F)(F)F)n2)C1] | (2S,4S)-4-acetamido-1-(4,6- bis(trifluoromethyl)pyridin-2-yl)- N-(4-fluorophenyl)-N-methyl- pyrrolidine-2-carboxamide |
| 1.021 | 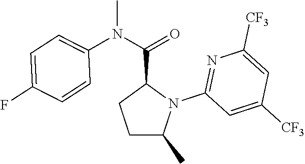 [SMILES: C[C@H]1CC[C@@H](C(=O)N(C)c2ccc(F)cc2)N1c1cc(C(F)(F)F)cc(C(F)(F)F)n1] | (2S,5S)-1-(4,6-bis(trifluoromethyl)- pyridin-2-yl)-N-(4-fluorophenyl)- N,5-dimethylpyrrolidine-2-carbox- amide |
| 1.022 | 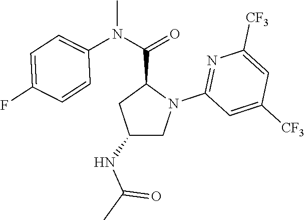 [SMILES: CC(=O)N[C@@H]1C[C@@H](C(=O)N(C)c2ccc(F)cc2)N(c2cc(C(F)(F)F)cc(C(F)(F)F)n2)C1] | (2S,4R)-4-acetamido-1-(4,6- bis(trifluoromethyl)pyridin-2-yl)- N-(4-fluorophenyl)-N-methyl- pyrrolidine-2-carboxamide |
| 1.023 | 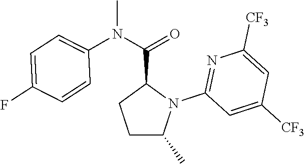 [SMILES: C[C@@H]1CC[C@@H](C(=O)N(C)c2ccc(F)cc2)N1c1cc(C(F)(F)F)cc(C(F)(F)F)n1] | (2S,5R)-1-(4,6-bis(trifluoromethyl)- pyridin-2-yl)-N-(4-fluorophenyl)- N,5-dimethylpyrrolidine-2-carbox- amide |
| 1.024 | 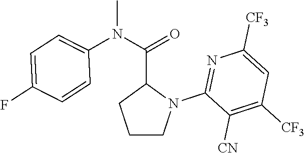 [SMILES: CN(C(=O)C1CCCN1c1nc(C(F)(F)F)cc(C(F)(F)F)c1C#N)c1ccc(F)cc1] | 1-(3-cyano-4,6-bis(trifluoromethyl)- pyridin-2-yl)-N-(4-fluorophenyl)-N- methylpyrrolidine-2-carboxamide |
| 1.025 | 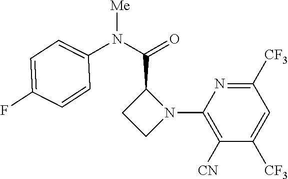 [SMILES: CN(C(=O)[C@@H]1CCN1c1nc(C(F)(F)F)cc(C(F)(F)F)c1C#N)c1ccc(F)cc1] | (S)-1-(3-cyano-4,6-bis(trifluoro- methyl)-pyridin-2-yl)-N-(4-fluoro- phenyl)-N-methylazetidine-2- carboxamide |
| 1.026 | 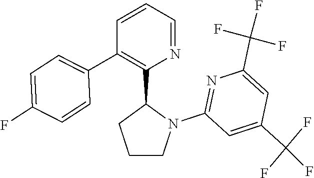 [SMILES: Fc1ccc(-c2cccnc2[C@@H]2CCCN2c2cc(C(F)(F)F)cc(C(F)(F)F)n2)cc1] | (S)-2-(2-(3-(4-fluorophenyl)- pyridin-2-yl)pyrrolidin-1-yl)-4,6- bis(trifluoromethyl)pyridine |
| 1.027 | 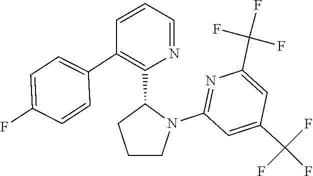 [SMILES: Fc1ccc(-c2cccnc2[C@H]2CCCN2c2cc(C(F)(F)F)cc(C(F)(F)F)n2)cc1] | (R)-2-(2-(3-(4-fluorophenyl)- pyridin-2-yl)pyrrolidin-1-yl)-4,6- bis(trifluoromethyl)pyridine |
| 1.028 | 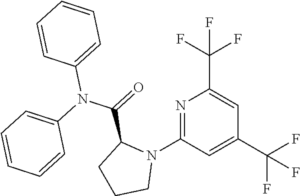 [SMILES: O=C([C@@H]1CCCN1c1cc(C(F)(F)F)cc(C(F)(F)F)n1)N(c1ccccc1)c1ccccc1] | (S)-1-(4,6-bis(trifluoromethyl)- pyridin-2-yl)-N,N-diphenylpyrrol- idine-2-carboxamide |
| 1.029 | 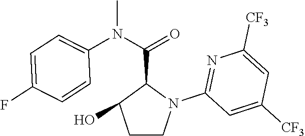 [SMILES: CN(C(=O)[C@@H]1[C@H](O)CCN1c1cc(C(F)(F)F)cc(C(F)(F)F)n1)c1ccc(F)cc1] | (2S,3R)-1-(4,6-bis(trifluoromethyl)- pyridin-2-yl)-N-(4-fluorophenyl)-3- hydroxy-N-methylpyrrolidine-2- carboxamide |
| 1.030 | 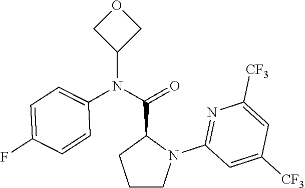 [SMILES: O=C([C@@H]1CCCN1c1cc(C(F)(F)F)cc(C(F)(F)F)n1)N(c1ccc(F)cc1)C1COC1] | (S)-1-(4,6-bis(trifluoromethyl)- pyridin-2-yl)-N-(4-fluorophenyl)-N- (oxetan-3-yl)pyrrolidine-2-carbox- amide |
| 1.031 | 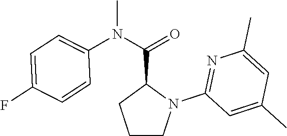 [SMILES: Cc1cc(C)nc(N2CCC[C@H]2C(=O)N(C)c2ccc(F)cc2)c1] | (S)-1-(4,6-dimethylpyridin-2-yl)-N- (4-fluorophenyl)-N-methylpyrrol- idine-2-carboxamide |

General Synthetic Schemes

Compounds of this disclosure can be made by the methods depicted in the reaction schemes shown below.

The starting materials and reagents used in preparing these compounds are either available from commercial suppliers such as Aldrich Chemical Co., (Milwaukee, Wis.), Bachem (Torrance, Calif), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), March's Advanced Organic Chemistry, (John Wiley and Sons, 4th Edition) and Larock's Comprehensive Organic Transformations (VCH Publishers Inc., 1989). These schemes are merely illustrative of some methods by which the compounds of this disclosure can be synthesized, and various modifications to these schemes can be made and will be suggested to one skilled in the art reading this disclosure. The starting materials and the intermediates, and the final products of the reaction may be isolated and purified if desired using conventional techniques, including but not limited to filtration, distillation, crystallization, chromatography and the like. Such materials may be characterized using conventional means, including physical constants and spectral data.

Unless specified to the contrary, the reactions as set forth herein take place at atmospheric pressure over a temperature range from about −78° C. to about 150° C., such as from about 0° C. to about 125° C. and further such as at about room (or ambient) temperature, e.g., about 20° C.

Compounds of Formula (I) can be prepared by the method illustrated as set forth in Scheme 1 below.

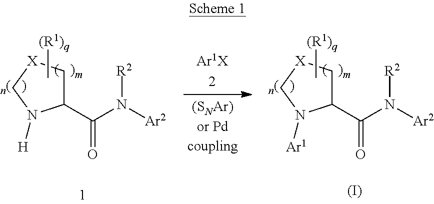

Compounds of Formula (I) can be prepared by reacting a cyclic amide of formula 1 or it's salt with an arylhalide of formula 2 where Ar¹ is as defined in the Summary in the presence of a base such as N-methylpyridine, diethylisopropylamine, pyridine, and the like, or under Palladium reaction conditions well known in the art. Compounds of formula 1 can be prepared by reacting an amine of formula Ar²R²NH where Ar² is as defined in the Summary with an cyclic carboxylic acid of formula A

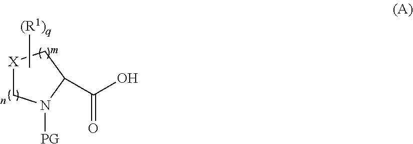

where PG is a nitrogen protecting group such as Boc, Cbz and the like and R¹ is as defined in the Summary under amino acid coupling reaction conditions, followed by removal of the amino protecting group to provide a compound of formula 1.

Compounds of Formula (II) can be prepared by the method illustrated as set forth in Scheme 2 below.

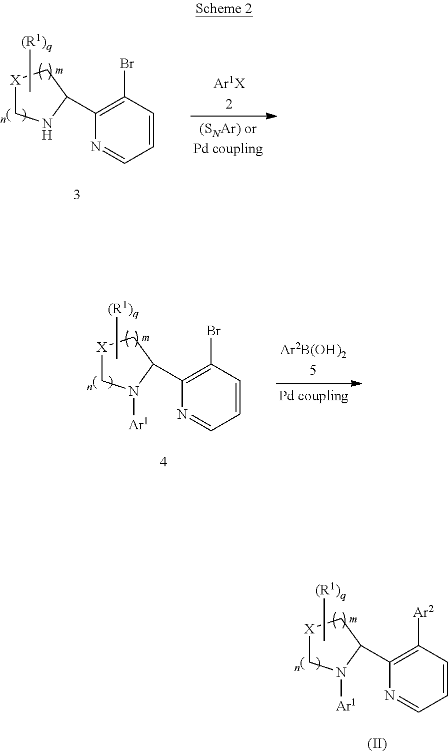

Compounds of Formula 4 can be prepared by reacting a cyclic amine of formula 3 or it's salt with an arylhalide of formula 2 where Ar¹ is as defined in the Summary in the presence of a base such as N-methylpyridine, diethylisopropylamine, pyridine, and the like, or under Palladium reaction conditions well known in the art. Compounds of Formula 4 can be treated with aryl boronic acids of Formula 5 where Ar² is as defined in the Summary under Palladium coupling conditions, well known in the art, to form compounds of Formula (I). Compounds of Formula 3 are commercially available or can be prepared by methods well known in the art.

Methods of Use

In some embodiments of the inventive concept, provided are methods for treating and/or preventing a disease characterized by overexpression of Polθ in a patient including administering to the patient a therapeutically effective amount of a compound of Formula (I), (II), a subembodiment described herein, or a pharmaceutically acceptable thereof; or a pharmaceutical composition including a compound of Formula (I) or a compound of Formula (II) and at least one pharmaceutically acceptable excipient.

In further embodiments the inventive concept, the patient is in recognized need of such treatment. In some embodiments, the compound of Formula (I), (II), a subembodiment described herein, or a pharmaceutically acceptable salt thereof is administered in a pharmaceutical composition. In some embodiments, the disease is a cancer.

In still further embodiments of the inventive concept, provided are methods of treating and/or preventing a homologous recombinant (HR) deficient cancer in a patient including administering to the patient a therapeutically effective amount of a compound of Formula (I), (II), a subembodiment described herein, or a pharmaceutically acceptable salt thereof. In first embodiment of the fourth aspect, the patient is in recognized need of such treatment. In second embodiment of the fourth aspect and first embodiment contained therein, the compound of Formula (I), (II), a subembodiment described herein, or a pharmaceutically acceptable salt thereof is administered in a pharmaceutical composition.

In still further embodiments of the inventive concept, provided are methods for inhibiting DNA repair by Polθ in a cancer cell including contacting the cell with an effective amount of a compound of Formula (I), (II), a subembodiment described herein, or a pharmaceutically acceptable salt thereof. In a first embodiment, the cancer is HR deficient cancer.

In still further embodiments of the inventive concept, provided are methods for treating and/or preventing a cancer in a patient, wherein the cancer is characterized by a reduction or absence of BRCA gene expression, the absence of the BRCA gene, or reduced function of BRCA protein, including administering to the subject a therapeutically effective amount of a compound of Formula (I), (II), a subembodiment described herein, or a pharmaceutically acceptable salt thereof optionally in a pharmaceutical composition.

In still further embodiments of the inventive concept, provided are compounds of Formula (I), (II), a subembodiment described herein, or a pharmaceutically acceptable salt thereof for inhibiting DNA repair by Polθ in a cell. In some embodiments, the cell is HR deficient cell.

In still further embodiments of the inventive concept, provided are compounds of Formula (I), (II), a subembodiment described herein, or a pharmaceutically acceptable salt thereof for use in the treatment and/or prevention of a disease in a patient, wherein the disease is characterized by overexpression of Pol.

In still further embodiments of the inventive concept, provided are compounds of Formula (I), (II), a subembodiment described herein, or a pharmaceutically acceptable salt thereof for use in the treatment and/or prevention of a cancer in a patient, wherein the cancer is characterized by a reduction or absence of BRCA gene expression, the absence of the BRCA gene, or reduced function of BRCA protein.

In still further embodiments of the inventive concept, provided are compounds of Formula (I), (II), a subembodiment described herein, or a pharmaceutically acceptable salt thereof for use in the treatment and/or prevention of a HR deficient cancer in a patient.

In still further embodiments of the inventive concept, provided are compounds of Formula (I), (II), a subembodiment described herein, or a pharmaceutically acceptable salt thereof for use in the treatment or prevention of a cancer that is resistant to poly(ADP-ribose)polymerase (PARP) inhibitor therapy in a patient. Examples of cancers that are resistant to PARP-inhibitors include, but are not limited to, breast cancer, ovarian cancer, lung cancer, bladder cancer, liver cancer, head and neck cancer, pancreatic cancer, gastrointestinal cancer and colorectal cancer.

In still further embodiments of the inventive concept, the cancer is lymphoma, soft tissue, rhabdoid, multiple myeloma, uterus, gastric, peripheral nervous system, rhabdomyosarcoma, bone, colorectal, mesothelioma, breast, ovarian, lung, fibroblast, central nervous system, urinary tract, upper aerodigestive, leukemia, kidney, skin, esophagus, and pancreas (data from large scale drop out screens in cancer cell lines indicate that some cell lines from the above cancers are dependent on polymerase theta for proliferation see https://depmap.org/portal/).

In some embodiments, a HR-deficient cancer is breast cancer. Breast cancer includes, but is not limited to, lobular carcinoma in situ, a ductal carcinoma in situ, an invasive ductal carcinoma, triple negative, HER positive, estrogen receptor positive, progesterone receptor positive, HER and estrogen receptor positive, HER and estrogen and progesterone receptor, positive inflammatory breast cancer, Paget disease of nipple, Phyllodes tumor, angiosarcoma, adenoid cystic carcinoma, low-grade adenosquamous carcinoma, medullary carcinoma, mucinous carcinoma, papillary carcinoma, tubular carcinoma, metaplastic carcinoma, micropapillary carcinoma, and mixed carcinoma. In second embodiment, HR-deficient cancer is ovarian cancer. Ovarian can includes, but is not limited to, epithelial ovarian carcinomas, maturing teratomas, dysgerminomas, endodermal sinus tumors, granulosa-theca tumors, Sertoli-Leydig cell tumors, and primary peritoneal carcinoma.

In some embodiments of the inventive concept, provided are use of the compounds of Formula (I), (II), a subembodiment described herein, or a pharmaceutically acceptable salt thereof in the manufacture of a medicament for use in the treatment or prevention of the methods and uses described herein. This includes, for example, the treatment or prevention of a disease characterized by overexpression of Polθ; a homologous recombinant (HR) deficient cancer; a cancer in a patient, wherein the cancer is characterized by a reduction or absence of BRCA gene expression, the absence of the BRCA gene, or reduced function of BRCA protein; a cancer that is resistant to poly(ADP-ribose)polymerase (PARP) inhibitor therapy in a patient.

Assays

The ability of compounds of the disclosure to inhibit Polθ can be measured as set forth in Biological Example 1 below.

In some embodiments, provided herein are methods of identifying Polθ polymerase domain inhibitory activity in a test compound, said methods including
(i) contacting the test compound and Polθ polymerase domain (residues 1819-2590) in an assay buffer to form a reaction pre-mixture;
(ii) contacting the reaction pre-mixture of (i) with (a) a dNTP substrate mixture, and (b) a primed molecular beacon DNA to form a test solution, wherein the primed molecular beacon DNA comprises a labeled template annealed to a primer, wherein the labeled template is SEQ ID NO:1 (5′-CCTTCCTCCCGTGTCTTGTACCTTCCCGTCAGGAGGAAGG-3′) having one or more fluorescent labels, and the primer is SEQ ID NO:3 (5′-GACGGGAAGG-3′); and
(iii) measuring fluorescence intensity of the test reaction mixture, wherein said method further comprises performing steps (i)-(iii) with a positive control sample represented by Formula (I), Formula (II) (or any embodiments thereof).

In some embodiments, the final concentration of Polθ polymerase domain in the test reaction mixture is 4 nM.

In some embodiments, the assay buffer is 20 m M TRIS, pH 7.80, 50 mM KCl, 10 mM MgCl₂, 1 mM DTT, 0.01% BSA, 0.01% Tween20.

In some embodiments, the dNTP substrate mixture is an equal mixture of each natural dNTP (dTTP, dATP, dCTP, and dGTP). In some embodiments the dNTP in the substrate mixture is 48 μM.

In some embodiments the labeled template is fluorescently labeled with one or more fluorescent labels. A number of fluorescent labels (and quenchers) are known in the art. In some embodiments the one or more fluorescent labels comprise 5′-TAMRA and/or 3′-BHQ. In some embodiments the sequence of the labeled template is SEQ ID NO:2: 5′-CCTTCCTCCCGTGTCTTGTACCTTCCCGTCAGGAGGAAGG-3′ with 5′-TAMRA and 3′-BHQ labels.

In some embodiments the primed molecular beacon DNA further comprises a priming buffer. In some embodiments, the buffer is 10 mM Tris-HCl pH 8.0, 100 mM NaCl buffer, and the concentration of the primed molecular beacon DNA is 96 nM.

A person of skill in the art will recognize that the fluorescence measured will depent on the labels being used in the assay. In some embodiments, absorbance ( [symbol] =485 nm, [symbol] =535 nm) of the Pol theta reaction mixture.

Pharmaceutical Compositions

The compounds of Formula (I), (II), a subembodiment as set forth herein, or a pharmaceutically acceptable salt thereof, may be provided in the form of compositions suitable for administration to a subject. In general, such compositions are pharmaceutical compositions including a compound of Formula (I), (II), a subembodiment as at forth herein, or a pharmaceutically acceptable salt thereof, and one or more pharmaceutically acceptable or physiologically acceptable excipients. In certain embodiments, the compound of Formula (I), (II), a subembodiment described herein, or a pharmaceutically acceptable salt thereof is present in a therapeutically effective amount. The pharmaceutical compositions may be used in the methods as set forth herein; thus, for example, the pharmaceutical compositions can be administered ex vivo or in vivo to a subject in order to practice the therapeutic methods and uses described herein.

The pharmaceutical compositions can be formulated to be compatible with the intended method or route of administration; exemplary routes of administration are set forth herein. Furthermore, the pharmaceutical compositions may be used in combination with other therapeutically active agents or compounds as set forth herein in order to treat the diseases, disorders and conditions contemplated by the present disclosure.

The pharmaceutical compositions containing the active ingredient (e.g., a compound of Formula (I), (II), a subembodiment as set forth herein, a pharmaceutically acceptable salt thereof) may be in a form suitable for oral use, for example, as tablets, capsules, troches, lozenges, aqueous or oily suspensions, dispersible powders or granules, emulsions, hard or soft capsules, or syrups, solutions, microbeads or elixirs. Pharmaceutical compositions intended for oral use may be prepared according to any method known to the art for the manufacture of pharmaceutical compositions, and such compositions may contain one or more agents such as, for example, sweetening agents, flavoring agents, coloring agents and preserving agents in order to provide pharmaceutically elegant and palatable preparations. Tablets, capsules and the like contain the active ingredient in admixture with non-toxic pharmaceutically acceptable excipients which are suitable for the manufacture of tablets, capsules, and the like. These excipients may be, for example, diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc.

The tablets, capsules and the like suitable for oral administration may be uncoated or coated by known techniques to delay disintegration and absorption in the gastrointestinal tract and thereby provide a sustained action. For example, a time-delay material such as glyceryl monostearate or glyceryl di-stearate may be employed. The tablets may also be coated by techniques known in the art to form osmotic therapeutic tablets for controlled release. Additional agents include biodegradable or biocompatible particles or a polymeric substance such as polyesters, polyamine acids, hydrogel, polyvinyl pyrrolidone, polyanhydrides, polyglycolic acid, ethylene-vinyl acetate, methylcellulose, carboxymethylcellulose, protamine sulfate, or lactide and glycolide copolymers, polylactide and glycolide copolymers, or ethylene vinyl acetate copolymers in order to control delivery of an administered composition. For example, the oral agent can be entrapped in microcapsules prepared by coacervation techniques or by interfacial polymerization, by the use of hydroxymethyl cellulose or gelatin-microcapsules or poly (methyl methacrylate) microcapsules, respectively, or in a colloid drug delivery system. Colloidal dispersion systems include macromolecule complexes, nanocapsules, microspheres, microbeads, and lipid-based systems, including oil-in-water emulsions, micelles, mixed micelles, and liposomes. Methods for the preparation of the above-mentioned formulations are known in the art.

Formulations for oral use may also be presented as hard gelatin capsules wherein the active ingredient is mixed with an inert solid diluent, for example, calcium carbonate, calcium phosphate, kaolin or microcrystalline cellulose, or as soft gelatin capsules wherein the active ingredient is mixed with water or an oil medium, for example peanut oil, liquid paraffin, or olive oil.

Aqueous suspensions contain the active materials in admixture with excipients suitable for the manufacture thereof. Such excipients can be suspending agents, for example sodium carboxymethylcellulose, methylcellulose, (hydroxypropyl)methyl cellulose, sodium alginate, polyvinyl-pyrrolidone, gum tragacanth and gum acacia; dispersing or wetting agents, for example a naturally-occurring phosphatide (e.g., lecithin), or condensation products of an alkylene oxide with fatty acids (e.g., poly-oxyethylene stearate), or condensation products of ethylene oxide with long chain aliphatic alcohols (e.g., for heptdecaethyleneoxycetanol), or condensation products of ethylene oxide with partial esters derived from fatty acids and a hexitol (e.g., polyoxyethylene sorbitol monooleate), or condensation products of ethylene oxide with partial esters derived from fatty acids and hexitol anhydrides (e.g., polyethylene sorbitan monooleate). The aqueous suspensions may also contain one or more preservatives.

Oily suspensions may be formulated by suspending the active ingredient in a vegetable oil, for example, arachis oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified herein.

The pharmaceutical compositions may also be in the form of oil-in-water emulsions. The oily phase may be a vegetable oil, for example olive oil or arachis oil, or a mineral oil, for example, liquid paraffin, or mixtures of these. Suitable emulsifying agents may be naturally occurring gums, for example, gum acacia or gum tragacanth; naturally occurring phosphatides, for example, soy bean, lecithin, and esters or partial esters derived from fatty acids; hexitol anhydrides, for example, sorbitan monooleate; and condensation products of partial esters with ethylene oxide, for example, polyoxyethylene sorbitan monooleate.

The pharmaceutical compositions typically comprise a therapeutically effective amount of a compound of Formula (I), (II), a subembodiment as set forth herein, or a pharmaceutically acceptable salt thereof, and one or more pharmaceutically acceptable excipient. Suitable pharmaceutically acceptable excipients include, but are not limited to, antioxidants (e.g., ascorbic acid and sodium bisulfate), preservatives (e.g., benzyl alcohol, methyl parabens, ethyl or n-propyl, p-hydroxybenzoate), emulsifying agents, suspending agents, dispersing agents, solvents, fillers, bulking agents, detergents, buffers, vehicles, diluents, and/or adjuvants. For example, a suitable vehicle may be physiological saline solution or citrate buffered saline, possibly supplemented with other materials common in pharmaceutical compositions for parenteral administration. Neutral buffered saline or saline mixed with serum albumin are further exemplary vehicles. Those skilled in the art will readily recognize a variety of buffers that can be used in the pharmaceutical compositions and dosage forms contemplated herein. Typical buffers include, but are not limited to, pharmaceutically acceptable weak acids, weak bases, or mixtures thereof. As an example, the buffer components can be water soluble materials such as phosphoric acid, tartaric acids, lactic acid, succinic acid, citric acid, acetic acid, ascorbic acid, aspartic acid, glutamic acid, and salts thereof. Acceptable buffering agents include, for example, a Tris buffer, N-(2-Hydroxyethyl)piperazine-N′-(2-ethanesulfonic acid) (HEPES), 2-(N-Morpholino)ethanesulfonic acid (MES), 2-(N-Morpholino)ethanesulfonic acid sodium salt (MES), 3-(N-Morpholino)propanesulfonic acid (MOPS), and N-tris[Hydroxymethyl]methyl-3-aminopropanesulfonic acid (TAPS).

After a pharmaceutical composition has been formulated, it may be stored in sterile vials as a solution, suspension, gel, emulsion, solid, or dehydrated or lyophilized powder. Such formulations may be stored either in a ready-to-use form, a lyophilized form requiring reconstitution prior to use, a liquid form requiring dilution prior to use, or other acceptable form. In some embodiments, the pharmaceutical composition is provided in a single-use container (e.g., a single-use vial, ampoule, syringe, or autoinjector (similar to, e.g., an EpiPen®)), whereas a multi-use container (e.g., a multi-use vial) is provided in other embodiments.

Formulations can also include carriers to protect the composition against rapid degradation or elimination from the body, such as a controlled release formulation, including liposomes, hydrogels, prodrugs and microencapsulated delivery systems. For example, a time delay material such as glyceryl monostearate or glyceryl stearate alone, or in combination with a wax, may be employed. Any drug delivery apparatus may be used to deliver a compound of Formula (I), (II), a subembodiment as set forth herein, or a salt thereof, including implants (e.g., implantable pumps) and catheter systems, slow injection pumps and devices, all of which are well known to the skilled artisan.

Depot injections, which are generally administered subcutaneously or intramuscularly, may also be utilized to release the compound of Formula (I), (II), a subembodiment as set forth herein, or a salt thereof as set forth herein over a defined period of time. Depot injections are usually either solid- or oil-based and generally comprise at least one of the formulation components set forth herein. One of ordinary skill in the art is familiar with possible formulations and uses of depot injections.

The pharmaceutical compositions may be in the form of a sterile injectable aqueous or oleagenous suspension. The suspension may be formulated according to the known art using those suitable dispersing or wetting agents and suspending agents mentioned herein. The sterile injectable preparation may also be a sterile injectable solution or suspension in a non-toxic parenterally acceptable diluent or solvent, for example, as a solution in 1,3-butane diol. Acceptable diluents, solvents and dispersion media that may be employed include water, Ringer's solution, isotonic sodium chloride solution, Cremophor EL™ (BASF, Parsippany, NJ) or phosphate buffered saline (PBS), ethanol, polyol (e.g., glycerol, propylene glycol, and liquid polyethylene glycol), and suitable mixtures thereof. In addition, sterile, fixed oils are conventionally employed as a solvent or suspending medium. For this purpose, any bland fixed oil may be employed, including synthetic mono- or diglycerides. Moreover, fatty acids such as oleic acid, find use in the preparation of injectables. Prolonged absorption of particular injectable formulations can be achieved by including an agent that delays absorption (e.g., aluminum monostearate or gelatin).

A compound of Formula (I), (II), a subembodiment as set forth herein, or a salt thereof may also be administered in the form of suppositories for rectal administration or sprays for nasal or inhalation use. The suppositories can be prepared by mixing the drug with a suitable non-irritating excipient which is solid at ordinary temperatures but liquid at the rectal temperature and will therefore melt in the rectum to release the drug. Such materials include, but are not limited to, cocoa butter and polyethylene glycols.

Routes of Administration

Compounds of Formula (I), (II), a subembodiment as set forth herein, or a pharmaceutically acceptable salt thereof and compositions containing the same may be administered in any appropriate manner. Suitable routes of administration include oral, parenteral (e.g., intramuscular, intravenous, subcutaneous (e.g., injection or implant), intraperitoneal, intracisternal, intraarticular, intraperitoneal, intracerebral (intraparenchymal) and intracerebroventricular), nasal, vaginal, sublingual, intraocular, rectal, topical (e.g., transdermal), buccal and inhalation (nasal or oral). Depot injections, which are generally administered subcutaneously or intramuscularly, may also be utilized to administer the compounds of Formula (I), (II), a subembodiment as set forth herein, or a pharmaceutically acceptable salt thereof over a defined period of time. Particular embodiments of the present inventive concept contemplate oral administration.

Combination Therapy

The present inventive concept contemplates the use of compounds of Formula (I), (II), a subembodiment as set forth herein, or a pharmaceutically acceptable salt thereof in combination with one or more active therapeutic agents (e.g., chemotherapeutic agents) or other prophylactic or therapeutic modalities (e.g., radiation). In such combination therapy, the various active agents frequently have different, complementary mechanisms of action. Such combination therapy may be especially advantageous by allowing a dose reduction of one or more of the agents, thereby reducing or eliminating the adverse effects associated with one or more of the agents. Furthermore, such combination therapy may have a synergistic therapeutic or prophylactic effect on the underlying disease, disorder, or condition.

As used herein, “combination” is meant to include therapies that can be administered separately, for example, formulated separately for separate administration (e.g., as may be provided in a kit), and therapies that can be administered together in a single formulation (i.e., a “co-formulation”).

In certain embodiments, the compounds of Formula (I), (II), a subembodiment as set forth herein, or a pharmaceutically acceptable salt thereof are administered or applied sequentially, e.g., where one agent is administered prior to one or more other agents. In other embodiments, the compounds of Formula (I), (II), a subembodiment as set forth herein, or a pharmaceutically acceptable salt thereof are administered simultaneously, e.g., where two or more agents are administered at or about the same time; the two or more agents may be present in two or more separate formulations or combined into a single formulation (i.e., a co-formulation). Regardless of whether the two or more agents are administered sequentially or simultaneously, they are considered to be administered in combination for purposes of the present disclosure.

The compounds of Formula (I), (II), a subembodiment as set forth herein, or a pharmaceutically acceptable salt thereof may be used in combination with at least one other (active) agent in any manner appropriate under the circumstances. In one embodiment, treatment with the at least one active agent and at least one compound of Formula (I), (II), a subembodiment as set forth herein, or a pharmaceutically acceptable salt thereof is maintained over a period of time. In another embodiment, treatment with the at least one active agent is reduced or discontinued (e.g., when the subject is stable), while treatment with the compound of Formula (I), (II), a subembodiment as set forth herein, or a pharmaceutically acceptable salt thereof is maintained at a constant dosing regimen. In a further embodiment, treatment with the at least one active agent is reduced or discontinued (e.g., when the subject is stable), while treatment with a compound of Formula (I), (II), a subembodiment as set forth herein, or a pharmaceutically acceptable salt thereof is reduced (e.g., lower dose, less frequent dosing or shorter treatment regimen). In yet another embodiment, treatment with the at least one active agent is reduced or discontinued (e.g., when the subject is stable), and treatment with the compound of Formula (I), (II), a subembodiment as set forth herein, or a pharmaceutically acceptable salt thereof is increased (e.g., higher dose, more frequent dosing or longer treatment regimen). In yet another embodiment, treatment with the at least one active agent is maintained and treatment with the compound of Formula (I), (II), a subembodiment as set forth herein, or a pharmaceutically acceptable salt thereof is reduced or discontinued (e.g., lower dose, less frequent dosing or shorter treatment regimen). In yet another embodiment, treatment with the at least one active agent and treatment with the compound of Formula (I), (II), a subembodiment as set forth herein, or a pharmaceutically acceptable salt thereof are reduced or discontinued (e.g., lower dose, less frequent dosing or shorter treatment regimen).

The present disclosure provides methods for treating cancer with a compound of Formula (I), (II), a subembodiment as set forth herein, or a pharmaceutically acceptable salt thereof and at least one additional therapeutic or diagnostic agent.

In some embodiments, the compound of Formula (I), (II), a subembodiment as set forth herein, or a pharmaceutically acceptable salt thereof is administered in combination with at least one additional therapeutic agent, selected from Temozolomide, Pemetrexed, Pegylated liposomal doxorubicin (Doxil), Eribulin (Halaven), Ixabepilone (Ixempra), Protein-bound paclitaxel (Abraxane), Oxaliplatin, Irinotecan, Venatoclax (bcl2 inhibitor), 5-azacytadine, Anti-CD20 therapeutics, such as Rituxan and obinutuzumab, Hormonal agents (anastrozole, exemestand, letrozole, zoladex, lupon eligard), CDK4/6 inhibitors, Palbociclib, Abemaciclib, CPI (Avelumab, Cemiplimab-rwlc, and Bevacizumab.

In certain embodiments, the present disclosure provides methods for treating cancer including administration of a compound of Formula (I), (II), a subembodiment as set forth herein, or a salt thereof as set forth herein in combination with a signal transduction inhibitor (STI) to achieve additive or synergistic suppression of tumor growth. As used herein, the term “signal transduction inhibitor” refers to an agent that selectively inhibits one or more steps in a signaling pathway. Examples of signal transduction inhibitors (STIs) useful in methods as set forth herein include, but are not limited to: (i) bcr/abl kinase inhibitors (e.g., GLEEVEC); (ii) epidermal growth factor (EGF) receptor inhibitors, including kinase inhibitors and antibodies; (iii) her-2/neu receptor inhibitors (e.g., HERCEPTIN); (iv) inhibitors of Akt family kinases or the Akt pathway (e.g., rapamycin); (v) cell cycle kinase inhibitors (e.g., flavopiridol); and (vi) phosphatidyl inositol kinase inhibitors. Agents involved in immunomodulation can also be used in combination with one or more compounds of Formula (I), (II), a subembodiment as set forth herein, or a pharmaceutically acceptable salt thereof as set forth herein for the suppression of tumor growth in cancer patients.

In certain embodiments, the present disclosure provides methods for treating cancer including administration of a compound of Formula (I), (II), a subembodiment as set forth herein, or a pharmaceutically acceptable salt thereof as set forth herein in combination with a chemotherapeutic agents. Examples of chemotherapeutic agents include, but are not limited to, alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamime; nitrogen mustards such as chiorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, caminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogs such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; razoxane; sizofiran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (Ara-C); cyclophosphamide; thiotepa; taxoids, e.g., paclitaxel and doxetaxel; chlorambucil; gemcitabine; 6-thioguanine; mercaptopurine; methotrexate; platinum and platinum coordination complexes such as cisplatin and carboplatin; vinblastine; etoposide (VP-16); ifosfamide; mitomycin C; mitoxantrone; vincristine; vinorelbine; navelbine; novantrone; teniposide; daunomycin; aminopterin; xeloda; ibandronate; CPT11; topoisomerase inhibitors; difluoromethylornithine (DMFO); retinoic acid; esperamicins; capecitabine; PARP inhibitors such as olaparib, rucaparib, niraparib, talazoparib, veliparib, and pamiparib, DNA damage repair inhibitors such as inhibitors of ATM [such as AZ: (AZD1390) Astrazeneca's AZD0156, AZ31, AZ32; Kudos' KU-55933, KU-60019, and KU-59403; and Pfizer's CP-466722]; ATR [such as Astrazeneca's Ceralasertib (AZD6738); Repare's RP-3500; Vertex/EMD Serono's Berzosertib (VX-970/M6620); and EMD Serono's M4344; and DNA-PK (such as Astrazeneca's AZD7648; NU7441; NU7026; Kudos' KU-0060648; Vertex's VX-984; and EMD Serono's Nedisertib (M3814)] and Cyteir Therapeutics RAD51 inhibitor CYT-0851 and pharmaceutically acceptable salts, acids or derivatives of any of the above. In a particular embodiment, compounds of the present disclosure are coadministered with a cytostatic compound selected from the group consisting of cisplatin, doxorubicin, taxol, taxotere and mitomycin C. In a particular embodiment, the cytostatic compound is doxorubicin.

Chemotherapeutic agents also include anti-hormonal agents that act to regulate or inhibit hormonal action on tumors such as anti-estrogens, including for example tamoxifen, raloxifene, aromatase inhibiting 4(5)-imidazoles, 4-hydroxytamoxifen, trioxifene, keoxifene, onapristone, and toremifene; and antiandrogens such as flutamide, nilutamide, bicalutamide, enzalutamide, apalutamide, abiraterone acetate, leuprolide, and goserelin; and pharmaceutically acceptable salts, acids or derivatives of any of the above. In certain embodiments, combination therapy comprises administration of a hormone or related hormonal agent.

The present disclosure also contemplates the use of the compounds of Formula (I), (II), a subembodiment as set forth herein, or a pharmaceutically acceptable salt thereof as set forth herein in combination with immune checkpoint inhibitors. The tremendous number of genetic and epigenetic alterations that are characteristic of all cancers provides a diverse set of antigens that the immune system can use to distinguish tumor cells from their normal counterparts. In the case of T cells, the ultimate amplitude (e.g., levels of cytokine production or proliferation) and quality (e.g., the type of immune response generated, such as the pattern of cytokine production) of the response, which is initiated through antigen recognition by the T-cell receptor (TCR), is regulated by a balance between co-stimulatory and inhibitory signals (immune checkpoints). Under normal physiological conditions, immune checkpoints are crucial for the prevention of autoimmunity (i.e., the maintenance of self-tolerance) and also for the protection of tissues from damage when the immune system is responding to pathogenic infection. The expression of immune checkpoint proteins can be dysregulated by tumors as an important immune resistance mechanism. Examples of immune checkpoint inhibitors include but are not limited to CTLA-4, PD-1, PD-L1, BTLA, TIM3, LAG3, OX40, 41BB, VISTA, CD96, TGF [symbol] CD73, CD39, A2AR, A2BR, IDO1, TDO2, Arginase, B7-H3, B7-H4. Cell-based modulators of anti-cancer immunity are also contemplated. Examples of such modulators include but are not limited to chimeric antigen receptor T-cells, tumor infiltrating T-cells and dendritic-cells.

The present disclosure contemplates the use of compounds of Formula (I), (II), a subembodiment as set forth herein, or a pharmaceutically acceptable salt thereof as set forth herein in combination with inhibitors of the aforementioned immune-checkpoint receptors and ligands, for example ipilimumab, abatacept, nivolumab, pembrolizumab, atezolizumab, nivolumab, and durvalumab.

Additional treatment modalities that may be used in combination with a compound of Formula (I), (II), a subembodiment as set forth herein, or a pharmaceutically acceptable salt thereof as set forth herein include radiotherapy, a monoclonal antibody against a tumor antigen, a complex of a monoclonal antibody and toxin, a T-cell adjuvant, bone marrow transplant, or antigen presenting cells (e.g., dendritic cell therapy).

The present disclosure contemplates the use of compounds of Formula (I), (II), a subembodiment as set forth herein, or a pharmaceutically acceptable salt thereof as set forth herein for the treatment of glioblastoma either alone or in combination with radiation and/or temozolomide (TMZ), avastin or lomustine.

The present disclosure encompasses pharmaceutically acceptable salts, acids or derivatives of any of the above.

Dosing

The compounds of Formula (I), (II), a subembodiment as set forth herein, or a pharmaceutically acceptable salt thereof as set forth herein may be administered to a subject in an amount that is dependent upon, for example, the goal of administration (e.g., the degree of resolution desired); the age, weight, sex, and health and physical condition of the subject to which the formulation is being administered; the route of administration; and the nature of the disease, disorder, condition or symptom thereof. The dosing regimen may also take into consideration the existence, nature, and extent of any adverse effects associated with the agent(s) being administered. Effective dosage amounts and dosage regimens can readily be determined from, for example, safety and dose-escalation trials, in vivo studies (e.g., animal models), and other methods known to the skilled artisan.

In general, dosing parameters dictate that the dosage amount be less than an amount that could be irreversibly toxic to the subject (the maximum tolerated dose (MTD)) and not less than an amount required to produce a measurable effect on the subject. Such amounts are determined by, for example, the pharmacokinetic and pharmacodynamic parameters associated with ADME, taking into consideration the route of administration and other factors.

An effective dose (ED) is the dose or amount of an agent that produces a therapeutic response or desired effect in some fraction of the subjects taking it. The “median effective dose” or ED₅₀ of an agent is the dose or amount of an agent that produces a therapeutic response or desired effect in 50% of the population to which it is administered. Although the ED₅₀ is commonly used as a measure of reasonable expectance of an agent's effect, it is not necessarily the dose that a clinician might deem appropriate taking into consideration all relevant factors. Thus, in some situations the effective amount is more than the calculated ED₅₀, in other situations the effective amount is less than the calculated ED₅₀, and in still other situations the effective amount is the same as the calculated ED₅₀.

In addition, an effective dose of a compound of Formula (I), (II), a subembodiment as set forth herein, or a pharmaceutically acceptable salt thereof, as set forth herein, may be an amount that, when administered in one or more doses to a subject, produces a desired result relative to a healthy subject. For example, for a subject experiencing a particular disorder, an effective dose may be one that improves a diagnostic parameter, measure, marker and the like of that disorder by at least about 5%, at least about 10%, at least about 20%, at least about 25%, at least about 30%, at least about 40%, at least about 50%, at least about 60%, at least about 70%, at least about 80%, at least about 90%, or more than 90%, where 100% is defined as the diagnostic parameter, measure, marker and the like exhibited by a normal subject.

In certain embodiments, the compounds of Formula (I), (II), a subembodiment as set forth herein, or a pharmaceutically acceptable salt thereof as set forth herein may be administered (e.g., orally) at dosage levels of about 0.01 mg/kg to about 50 mg/kg, or about 1 mg/kg to about 25 mg/kg, of subject body weight per day, one or more times a day, to obtain the desired therapeutic effect.

For administration of an oral agent, the compositions can be provided in the form of tablets, capsules and the like containing from 1.0 to 1000 milligrams of the active ingredient, particularly 1.0, 3.0, 5.0, 10.0, 15.0, 20.0, 25.0, 50.0, 75.0, 100.0, 150.0, 200.0, 250.0, 300.0, 400.0, 500.0, 600.0, 750.0, 800.0, 900.0, and 1000.0 milligrams of the active ingredient.

In certain embodiments, the dosage of the compound of Formula (I), (II), a subembodiment as set forth herein, or a pharmaceutically salt thereof is contained in a “unit dosage form”. The phrase “unit dosage form” refers to physically discrete units, each unit containing a predetermined amount of the compound of Formula (I), (II), a subembodiment as set forth herein, or a pharmaceutically acceptable salt thereof, either alone or in combination with one or more additional agents, sufficient to produce the desired effect. It will be appreciated that the parameters of a unit dosage form will depend on the particular agent and the effect to be achieved.

Kits

The present inventive concept also contemplates kits including a compound of Formula (I), (II), a subembodiment as set forth herein, or a pharmaceutically acceptable salt thereof, and pharmaceutical compositions thereof. The kits are generally in the form of a physical structure housing various components, as set forth below, and may be utilized, for example, in practicing the methods as set forth above.

A kit can include one or more of the compound of Formula (I), (II), a subembodiment as set forth herein, or a pharmaceutically acceptable salt thereof as set forth herein (provided in, e.g., a sterile container), which may be in the form of a pharmaceutical composition suitable for administration to a subject. The compound of Formula (I), (II), a subembodiment as set forth herein, or a pharmaceutically acceptable salt thereof can be provided in a form that is ready for use (e.g., a tablet or capsule) or in a form requiring, for example, reconstitution or dilution (e.g., a powder) prior to administration. When the compounds of Formula (I), (II), a subembodiment as set forth herein, or a pharmaceutically acceptable salt thereof are in a form that needs to be reconstituted or diluted by a user, the kit may also include diluents (e.g., sterile water), buffers, pharmaceutically acceptable excipients, and the like, packaged with or separately from the compounds of Formula (I), (II), a subembodiment as set forth herein, or a pharmaceutically acceptable salt thereof. When combination therapy is contemplated, the kit may contain the several agents separately or they may already be combined in the kit. Each component of the kit may be enclosed within an individual container, and all of the various containers may be within a single package. A kit of the present inventive concept may be designed for conditions necessary to properly maintain the components housed therein (e.g., refrigeration or freezing).

A kit may contain a label or packaging insert including identifying information for the components therein and instructions for their use (e.g., dosing parameters, clinical pharmacology of the active ingredient(s), including mechanism of action, pharmacokinetics and pharmacodynamics, adverse effects, contraindications, etc.). Labels or inserts can include manufacturer information such as lot numbers and expiration dates. The label or packaging insert may be, e.g., integrated into the physical structure housing the components, contained separately within the physical structure, or affixed to a component of the kit (e.g., an ampule, tube or vial).

Labels or inserts can additionally include, or be incorporated into, a computer readable medium, such as a disk (e.g., hard disk, card, memory disk), optical disk such as CD- or DVD-ROM/RAM, DVD, MP3, magnetic tape, or an electrical storage media such as RAM and ROM or hybrids of these such as magnetic/optical storage media, FLASH media or memory-type cards. In some embodiments, the actual instructions are not present in the kit, but means for obtaining the instructions from a remote source, e.g., via the internet, are provided.

### EXAMPLES

The following examples and references (intermediates) are put forth so as to provide those of ordinary skill in the art with a complete disclosure and description of how to make and use the present inventive concept, and are not intended to limit the scope of what the inventors regard as their inventive concept, nor are they intended to represent that the experiments below were performed or that they are all of the experiments that may be performed. It is to be understood that exemplary descriptions written in the present tense were not necessarily performed, but rather that the descriptions can be performed to generate data and the like of a nature as set forth therein. Efforts have been made to ensure accuracy with respect to numbers used (e.g., amounts, temperature, etc.), but some experimental errors and deviations should be accounted for.

Unless indicated otherwise, parts are parts by weight, molecular weight is weight average molecular weight, temperature is in degrees Celsius (° C.), and pressure is at or near atmospheric. Standard abbreviations are used, including the following: μg=microgram; μl or μL=microliter; mM=millimolar; μM=micromolar; THIF=tetrahydrofuran; DIEA=N,N-diisopropylamine; EtOAc=ethyl acetate; TFA=trifluoroacetic acid; DCM=dichloromethane; DHP=dihydropyran; TsOH=p-Toluenesulfonic acid; FA=formic acid; TCFH=N,N,N,N′-tetramethylchloroformamidinium hexafluorophosphate; NMI=N-methylimidazole; Cs₂CO₃=cesium carbonate; XPhos Pd G3=2-dicyclohexylphosphino-2′,4′,6′-triisopropyl-1,1′-biphenyl)[2-(2′-amino-1,1′-biphenyl)]palladium-(II) methanesulfonate; LiCl=lithium chloride; POCl3=phosphoryl chloride; PE=petroleum ether; DMSO=dimethylsulfoxide; HCl=hydrochloric acid; Na₂SO₄=sodium sulfate; DMF=dimethylformamide; NaOH=sodium hydroxide; K₂CO₃=potassium carbonate; MeCN=acetonitrile; BOC=tert-butoxycarbonyl; MTBE=methyl tert-butyl ether; MeOH=methanol; NaHCO₃=sodium bicarbonate; NaBH₃CN=sodium cyanoborohydride; EtOH=ethanol; PCl₅=phosphorus pentachloride; NH₄OAc=ammonium acetate; Et₂O=ether; HOAc=acetic acid; Ac₂O=acetic anhydride; i-PrOH=isopropanol; NCS=N-chlorosuccinimide; K₃PO₄=potassium phosphate; Pd(dtbpf)Cl₂=1,1′-bis(di-tert-butylphosphino)ferrocene]-dichloropalladium(II); Pd(dppf)Cl₂=[1,1′-Bis(diphenylphosphino)ferrocene]dichloropalladium(II); Pd(dppf)Cl₂-DCM=[1,1′-Bis(diphenylphosphino)ferrocene]dichloropalladium(II), complex with dichloromethane; Zn(CN)₂=Zinc cyanide; Pd(PPh₃)₄=tetrakis(triphenylphosphine)-palladium(0); Et₃N=triethylamine; CuCN=copper cyanide; t-BuONO=tert-butyl nitrite; HATU=1-[Bis(dimethylamino)methylene]-1H-1,2,3-triazolo[4,5-b]pyridinium 3-oxid hexafluorophosphate; DBU=1,8-diazabicyclo(5.4.0)undec-7-ene; LiAlH₄=lithium aluminium hydride; NH₃=ammonia; H₂SO₄=sulfuric acid; H₂O₂=hydrogen peroxide; NMP=N-methyl-2-pyrrolidone; MgSO₄=magnesium sulphate.

### SYNTHETIC EXAMPLES

General Procedure A: Preparation of Alkyl or Aryl Amide

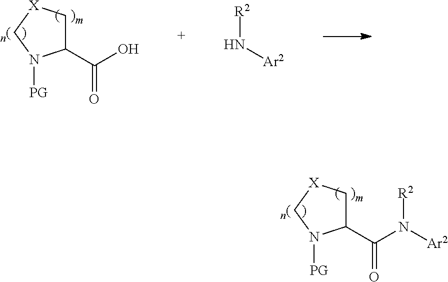

To a solution of cyclic carboxylic acid (1 eq.) where PG is an amino protecting group (e.g., Boc, Cbz and the like) and NR²Ar² (1.5 eq.) in THE (0.5M) was added DIEA (2 eq.) and propylphosphonic anhydride solution (50 wt % in EtOAc, 1.5 eq.). The resulting solution was stirred overnight at room temperature. The resulting mixture was diluted with water (30 mL). The aqueous layer was extracted with EtOAc (3×50 mL). The resulting mixture was concentrated under reduced pressure. The combined organic layers were concentrated under reduced pressure.

General Procedure B: Deprotection of Boc

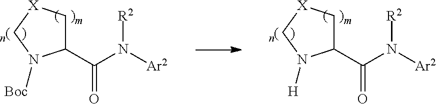

A solution of the Boc-protected cyclic amine in 25% TFA in DCM (0.2 M) was stirred at room temperature until the reaction was complete as monitored by LCMS. The mixture was concentrated under reduced pressure.

General Procedure C: Preparation of Compounds Via S_{N}Ar

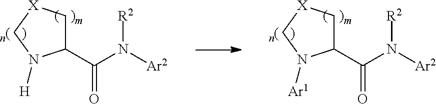

In a glass tube, purged with nitrogen and maintained under nitrogen atmosphere was placed cycloalkylamine or it's salt (HCl or TFA) (1.0 eq.), arylhalide (1 eq.), DIEA (2.0 eq.) and NMP (0.2 M) was added. The glass tube was sealed, and the reaction mixture was heated at 50° C. for 3 h. The mixture was diluted with water (5 mL) and extracted with CH₂Cl₂ (2×5 mL). The combined organic layers were dried over anhydrous sodium sulfate, filtered, concentrated under reduced pressure.

### Example 1

### Synthesis of 1-(4,6-bis(trifluoromethyl)pyridin-2-yl)-N-(4-fluorophenyl)-N-methylpyrrolidine-2-carboxamide

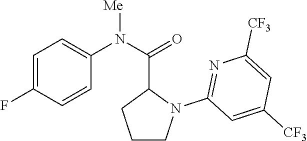 [SMILES: CN(C(=O)C1CCCN1c1cc(C(F)(F)F)cc(C(F)(F)F)n1)c1ccc(F)cc1]

### Step 1: Preparation of tert-butyl 2-((4-fluorophenyl)(methyl)carbamoyl)pyrrolidine-1-carboxylate

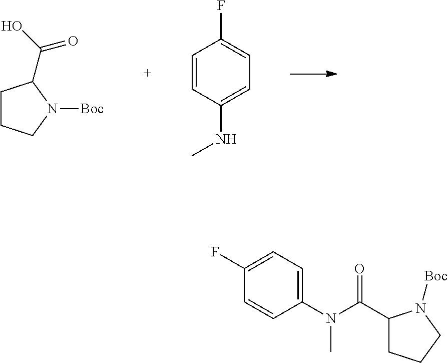

The title compound was prepared using General Procedure A, employing 1-(tert-butoxycarbonyl)pyrrolidine-2-carboxylic acid and 4-fluoro-N-methylaniline as starting materials. The residue was purified by silica gel column chromatography (eluent: 0-20% EtOAc in PE) to afford tert-butyl 2-[(4-fluorophenyl)(methyl)carbamoyl]pyrrolidine-1-carboxylate (1.10 g, 74.0%) as brown oil.

### Step 2: Preparation of N-(4-fluorophenyl)-N-methylpyrrolidine-2-carboxamide

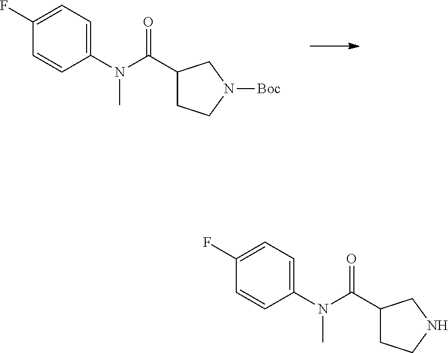 [SMILES: CN(C(=O)C1CCN(C(=O)OC(C)(C)C)C1)c1ccc(F)cc1 ; CN(C(=O)C1CCNC1)c1ccc(F)cc1]

The title compound was prepared using General Procedure B, employing tert-butyl 2-[(4-fluorophenyl)(methyl)carbamoyl]pyrrolidine-1-carboxylate as starting material. The crude product was used directly in the next step without further purification.

### Step 3: Preparation of 1-(4,6-bis(trifluoromethyl)pyridin-2-yl)-N-(4-fluorophenyl)-N-methylpyrrolidine-2-carboxamide

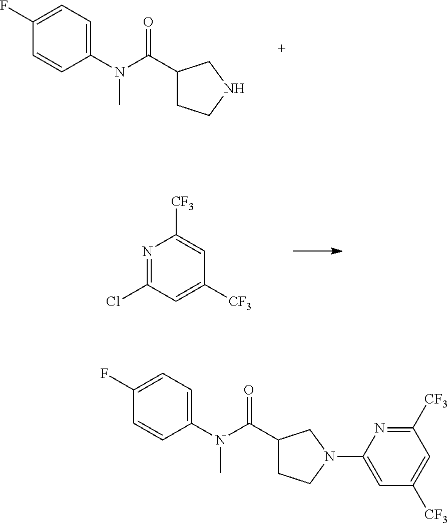 [SMILES: CN(C(=O)C1CCNC1)c1ccc(F)cc1 ; FC(F)(F)c1cc(Cl)nc(C(F)(F)F)c1 ; CN(C(=O)C1CCN(c2cc(C(F)(F)F)cc(C(F)(F)F)n2)C1)c1ccc(F)cc1]

The title compound was prepared using General Procedure C, employing N-(4-fluorophenyl)-N-methylpyrrolidine-2-carboxamide and 2-chloro-4,6-bis(trifluoromethyl)pyridine as starting materials. The residue was purified by Prep-TLC (hexane/EtOAc 3:1) to afford 1-[4,6-bis(trifluoromethyl)pyridin-2-yl]-N-(4-fluorophenyl)-N-methylpyrrolidine-2-carboxamide (22.8 mg, 23.2%) as a colorless oil. ¹H NMR (300 MHz, CDCl₃): δ (ppm) 7.45 (s, 2H), 7.27-7.13 (m, 2H), 7.07 (s, 1H), 6.75 (s, 1H), 4.55 (t, 1H), 3.76-3.69 (m, 1H), 3.53-3.47 (m, 1H), 3.26 (s, 3H), 2.36-2.27 (m, 1H), 2.09-1.97 (m, 3H). m/z 436 (M+H⁺).

### Example 2

### Synthesis of (R)-1-(4,6-bis(trifluoromethyl)pyridin-2-yl)-N-(4-fluorophenyl)-N-methylpyrrolidine-2-carboxamide

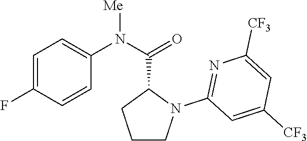 [SMILES: CN(C(=O)[C@H]1CCCN1c1cc(C(F)(F)F)cc(C(F)(F)F)n1)c1ccc(F)cc1]

### Step 1: Preparation of tert-butyl (R)-2-((4-fluorophenyl)(methyl)carbamoyl)pyrrolidine-1-carboxylate

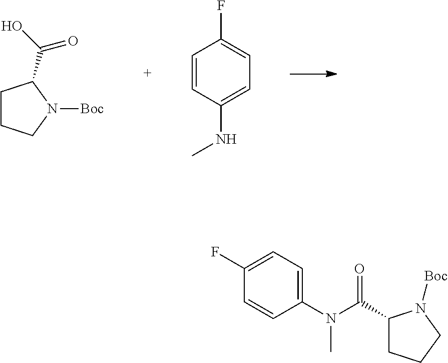

To a solution of (2R)-1-(tert-butoxycarbonyl)pyrrolidine-2-carboxylic acid (1.00 g, 4.646 mmol, 1.00 equiv) in DMF (10.00 mL) were added 4-fluoro-N-methylaniline (1.16 g, 9.269 mmol, 2.00 equiv), EDCI (1.34 g, 6.990 mmol, 1.50 equiv) and HOBT (0.75 g, 5.550 mmol, 1.19 equiv). The reaction mixture was stirred overnight at room temperature. The mixture was diluted with water (30 mL) and extracted with EtOAc (2×30 mL). The combined organic extracts were dried over anhydrous sodium sulfate, filtered and concentrated under reduced pressure. The residue was purified by silica gel column chromatography (eluent: 0-20% EtOAc in PE) to afford tert-butyl (2R)-2-[(4-fluorophenyl)(methyl)carbamoyl]pyrrolidine-1-carboxylate as an orange oil (860 mg 97.7%).

### Step 2: Preparation of (R)—N-(4-fluorophenyl)-N-methylpyrrolidine-2-carboxamide

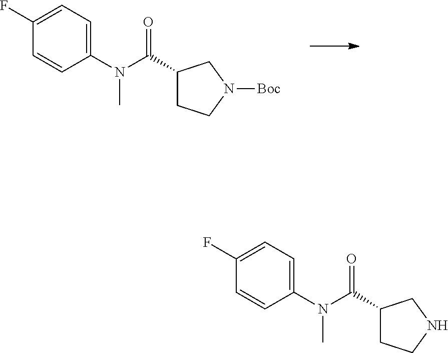 [SMILES: CN(C(=O)[C@H]1CCN(C(=O)OC(C)(C)C)C1)c1ccc(F)cc1 ; CN(C(=O)[C@H]1CCNC1)c1ccc(F)cc1]

The title compound was prepared using General Procedure B, employing tert-butyl (2R)-2-[(4-fluorophenyl)(methyl)carbamoyl]pyrrolidine-1-carboxylate as starting material. The crude product was used directly in the next step without further purification.

### Step 3: Preparation of (R)-1-(4,6-bis(trifluoromethyl)pyridin-2-yl)-N-(4-fluorophenyl)-N-methylpyrrolidine-2-carboxamide

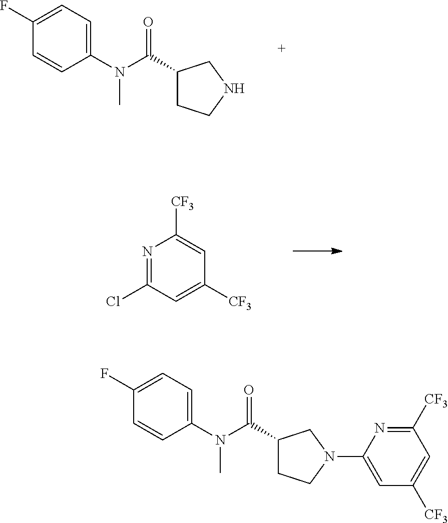 [SMILES: CN(C(=O)[C@H]1CCNC1)c1ccc(F)cc1 ; FC(F)(F)c1cc(Cl)nc(C(F)(F)F)c1 ; CN(C(=O)[C@H]1CCN(c2cc(C(F)(F)F)cc(C(F)(F)F)n2)C1)c1ccc(F)cc1]

The title compound was prepared using General Procedure C, employing (R)—N-(4-fluorophenyl)-N-methylpyrrolidine-2-carboxamide and 2-chloro-4,6-bis(trifluoromethyl)pyridine as starting materials. The residue was purified by Prep-TLC (hexane/EtOAc 3:1) to afford (R)-1-(4,6-bis(trifluoromethyl)pyridin-2-yl)-N-(4-fluorophenyl)-N-methylpyrrolidine-2-carboxamide as a white solid (11.9 mg, 32%). ¹H NMR (300 MHz, CDCl₃) δ (ppm) 7.45-7.42 (m, 2H), 7.19-7.13 (t, 2H), 7.07 (s, 1H), 6.75 (s, 1H), 4.54-4.51 (m, 1H), 3.76-3.71 (m, 1H), 3.52-3.44 (m, 1H), 3.26 (s, 3H), 2.32-2.27 (m, 1H), 2.07-1.96 (m, 3H). m/z 436 (M+H⁺).

### Example 3

### Synthesis of (S)-1-(4,6-bis(trifluoromethyl)pyridin-2-yl)-N-(4-fluorophenyl)-N-methylpyrrolidine-2-carboxamide

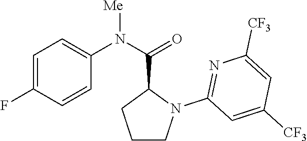 [SMILES: CN(C(=O)[C@@H]1CCCN1c1cc(C(F)(F)F)cc(C(F)(F)F)n1)c1ccc(F)cc1]

### Step 1: Preparation of tert-butyl (S)-2-((4-fluorophenyl)(methyl)carbamoyl)pyrrolidine-1-carboxylate

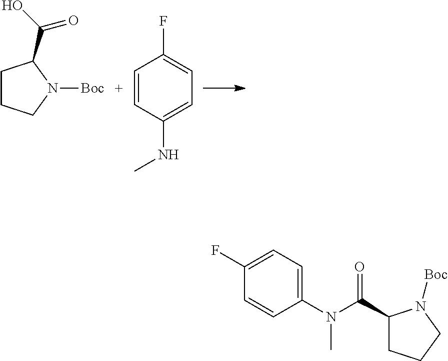

To a solution of (2S)-1-(tert-butoxycarbonyl)pyrrolidine-2-carboxylic acid (1.00 g, 4.646 mmol, 1.00 equiv) in DMF (10.00 mL) were added 4-fluoro-N-methylaniline (1.16 g, 9.269 mmol, 2.00 equiv), EDCI (1.34 g, 6.990 mmol, 1.50 equiv) and HOBT (0.75 g, 5.550 mmol, 1.19 equiv). The reaction mixture was stirred overnight at room temperature. The mixture was diluted with water (30 mL) and extracted with EtOAc (2×30 mL). The combined organic extracts were dried over anhydrous sodium sulfate, filtered and concentrated under reduced pressure. The residue was purified by silica gel column chromatography (eluent: 0-20% EtOAc in PE) to afford tert-butyl (2S)-2-[(4-fluorophenyl)(methyl)carbamoyl]pyrrolidine-1-carboxylate as an orange oil (800 mg 97%).

### Step 2: Preparation of (S)—N-(4-fluorophenyl)-N-methylpyrrolidine-2-carboxamide

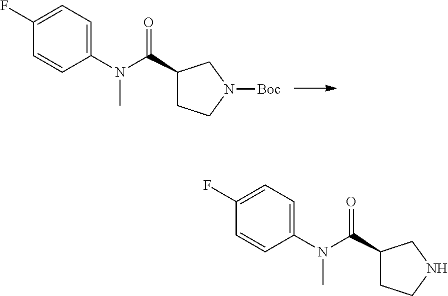 [SMILES: CN(C(=O)[C@@H]1CCN(C(=O)OC(C)(C)C)C1)c1ccc(F)cc1 ; CN(C(=O)[C@@H]1CCNC1)c1ccc(F)cc1]

The title compound was prepared using General Procedure B, employing tert-butyl (2S)-2-[(4-fluorophenyl)(methyl)carbamoyl]pyrrolidine-1-carboxylate as starting material. The crude product was used directly in the next step without further purification.

### Step 3: Preparation of (S)-1-(4,6-bis(trifluoromethyl)pyridin-2-yl)-N-(4-fluorophenyl)-N-methylpyrrolidine-2-carboxamide

The title compound was prepared using General Procedure C, employing (R)—N-(4-fluorophenyl)-N-methylpyrrolidine-2-carboxamide and 2-chloro-4,6-bis(trifluoromethyl)pyridine as starting materials. The residue was purified by Prep-TLC (hexane/EtOAc 3:1) to afford (S)-1-(4,6-bis(trifluoromethyl)pyridin-2-yl)-N-(4-fluorophenyl)-N-methylpyrrolidine-2-carboxamide as a white solid (29 mg, 37%). ¹H NMR (300 MHz, CDCl₃) δ (ppm) 7.45-7.42 (m, 2H), 7.118-7.13 (m, 2H), 7.07 (s, 1H), 6.75 (s, 1H), 4.54-4.53 (m, 1H), 3.76-3.70 (m, 1H), 3.52-3.47 (m, 1H), 3.26 (s, 3H), 2.35-2.24 (m, 1H), 2.10-1.96 (m, 3H). m/z 436 (M+H⁺)

### Example 4

### Synthesis of N-methyl-N,1-diphenylpyrrolidine-2-carboxamide

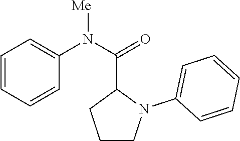 [SMILES: CN(C(=O)C1CCCN1c1ccccc1)c1ccccc1]

### Step 1: Preparation of N-methyl-N,1-diphenylpyrrolidine-2-carboxamide

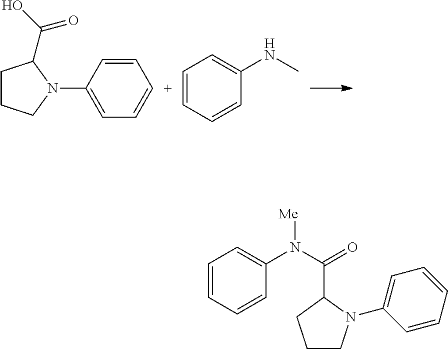

The title compound was prepared using General Procedure A, employing phenylproline and N-methylaniline as starting materials. The residue was purified by silica gel column chromatography (eluent: 0-20% EtOAc in PE) to afford N-methyl-N,1-diphenylpyrrolidine-2-carboxamide as clear oil (125 mg, 57%). ¹H NMR (400 MHz, DMSO-d₆) δ (ppm) 7.62-7.38 (m, 5H), 7.14 (t, J=7.7 Hz, 2H), 6.57 (t, J=7.3 Hz, 1H), 6.34 (d, J=7.9 Hz, 2H), 3.95 (d, J=7.8 Hz, 1H), 3.39 (d, J=8.9 Hz, 1H), 3.23 (q, J=7.8 Hz, 1H), 3.16 (s, 3H), 2.07 (d, J=8.1 Hz, 2H), 1.97-1.83 (m, 2H). m/z 281.2 (M+H⁺)

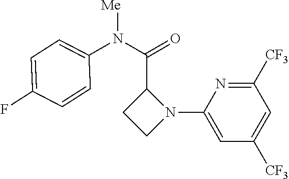 [SMILES: CN(C(=O)C1CCN1c1cc(C(F)(F)F)cc(C(F)(F)F)n1)c1ccc(F)cc1]

### Step 1: Preparation of tert-butyl 2-((4-fluorophenyl)(methyl)carbamoyl)azetidine-1-carboxylate

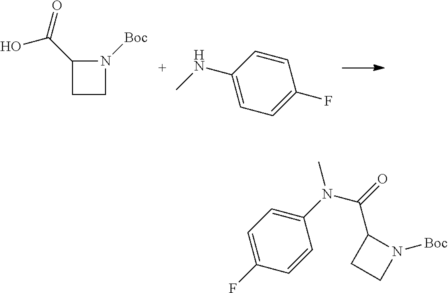

The title compound was prepared using General Procedure A, employing 1-(tert-butoxycarbonyl)azetidine-2-carboxylic acid and 4-fluoro-N-methylaniline as starting materials. The residue was purified by silica gel column chromatography (eluent: 0-20% EtOAc in PE) to afford tert-butyl 2-((4-fluorophenyl)(methyl)carbamoyl)azetidine-1-carboxylate as yellow oil (68 mg, 44%).

### Step 2: Preparation of N-(4-fluorophenyl)-N-methylazetidine-2-carboxamide

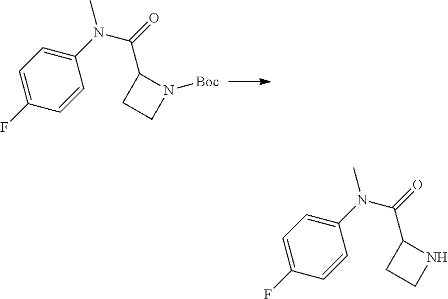 [SMILES: CN(C(=O)C1CCN1C(=O)OC(C)(C)C)c1ccc(F)cc1 ; CN(C(=O)C1CCN1)c1ccc(F)cc1]

The title compound was prepared using General Procedure B, employing tert-butyl 2-((4-fluorophenyl)(methyl)carbamoyl)azetidine-1-carboxylate as starting material. The crude product was used directly in the next step without further purification.

### Step 3: Preparation of 1-(4,6-bis(trifluoromethyl)pyridin-2-yl)-N-(4-fluorophenyl)-N-methylazetidine-2-carboxamide

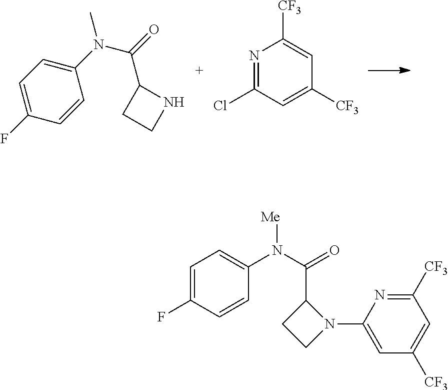 [SMILES: CN(C(=O)C1CCN1)c1ccc(F)cc1 ; FC(F)(F)c1cc(Cl)nc(C(F)(F)F)c1 ; CN(C(=O)C1CCN1c1cc(C(F)(F)F)cc(C(F)(F)F)n1)c1ccc(F)cc1]

The title compound was prepared using General Procedure C, employing N-(4-fluorophenyl)-N-methylazetidine-2-carboxamide and 2-chloro-4,6-bis(trifluoromethyl)pyridine as starting materials. The residue was purified by prep-HPLC.

The pure fractions were combined and lyophilized to afford 1-(4,6-bis(trifluoromethyl)pyridin-2-yl)-N-(4-fluorophenyl)-N-methylazetidine-2-carboxamide (12 mg, 10%) as a white solid. ¹H NMR (400 MHz, Chloroform-d) δ (ppm) 7.34 (t, J=6.5 Hz, 2H), 7.13 (t, J=8.3 Hz, 2H), 7.07 (s, 1H), 6.58 (s, 1H), 4.78 (t, J=6.7 Hz, 1H), 4.28 (q, J=7.2 Hz, 1H), 3.93 (q, J=7.4 Hz, 1H), 3.29 (d, J=1.9 Hz, 3H), 2.55-2.41 (m, 1H), 2.41-2.26 (m, 1H). m/z 422.2 (M+H⁺).

### Example 6

### Synthesis of 4-(4,6-bis(trifluoromethyl)pyridin-2-yl)-N-(4-fluorophenyl)-N-methylmorpholine-3-carboxamide

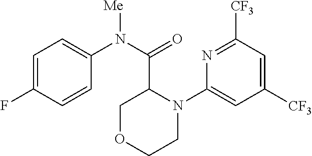 [SMILES: CN(C(=O)C1COCCN1c1cc(C(F)(F)F)cc(C(F)(F)F)n1)c1ccc(F)cc1]

### Step 1: Preparation of tert-butyl 3-((4-fluorophenyl)(methyl)carbamoyl)morpholine-4-carboxylate

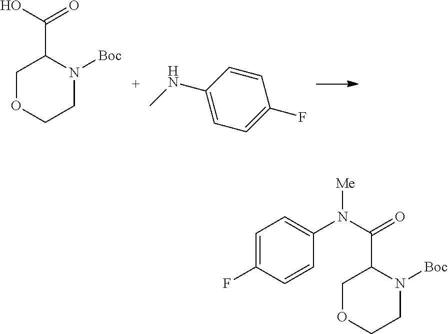

The title compound was prepared using General Procedure A, employing 4-(tert-butoxycarbonyl)morpholine-3-carboxylic acid and 4-fluoro-N-methylaniline as starting materials. The residue was purified by silica gel column chromatography (eluent: 0-50% EtOAc in PE) to afford tert-butyl 3-((4-fluorophenyl)(methyl)carbamoyl)morpholine-4-carboxylate as yellow oil (193 mg, 66%).

### Step 2: Preparation of N-(4-fluorophenyl)-N-methylmorpholine-3-carboxamide

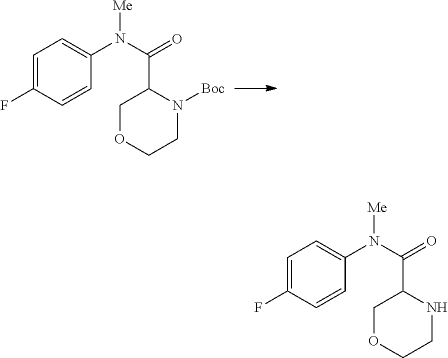 [SMILES: CN(C(=O)C1COCCN1C(=O)OC(C)(C)C)c1ccc(F)cc1 ; CN(C(=O)C1COCCN1)c1ccc(F)cc1]

The title compound was prepared using General Procedure B, employing tert-butyl 2-((4-fluorophenyl)(methyl)carbamoyl)azetidine-1-carboxylate as starting material. The crude product was used directly in the next step without further purification.

### Step 3: Preparation of 4-(4,6-bis(trifluoromethyl)pyridin-2-yl)-N-(4-fluorophenyl)-N-methylmorpholine-3-carboxamide

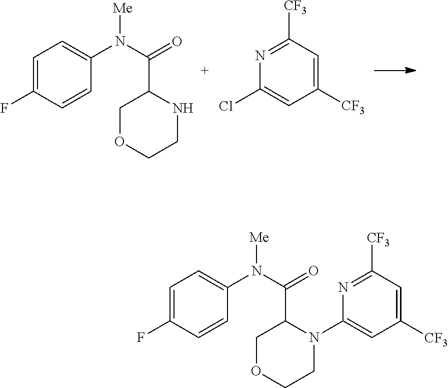 [SMILES: CN(C(=O)C1COCCN1)c1ccc(F)cc1 ; FC(F)(F)c1cc(Cl)nc(C(F)(F)F)c1 ; CN(C(=O)C1COCCN1c1cc(C(F)(F)F)cc(C(F)(F)F)n1)c1ccc(F)cc1]

The title compound was prepared using General Procedure C, employing N-(4-fluorophenyl)-N-methylmorpholine-3-carboxamide and 2-chloro-4,6-bis(trifluoromethyl)pyridine as starting materials. The residue was purified by prep-HPLC. The pure fractions were combined and lyophilized to afford 4-(4,6-bis(trifluoromethyl)pyridin-2-yl)-N-(4-fluorophenyl)-N-methylmorpholine-3-carboxamide (5 mg, 4%) as a white solid. ¹H NMR (400 MHz, CDCl₃) δ (ppm) 7.53 (s, 2H), 7.30 (d, J=1.8 Hz, 1H), 7.21 (t, J=8.4 Hz, 2H), 7.09 (s, 1H), 5.36-5.24 (m, 1H), 4.13 (ddd, J=32.6, 14.8, 8.0 Hz, 3H), 3.65 (t, J=11.0 Hz, 1H), 3.58-3.48 (m, 2H), 3.27 (s, 3H). m/z 452 (M+H⁺)

### Example 7

### Synthesis of 1-(4,6-bis(trifluoromethyl)pyridin-2-yl)-N-(4-fluorophenyl)-N-methylpiperidine-2-carboxamide

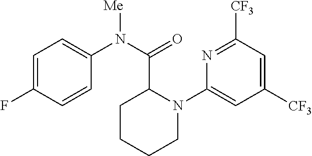 [SMILES: CN(C(=O)C1CCCCN1c1cc(C(F)(F)F)cc(C(F)(F)F)n1)c1ccc(F)cc1]

### Step 1: Preparation of tert-butyl tert-butyl 2-((4-fluorophenyl)(methyl)carbamoyl)piperidine-1-carboxylate

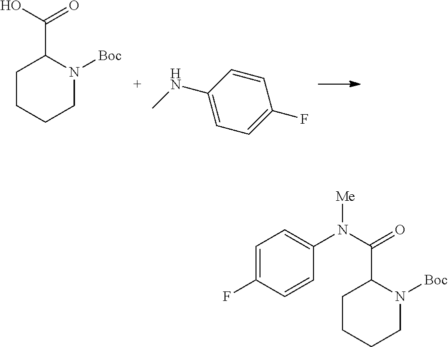

The title compound was prepared using General Procedure A, employing 1-(tert-butoxycarbonyl)piperidine-2-carboxylic acid and 4-fluoro-N-methylaniline as starting materials. The residue was purified by silica gel column chromatography (eluent: 0-20% EtOAc in PE) to afford tert-butyl 2-((4-fluorophenyl)(methyl)carbamoyl)piperidine-1-carboxylate as yellow oil (158 mg, 54%).

### Step 2: Preparation of N-(4-fluorophenyl)-N-methylpiperidine-2-carboxamide

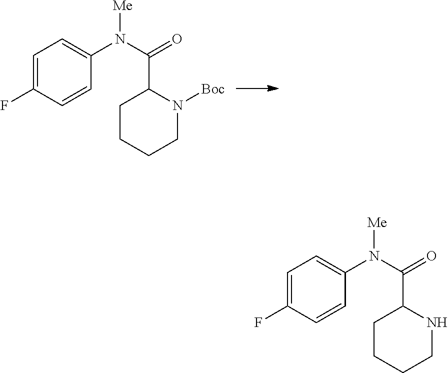 [SMILES: CN(C(=O)C1CCCCN1C(=O)OC(C)(C)C)c1ccc(F)cc1 ; CN(C(=O)C1CCCCN1)c1ccc(F)cc1]

The title compound was prepared using General Procedure B, employing tert-butyl 2-((4-fluorophenyl)(methyl)carbamoyl)piperidine-1-carboxylate as starting material. The crude product was used directly in the next step without further purification.

### Step 3: Preparation of 1-(4,6-bis(trifluoromethyl)pyridin-2-yl)-N-(4-fluorophenyl)-N-methylpiperidine-2-carboxamide

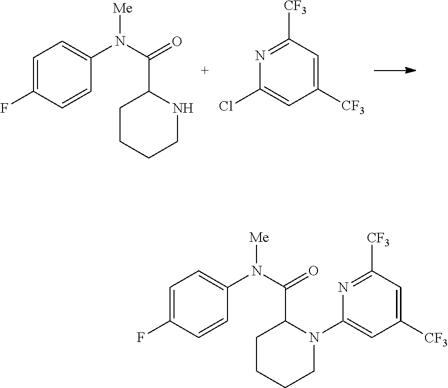 [SMILES: CN(C(=O)C1CCCCN1)c1ccc(F)cc1 ; FC(F)(F)c1cc(Cl)nc(C(F)(F)F)c1 ; CN(C(=O)C1CCCCN1c1cc(C(F)(F)F)cc(C(F)(F)F)n1)c1ccc(F)cc1]

The title compound was prepared using General Procedure C, employing N-(4-fluorophenyl)-N-methylpiperidine-2-carboxamide and 2-chloro-4,6-bis(trifluoromethyl)pyridine as starting materials. The residue was purified by prep-HPLC. The pure fractions were combined and lyophilized to afford 1-(4,6-bis(trifluoromethyl)pyridin-2-yl)-N-(4-fluorophenyl)-N-methylpiperidine-2-carboxamide (5 mg, 3.5%) as a white solid. ¹H NMR (400 MHz, CDCl₃) δ (ppm) 7.52-7.40 (m, 2H), 7.13 (d, J=11.0 Hz, 3H), 7.03 (s, 1H), 5.47 (d, J=6.2 Hz, 1H), 3.77 (dd, J=13.8, 5.2 Hz, 2H), 3.20 (s, 3H), 1.95-1.80 (m, 2H), 1.67-1.38 (m, 4H). m/z 450.3 (M+H⁺).

### Example 8

### Synthesis of 2-(2-(3-(4-fluorophenyl)pyridin-2-yl)pyrrolidin-1-yl)-4,6-bis(trifluoromethyl)pyridine

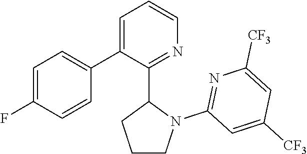 [SMILES: Fc1ccc(-c2cccnc2C2CCCN2c2cc(C(F)(F)F)cc(C(F)(F)F)n2)cc1]

### Step 1: Preparation of 3-(3-bromopicolinoyl)-1-vinylpyrrolidin-2-one

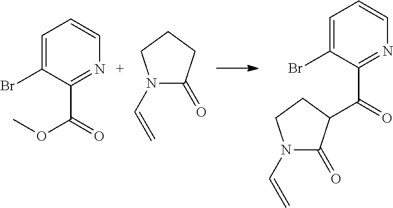

To a solution of NaH (0.44 g, 18.33 mmol, 2.0 eq.) in THE (20 mL) at 0° C. were added methyl 3-bromopyridine-2-carboxylate (2.00 g, 9.258 mmol, 1.00 equiv) and N-vinylpyrrolidone (1.13 g, 10.184 mmol, 1.10 equiv). The mixture was warmed up to RT and stirred for 30 min. The resulting mixture was heated to 60° C. and stirred for 12 h. The mixture was cooled down to room temperature and acidified with HCl (1 M). The aqueous layer was extracted with EtOAc (2×20 mL) and the combined organic extracts were dried over anhydrous sodium sulfate, filtered and concentrated under reduced pressure to afford crude product (3.1 g) as a yellow green oil.

### Step 2: Preparation of 3-bromo-2-(3,4-dihydro-2H-pyrrol-5-yl)pyridine

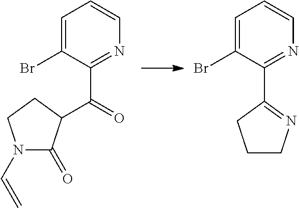 [SMILES: C=CN1CCC(C(=O)c2ncccc2Br)C1=O ; Brc1cccnc1C1=NCCC1]

To a solution of 3-(3-bromopyridine-2-carbonyl)-1-ethenylpyrrolidin-2-one (crude 3.10 g) in H₂O (7.00 mL) was added conc. HCl (4.73 mL). The reaction mixture was heated to 110° C. and stirred for 24 h. The mixture was neutralized to pH 7 using aq. NaOH (1 M) and the aqueous layer was extracted with EtOAc (3×100 mL). The combined organic extracts were dried over anhydrous sodium sulfate, filtered and concentrated under reduced pressure. The residue was purified by silica gel column chromatography (eluent: 04% EtOAc in PE) to afford 3-bromo-2-(4,5-dihydro-3H-pyrrol-2-yl)pyridine (230 mg) as a light yellow solid.

### Step 3: Preparation of 3-bromo-2-(pyrrolidin-2-yl)pyridine

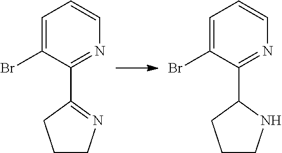 [SMILES: Brc1cccnc1C1=NCCC1 ; Brc1cccnc1C1CCCN1]

To a solution of 3-bromo-2-(4,5-dihydro-3H-pyrrol-2-yl)pyridine (100.00 mg, 0.444 mmol, 1.00 equiv) in MeOH (4.00 mL) at room temperature was added NaBH₄ (33.62 mg, 0.889 mmol, 2.00 equiv). The resulting mixture was stirred at room temperature for 30 min. The reaction was quenched with sat. aq. NaHCO₃ (10 mL) and extracted with EtOAc (3×10 mL). The combined organic extracts were dried over anhydrous sodium sulfate, filtered and concentrated under reduced pressure. The residue was purified by Prep-TLC (CH₂Cl₂/MeOH 15:1) to afford 3-bromo-2-(pyrrolidin-2-yl)pyridine (64 mg, 63%) as a white solid.

### Step 4: Preparation of 2-(2-(3-bromopyridin-2-yl)pyrrolidin-1-yl)-4,6-bis(trifluoromethyl)pyridine

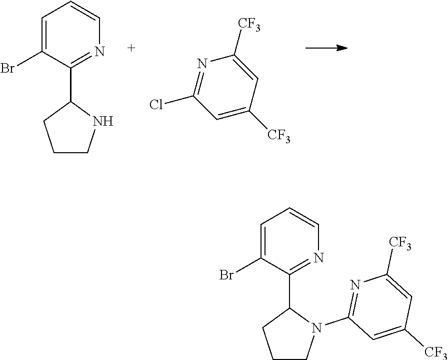 [SMILES: Brc1cccnc1C1CCCN1 ; FC(F)(F)c1cc(Cl)nc(C(F)(F)F)c1 ; FC(F)(F)c1cc(N2CCCC2c2ncccc2Br)nc(C(F)(F)F)c1]

The title compound was prepared using General Procedure C, employing 3-bromo-2-(pyrrolidin-2-yl)pyridine and 2-chloro-4,6-bis(trifluoromethyl)pyridine as starting materials. The residue was purified by Prep-TLC (hexane/EtOAc 3:1) to afford 2-[2-(3-bromopyridin-2-yl)pyrrolidin-1-yl]-4,6-bis(trifluoromethyl)pyridine (60 mg, 48%) as a white solid.

### Step 5: Preparation of 2-(2-(3-(4-fluorophenyl)pyridin-2-yl)pyrrolidin-1-yl)-4,6-bis(trifluoromethyl)pyridine

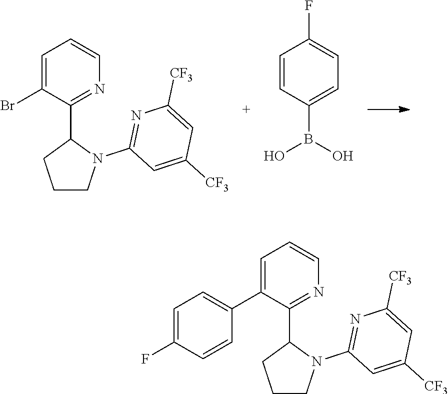 [SMILES: FC(F)(F)c1cc(N2CCCC2c2ncccc2Br)nc(C(F)(F)F)c1 ; OB(O)c1ccc(F)cc1 ; Fc1ccc(-c2cccnc2C2CCCN2c2cc(C(F)(F)F)cc(C(F)(F)F)n2)cc1]

To a solution of 2-[2-(3-bromopyridin-2-yl)pyrrolidin-1-yl]-4,6-bis(trifluoromethyl)pyridine (55.00 mg, 0.125 mmol, 1.00 equiv) in dioxane (2.00 mL) and H₂O (0.50 mL) were added K₂CO₃ (34.54 mg, 0.250 mmol, 2.00 equiv) followed by 4-fluorophenylboronic acid (26.22 mg, 0.187 mmol, 1.50 equiv). Nitrogen was bubbled through the solution for 5 min and Pd(dppf)Cl₂ (13.71 mg, 0.019 mmol, 0.15 equiv) was added. The reaction mixture was heated to 50° C. and stirred for 2 h. The mixture was diluted with EtOAc (20 mL) and washed with water (3×10 mL). The organic layer was dried over anhydrous sodium sulfate, filtered and concentrated under reduced pressure. The residue was purified by Prep-TLC (PE/EtOAc 2:1) to afford 2-[2-[3-(4-fluorophenyl)pyridin-2-yl]pyrrolidin-1-yl]-4,6-bis(trifluoromethyl)pyridine (38.5 mg, 67%) as a white solid. ¹H NMR (300 MHz, DMSO-d₆): δ (ppm) 8.40 (s, 1H), 7.60-7.48 (m, 3H), 7.37-7.28 (m, 3H), 7.04 (s, 2H), 5.20 (s, 1H), 3.79-3.78 (m, 1H), 3.66 (s, 1H), 2.28-2.27 (m, 2H), 2.00-1.90 (m, 2H). m/z 456 (M+H⁺).

### Example 9

### Synthesis of (S)-1-(4,6-bis(trifluoromethyl)pyridin-2-yl)-N-(4-bromophenyl)-N-methylpyrrolidine-2-carboxamide

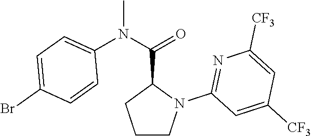 [SMILES: CN(C(=O)[C@@H]1CCCN1c1cc(C(F)(F)F)cc(C(F)(F)F)n1)c1ccc(Br)cc1]

### Step 1: Preparation of tert-butyl (4,6-bis(trifluoromethyl)pyridin-2-yl)-L-prolinate

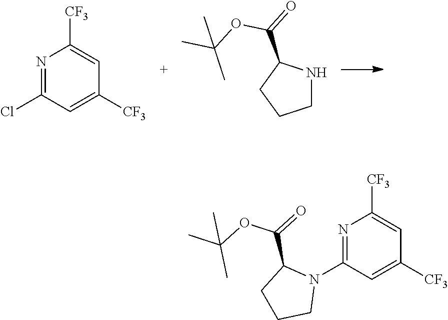 [SMILES: FC(F)(F)c1cc(Cl)nc(C(F)(F)F)c1 ; CC(C)(C)OC(=O)[C@@H]1CCCN1 ; CC(C)(C)OC(=O)[C@@H]1CCCN1c1cc(C(F)(F)F)cc(C(F)(F)F)n1]

The title compound was prepared using General Procedure C, employing tert-butyl pyrrolidine-2-carboxylate and 2-chloro-4,6-bis(trifluoromethyl)pyridine as starting materials. The residue was purified by silica gel column chromatography (eluent: 0-40% EtOAc in hexanes) to afford resulted in tert-butyl (4,6-bis(trifluoromethyl)pyridin-2-yl)-L-prolinate (890 mg, 54%) as a colorless oil.

### Step 2: Preparation of (4,6-bis(trifluoromethyl)pyridin-2-yl)-L-proline

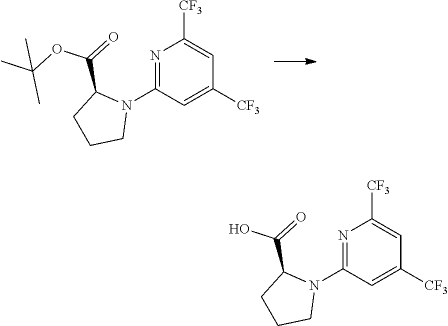 [SMILES: CC(C)(C)OC(=O)[C@@H]1CCCN1c1cc(C(F)(F)F)cc(C(F)(F)F)n1 ; O=C(O)[C@@H]1CCCN1c1cc(C(F)(F)F)cc(C(F)(F)F)n1]

The title compound was prepared using General Procedure B, employing tert-butyl (4,6-bis(trifluoromethyl)pyridin-2-yl)-L-prolinate as starting material. The residue was purified by silica gel column chromatography (eluent: 0-2% MeOH in DCM) to afford (4,6-bis(trifluoromethyl)pyridin-2-yl)-L-proline (600 mg, 77%) as a colorless oil.

### Step 3: Preparation of(S)-1-(4,6-bis(trifluoromethyl)pyridin-2-yl)-N-(4-bromophenyl)-N-methylpyrrolidine-2-carboxamide

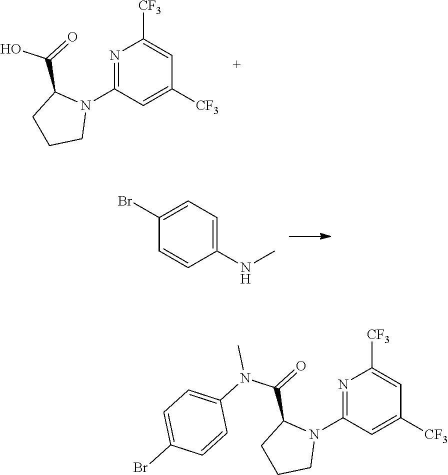

The title compound was prepared using General Procedure A, (2S)-1-[4,6-bis(trifluoromethyl)pyridin-2-yl]pyrrolidine-2-carboxylic acid and 4-bromo-N-methylaniline as starting materials. The residue was purified by Prep-TLC (PE/EtOAc 5:1) to afford (2S)-1-[4,6-bis(trifluoromethyl)pyridin-2-yl]-N-(4-bromophenyl)-N-methylpyrrolidine-2-carboxamide (50 mg, 33%) as an off-white solid. ¹H NMR (300 MHz, DMSO-d₆): δ (ppm) 7.72-7.69 (d, 2H), 7.44-7.41 (d, 2H), 7.23 (s, 1H), 7.08 (s, 1H), 4.38 (s, 1H), 3.62-3.53 (m, 2H), 3.11 (s, 3H), 2.08-1.95 (m, 4H). m/z 496 (M+H⁺).

### Example 10

### Synthesis of (S)—N-(benzofuran-5-yl)-1-(4,6-bis(trifluoromethyl)pyridin-2-yl)-N-methylpyrrolidine-2-carboxamide

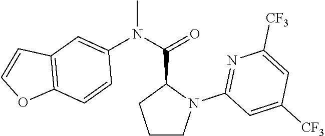 [SMILES: CN(C(=O)[C@@H]1CCCN1c1cc(C(F)(F)F)cc(C(F)(F)F)n1)c1ccc2occc2c1]

### Step 1: Preparation of (S)—N-(benzofuran-5-yl)-1-(4,6-bis(trifluoromethyl)pyridin-2-yl)-N-methylpyrrolidine-2-carboxamide

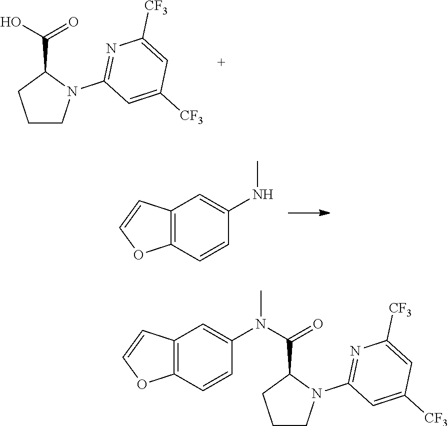 [SMILES: O=C(O)[C@@H]1CCCN1c1cc(C(F)(F)F)cc(C(F)(F)F)n1 ; CNc1ccc2occc2c1 ; CN(C(=O)[C@@H]1CCCN1c1cc(C(F)(F)F)cc(C(F)(F)F)n1)c1ccc2occc2c1]

To a stirred solution (S)-1-(4,6-bis(trifluoromethyl)pyridin-2-yl)pyrrolidine-2-carboxylic acid (100.00 mg, 0.288 mmol, 1.00 equiv) in DCM (1.00 mL) was added (CO)₂Cl₂ (43.89 mg, 0.345 mmol, 1.20 equiv) followed by DMF (2.00 mg, 0.028 mmol, 1.00 equiv). The resulting mixture was stirred for 2 h at 0° C. To the reaction mixture, N-methyl-1-benzofuran-5-amine (42.46 mg, 0.288 mmol, 1.00 equiv) and Et₃N (58.38 mg, 0.577 mmol, 2.00 equiv) were added. The resulting mixture was stirred overnight at RT. The reaction was quenched by adding EtOAc (20 mL) and the resulting mixture was washed with water (2×10 mL). The organic layer was dried over anhydrous sodium sulfate, filtered and concentrated under reduced pressure. The residue was purified by Prep-TLC (PE/EtOAc 5:1) to afford N-(1-benzofuran-5-yl)-1-[4,6-bis(trifluoromethyl)pyridin-2-yl]-N-methylpyrrolidine-2-carboxamide (20.7 mg, 16%) as a white solid. ¹H NMR (300 MHz, DMSO-d₆): δ (ppm) 8.10-8.08 (d, 1H), 7.77-7.71 (m, 2H), 7.43-7.40 (d, 1H), 7.21 (s, 1H), 7.05 (s, 1H), 6.96 (s, 2H), 4.40-4.37 (m, 1H), 3.58-3.50 (m, 1H), 3.15 (s, 3H), 2.27-1.91 (m, 4H) ppm. m/z 458 (M+H⁺).

### Example 11

### Synthesis of (S)-1-(4,6-bis(trifluoromethyl)pyridin-2-yl)-N-methyl-N-(pyridin-4-yl)pyrrolidine-2-carboxamide

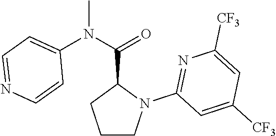 [SMILES: CN(C(=O)[C@@H]1CCCN1c1cc(C(F)(F)F)cc(C(F)(F)F)n1)c1ccncc1]

### Step 1: Preparation of (S)-1-(4,6-bis(trifluoromethyl)pyridin-2-yl)-N-methyl-N-(pyridin-4-yl)pyrrolidine-2-carboxamide

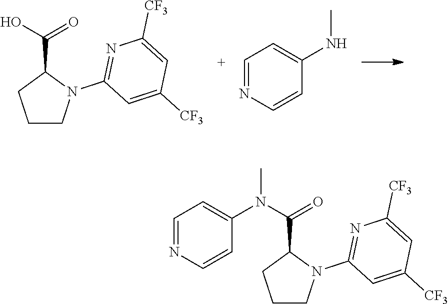

To a stirred solution (S)-1-(4,6-bis(trifluoromethyl)pyridin-2-yl)pyrrolidine-2-carboxylic acid (100.00 mg, 0.288 mmol, 1.00 equiv) in DCM (1.00 mL) was added (CO)₂Cl₂ (43.89 mg, 0.345 mmol, 1.20 equiv) followed by DMF (2.00 mg, 0.028 mmol, 1.00 equiv). The resulting mixture was stirred for 2 h at 0° C. To the reaction mixture, N-methylpyridin-4-amine (31.20 mg, 0.288 mmol, 1.00 equiv) and Et₃N (58.38 mg, 0.577 mmol, 2.00 equiv) were added. The resulting solution was heated to 40° C. and stirred overnight. The reaction mixture was diluted with water (10 mL) and the aqueous layer was extracted with EtOAc (2×30 mL). The combined organic extracts were dried over anhydrous sodium sulfate, filtered and concentrated under reduced pressure. The residue was purified by Prep-TLC (CH₂Cl₂/MeOH 40:1) to afford (2S)-1-[4,6-bis(trifluoromethyl)pyridin-2-yl]-N-methyl-N-(pyridin-4-yl)pyrrolidine-2-carboxamide (31.2 mg, 26%) as a white solid. ¹H NMR (300 MHz, DMSO-d₆): δ (ppm) 8.64-8.66 (d, 2H), 7.46-7.44 (d, 2H), 7.24 (s, 1H), 7.12 (s, 1H), 4.66 (s, 1H), 3.63-3.58 (m, 2H), 3.27 (s, 3H), 2.23-1.92 (s, 4H). m/z 419 (M+H⁺).

### Example 12

### Synthesis of (S)-1-(4,6-bis(trifluoromethyl)pyridin-2-yl)-N-cyclopropyl-N-(4-fluorophenyl)pyrrolidine-2-carboxamide

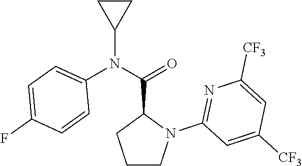 [SMILES: O=C([C@@H]1CCCN1c1cc(C(F)(F)F)cc(C(F)(F)F)n1)N(c1ccc(F)cc1)C1CC1]

### Step 1: Preparation of benzyl (S)-2-(cyclopropyl(4-fluorophenyl)carbamoyl)pyrrolidine-1-carboxylate

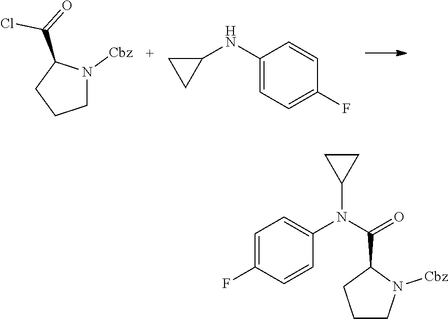 [SMILES: O=C(Cl)[C@@H]1CCCN1C(=O)OCc1ccccc1 ; Fc1ccc(NC2CC2)cc1 ; O=C([C@@H]1CCCN1C(=O)OCc1ccccc1)N(c1ccc(F)cc1)C1CC1]

To a solution of benzyl 2-(carboxy)pyrrolidine-1-carboxylate (500.00 mg, 1.868 mmol, 1.00 equiv) in DCM (5.00 mL) at room temperature was added N-cyclopropyl-4-fluoroaniline (282.37 mg, 1.868 mmol, 1.00 equiv) followed by Et₃N (377.98 mg, 3.735 mmol, 2 equiv). The resulting mixture was stirred at room temperature for 1 h. The reaction mixture was diluted with EtOAc (20 mL) and washed with water (10 mL). The organic layer was dried over anhydrous sodium sulfate, filtered and concentrated under reduced pressure. The residue was purified by Prep-TLC (CH₂Cl₂/MeOH 50:1) to afford benzyl (2S)-2-[cyclopropyl(4-fluorophenyl)carbamoyl]pyrrolidine-1-carboxylate (410 mg, 57%) as a yellow oil.

### Step 2: Preparation of (S)—N-cyclopropyl-N-(4-fluorophenyl)pyrrolidine-2-carboxamide

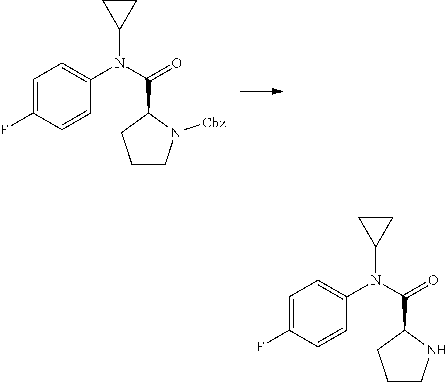 [SMILES: O=C([C@@H]1CCCN1C(=O)OCc1ccccc1)N(c1ccc(F)cc1)C1CC1 ; O=C([C@@H]1CCCN1)N(c1ccc(F)cc1)C1CC1]

To a solution of benzyl (2S)-2-[cyclopropyl(4-fluorophenyl)carbamoyl]pyrrolidine-1-carboxylate (200.00 mg, 0.523 mmol, 1.00 equiv) in MeOH (10.00 mL) at room temperature was added Pd/C (30.00 mg). The resulting mixture was stirred at room temperature for 2.5 h under a balloon of hydrogen gas. The solids were filtered off and washed with MeOH (5 mL). The filtrate was concentrated and purified by Prep-TLC (CH₂Cl₂/MeOH 15:1) to afford (2S)—N-cyclopropyl-N-(4-fluorophenyl)pyrrolidine-2-carboxamide (74 mg, 57%) as a white solid.

### Step 3: Preparation of(S)-1-(4,6-bis(trifluoromethyl)pyridin-2-yl)-N-cyclopropyl-N-(4-fluorophenyl)pyrrolidine-2-carboxamide

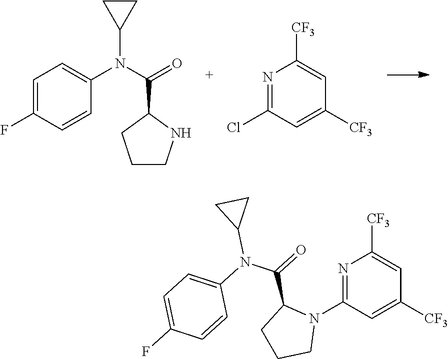 [SMILES: O=C([C@@H]1CCCN1)N(c1ccc(F)cc1)C1CC1 ; FC(F)(F)c1cc(Cl)nc(C(F)(F)F)c1 ; O=C([C@@H]1CCCN1c1cc(C(F)(F)F)cc(C(F)(F)F)n1)N(c1ccc(F)cc1)C1CC1]

The title compound was prepared using General Procedure C, employing (2S)—N-cyclopropyl-N-(4-fluorophenyl)pyrrolidine-2-carboxamide and 2-chloro-4,6-bis(trifluoromethyl)pyridine as starting materials. The residue was purified by Prep-TLC (PE/EtOAc 2:1) to afford (2S)-1-[4,6-bis(trifluoromethyl)pyridin-2-yl]-N-cyclopropyl-N-(4-fluorophenyl)pyrrolidine-2-carboxamide (31.3 mg, 42%) as a white solid. ¹H NMR (300 MHz, DMSO-d₆): δ (ppm) 7.37-7.33 (m, 2H), 7.24-7.11 (m, 4H), 5.50 (s, 0.5H), 4.25 (s, 0.5H), 3.62 (s, 2H), 3.08 (s, 1H), 2.12-1.92 (m, 4H), 1.24-0.25 (m, 4H). m/z 462 (M+H⁺).

### Example 13

### Synthesis of (2S,4S)-1-(4,6-bis(trifluoromethyl)pyridin-2-yl)-N-(4-fluorophenyl)-4-hydroxy-N-methylpyrrolidine-2-carboxamide

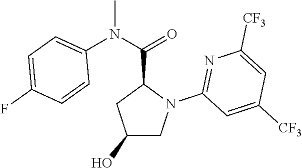 [SMILES: CN(C(=O)[C@@H]1C[C@H](O)CN1c1cc(C(F)(F)F)cc(C(F)(F)F)n1)c1ccc(F)cc1]

### Step 1: Preparation of benzyl tert-butyl (2S,4S)-2-((4-fluorophenyl)(methyl)carbamoyl)-4-hydroxypyrrolidine-1-carboxylate

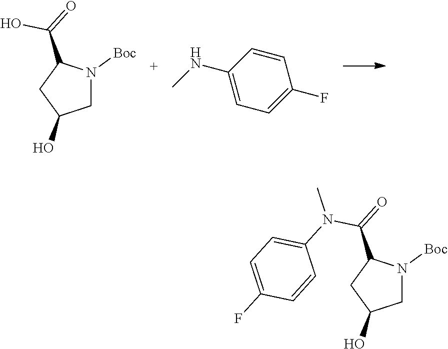

The title compound was prepared using General Procedure A, employing (2S,4S)-1-(tert-butoxycarbonyl)-4-hydroxypyrrolidine-2-carboxylic acid and 4-fluoro-N-methylaniline as starting materials. The residue was purified by Prep-TLC (CH₂Cl₂/MeOH 40:1) to afford tert-butyl (2S,4S)-2-[(4-fluorophenyl)(methyl)carbamoyl]-4-hydroxypyrrolidine-1-carboxylate (120 mg, 8%) as a brown oil.

### Step 2: Preparation of (2S,4S)—N-(4-fluorophenyl)-4-hydroxy-N-methylpyrrolidine-2-carboxamide

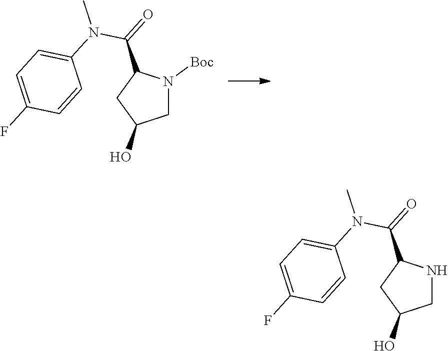

The title compound was prepared using General Procedure B, employing tert-butyl (2S,4S)-2-[(4-fluorophenyl)(methyl)carbamoyl]-4-hydroxypyrrolidine-1-carboxylate as starting material. The crude product was used directly in the next step without further purification.

### Step 3: Preparation of (2S,4S)-1-(4,6-bis(trifluoromethyl)pyridin-2-yl)-N-(4-fluorophenyl)-4-hydroxy-N-methylpyrrolidine-2-carboxamide

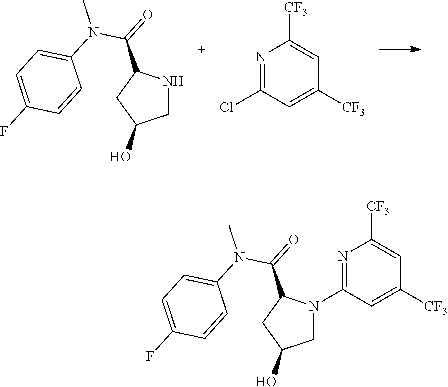

The title compound was prepared using General Procedure C, employing (2S,4S)—N-(4-fluorophenyl)-4-hydroxy-N-methylpyrrolidine-2-carboxamide and 2-chloro-4,6-bis(trifluoromethyl)pyridine as starting materials. The residue was purified by Prep-TLC (PE/EtOAc 1:1) to afford (2S,4S)-1-[4,6-bis(trifluoromethyl)pyridin-2-yl]-N-(4-fluorophenyl)-4-hydroxy-N-methylpyrrolidine-2-carboxamide (57.1 mg, 30.14%) as a white solid. ¹H NMR (300 MHz, DMSO-d₆): δ (ppm) 7.66-7.48 (m, 2H), 7.48-7.32 (m, 2H), 7.24 (s, 1H), 7.18-7.12 (m, 1H), 5.06-5.34 (m, 1H), 4.28-4.22 (m, 2H), 3.80-3.59 (m, 1H), 3.46-3.35 (m, 1H), 3.15-3.15 (d, 3H), 2.27-2.07 (m, 1H), 1.91-1.69 (m, 1H). m/z 452 (M+H⁺).

### Example 14

### Synthesis of (S)—N-(4-fluorophenyl)-N-methyl-1-(4-(trifluoromethyl)pyridin-2-yl)pyrrolidine-2-carboxamide

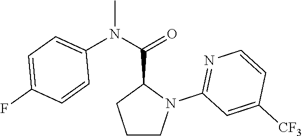 [SMILES: CN(C(=O)[C@@H]1CCCN1c1cc(C(F)(F)F)ccn1)c1ccc(F)cc1]

### Step 1: Preparation of (S)—N-(4-fluorophenyl)-N-methyl-1-(4-(trifluoromethyl)pyridin-2-yl)pyrrolidine-2-carboxamide

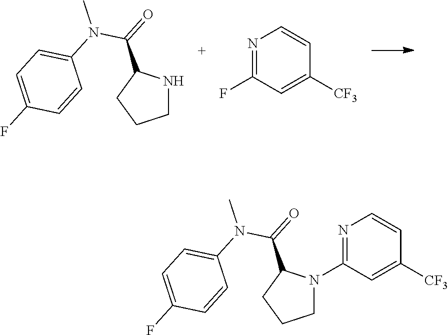

The title compound was prepared using General Procedure C, employing (2S)—N-(4-fluorophenyl)-N-methylpyrrolidine-2-carboxamide and 2-fluoro-4-(trifluoromethyl)pyridine as starting materials. The resulting mixture was concentrated under reduced pressure. The residue was purified by Prep-TLC (P/E 2:1 to afford (2S)—N-(4-fluorophenyl)-N-methyl-1-[4-(trifluoromethyl)pyridin-2-yl]pyrrolidine-2-carboxamide (22.2 mg, 13%) as a white solid. ¹H NMR (300 MHz, DMSO-d₆): δ (ppm) 8.32-8.31 (d, 1H), 7.59-7.49 (m, 2H), 7.43-7.34 (m, 2H), 6.81-6.79 (d, 1H), 6.61 (s, 1H), 4.36 (s, 1H), 3.62-3.42 (m, 2H), 3.12 (s, 3H) 2.28-1.80 (m, 4H). m/z 368 (M+H⁺).

### Example 15

### Synthesis of (S)—N-(4-fluorophenyl)-N-methyl-1-(6-(trifluoromethyl)pyridin-2-yl)pyrrolidine-2-carboxamide

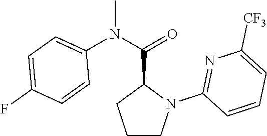 [SMILES: CN(C(=O)[C@@H]1CCCN1c1cccc(C(F)(F)F)n1)c1ccc(F)cc1]

### Step 1: Preparation of (S)—N-(4-fluorophenyl)-N-methyl-1-(6-(trifluoromethyl)pyridin-2-yl)pyrrolidine-2-carboxamide

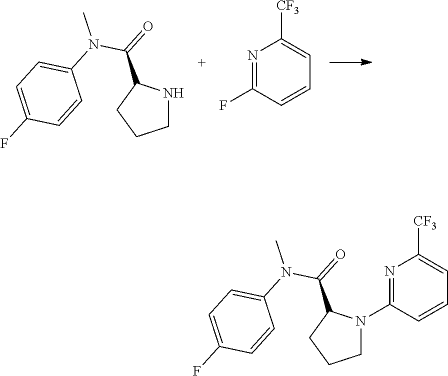

The title compound was prepared using General Procedure C, employing (2S)—N-(4-fluorophenyl)-N-methylpyrrolidine-2-carboxamide and 2-fluoro-6-(trifluoromethyl)pyridine as starting materials. The residue was purified by Prep-TLC (PE/EtOAc 1:1) to afford (2S)—N-(4-fluorophenyl)-N-methyl-1-[6-(trifluoromethyl)pyridin-2-yl]pyrrolidine-2-carboxamide (15.5 mg, 9%) as a colorless oil. ¹H NMR (300 MHz, CDCl₃): δ (ppm) 7.55-7.48 (m, 3H), 7.16-7.10 (m, 2H), 6.90-6.88 (d, 1H), 6.56-6.53 (d, 1H), 4.54-4.50 (m, 1H), 3.70-3.63 (m, 1H), 3.44-3.40 (m, 1H), 3.42 (s, 3H), 2.29-2.23 (m, 1H), 2.02-1.92 (m, 3H). m/z 368 (M+H⁺).

### Example 16

### Synthesis of (2S,4R)-1-(4,6-bis(trifluoromethyl)pyridin-2-yl)-N-(4-fluorophenyl)-4-hydroxy-N-methylpyrrolidine-2-carboxamide

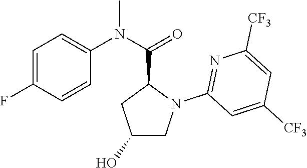 [SMILES: CN(C(=O)[C@@H]1C[C@@H](O)CN1c1cc(C(F)(F)F)cc(C(F)(F)F)n1)c1ccc(F)cc1]

### Step 1: Preparation of benzyl tert-butyl (2S,4R)-2-((4-fluorophenyl)(methyl)carbamoyl)-4-hydroxypyrrolidine-1-carboxylate

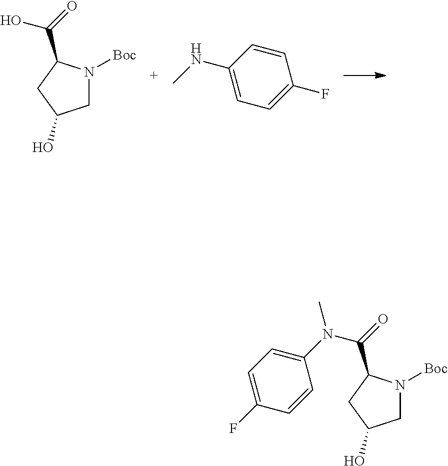

The title compound was prepared using General Procedure A, employing (2S,4R)-1-(tert-butoxycarbonyl)-4-hydroxypyrrolidine-2-carboxylic acid and 4-fluoro-N-methylaniline as starting materials. The residue was purified by silica gel column chromatography, eluted with CH₂Cl₂/MeOH (25:1) to afford tert-butyl (2R,4S)-2-[(4-fluorophenyl)(methyl)carbamoyl]-4-hydroxypyrrolidine-1-carboxylate as a light blue solid (300 mg, 21%).

### Step 2: Preparation of (2S,4R)—N-(4-fluorophenyl)-4-hydroxy-N-methylpyrrolidine-2-carboxamide

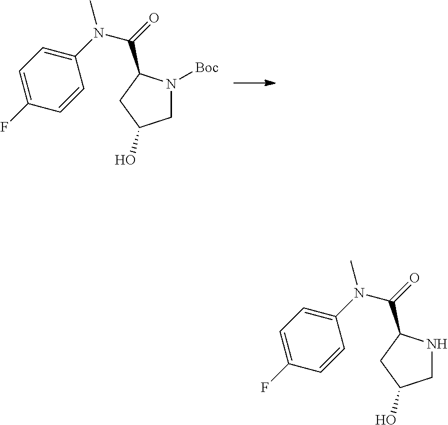 [SMILES: CN(C(=O)[C@@H]1C[C@@H](O)CN1C(=O)OC(C)(C)C)c1ccc(F)cc1 ; CN(C(=O)[C@@H]1C[C@@H](O)CN1)c1ccc(F)cc1]

The title compound was prepared using General Procedure B, tert-butyl (2S,4R)-2-[(4-fluorophenyl)(methyl)carbamoyl]-4-hydroxypyrrolidine-1-carboxylate as starting material. The resulting mixture was concentrated under reduced pressure to afford crude (2S,4R)—N-(4-fluorophenyl)-4-hydroxy-N-methylpyrrolidine-2-carboxamide.

### Step 3: Preparation of (2S,4R)-1-(4,6-bis(trifluoromethyl)pyridin-2-yl)-N-(4-fluorophenyl)-4-hydroxy-N-methylpyrrolidine-2-carboxamide

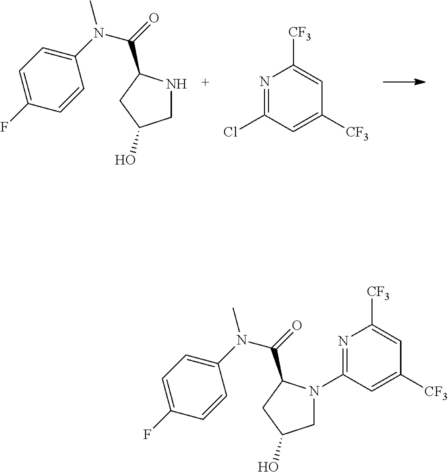 [SMILES: CN(C(=O)[C@@H]1C[C@@H](O)CN1)c1ccc(F)cc1 ; FC(F)(F)c1cc(Cl)nc(C(F)(F)F)c1 ; CN(C(=O)[C@@H]1C[C@@H](O)CN1c1cc(C(F)(F)F)cc(C(F)(F)F)n1)c1ccc(F)cc1]

The title compound was prepared using General Procedure C, employing (2S,4R)—N-(4-fluorophenyl)-4-hydroxy-N-methylpyrrolidine-2-carboxamide and 2-chloro-4,6-bis(trifluoromethyl)pyridine as starting materials. The residue was purified by Prep-TLC (CH₂Cl₂/MeOH 20:1) to afford (2S,4R)-1-(4,6-bis(trifluoromethyl)pyridin-2-yl)-N-(4-fluorophenyl)-4-hydroxy-N-methylpyrrolidine-2-carboxamide as a white solid. ¹H NMR (300 MHz, DMSO-d₆): δ (ppm) 7.52-7.48 (m, 2H), 7.38-7.32 (m, 2H), 7.23 (s, 1H), 7.11 (s, 1H), 5.04 (s, 1H), 4.42-4.38 (d, 2H), 3.68-3.63 (m, 1H), 3.46-3.42 (m, 1H), 3.13 (s, 3H), 2.07-2.04 (m, 1H), 1.91-1.85 (m, 1H). m/z 452 (M+H⁺).

### Example 17

### Synthesis of 1-(4,6-bis(trifluoromethyl)pyrimidin-2-yl)-N-(4-fluorophenyl)-N-methylpyrrolidine-2-carboxamide

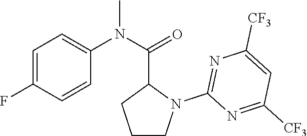 [SMILES: CN(C(=O)C1CCCN1c1nc(C(F)(F)F)cc(C(F)(F)F)n1)c1ccc(F)cc1]

### Step 1: Preparation of 4,6-bis(trifluoromethyl)pyrimidin-2-ol

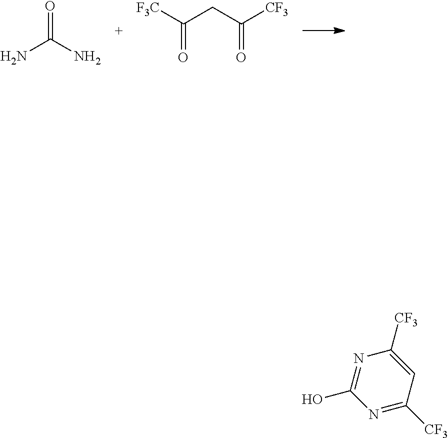

To a stirred solution hexafluoroacetylacetone (1.00 g, 4.806 mmol, 1.00 equiv) and urea (288.65 mg, 4.806 mmol, 1.00 equiv) in EtOH (15.00 mL) was added H₂SO₄ (0.15 mL, 2.814 mmol, 0.59 equiv). The resulting mixture was heated to 85° C. and stirred for 6 h. The reaction was quenched by the addition of aq. NaHCO₃ (30 mL) and EtOH was removed under reduced pressure. The remaining aqueous solution was extracted with diethyl ether (2×10 mL). The combined organic layers were washed with brine, dried over anhydrous sodium sulfate, filtered and concentrated under reduced pressure to afford 4,6-bis(trifluoromethyl)pyrimidin-2-ol (600 mg, 54%) as a yellow oil.

### Step 2: Preparation of 2-chloro-4,6-bis(trifluoromethyl)pyrimidine

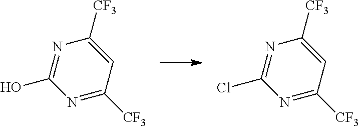 [SMILES: Oc1nc(C(F)(F)F)cc(C(F)(F)F)n1 ; FC(F)(F)c1cc(C(F)(F)F)nc(Cl)n1]

A solution of 4,6-bis(trifluoromethyl)pyrimidin-2-ol (600 mg, 2.59 mmol) in phosphorus oxychloride (3.00 mL, 32.2 mmol) was heated to 80° C. and stirred for 4 h. The reaction mixture was cooled to 0° C. and quenched by the addition of aq. NaHCO₃ (20 mL). The aqueous solution was extracted with diethyl ether (2×10 mL). The combined organic layers were washed with brine, dried over anhydrous sodium sulfate, filtered and concentrated under reduced pressure to afford 2-chloro-4,6-bis(trifluoromethyl)pyrimidine (100 mg, 15%) as a yellow oil.

### Step 3: Preparation of 1-(4,6-bis(trifluoromethyl)pyrimidin-2-yl)-N-(4-fluorophenyl)-N-methylpyrrolidine-2-carboxamide

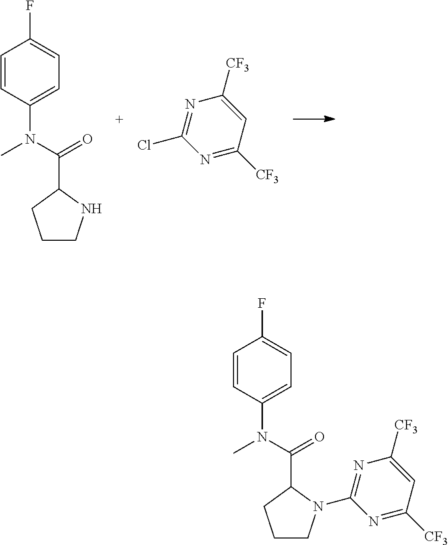 [SMILES: CN(C(=O)C1CCCN1)c1ccc(F)cc1 ; FC(F)(F)c1cc(C(F)(F)F)nc(Cl)n1 ; CN(C(=O)C1CCCN1c1nc(C(F)(F)F)cc(C(F)(F)F)n1)c1ccc(F)cc1]

The title compound was prepared using General Procedure C, employing N-(4-fluorophenyl)-N-methylpyrrolidine-2-carboxamide and 2-chloro-4,6-bis(trifluoromethyl)pyridine as starting materials. The residue was purified by silica gel column chromatography (eluent: 0-30% EtOAc in PE) to afford 1-[4,6-bis(trifluoromethyl)pyrimidin-2-yl]-N-(4-fluorophenyl)-N-methylpyrrolidine-2-carboxamide (25.5 mg, 15%) as a light yellow solid. ¹H NMR (300 MHz, CDCl₃): δ (ppm) 7.53-7.51 (m, 1H), 7.18-7.14 (m, 2H), 7.05 (s, 1H), 4.49-4.46 (m, 1H), 3.93-3.87 (m, 1H), 3.82-3.76 (m, 1H), 3.27 (s, 2H), 2.27-2.24 (m, 1H), 2.20-2.05 (m, 2H), 1.97-1.87 (m, 2H). m/z 437 (M+H⁺).

### Example 18

### Synthesis of (S)-1-(4,6-bis(trifluoromethyl)pyridin-2-yl)-N-(3-cyanophenyl)-N-methylpyrrolidine-2-carboxamide

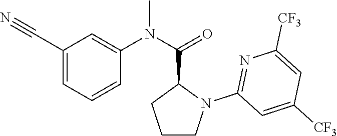 [SMILES: CN(C(=O)[C@@H]1CCCN1c1cc(C(F)(F)F)cc(C(F)(F)F)n1)c1cccc(C#N)c1]

### Step 1: Preparation of benzyl (S)-2-(chlorocarbonyl)pyrrolidine-1-carboxylate

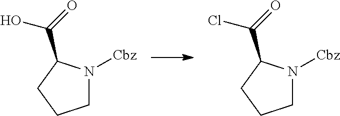 [SMILES: O=C(O)[C@@H]1CCCN1C(=O)OCc1ccccc1 ; O=C(Cl)[C@@H]1CCCN1C(=O)OCc1ccccc1]

To a solution of 1-[(benzyloxy)carbonyl]pyrrolidine-2-carboxylic acid (1.00 g, 4.012 mmol, 1.00 equiv) in DCM (10.00 mL) was added (COCl)₂ (0.61 g, 4.814 mmol, 1.20 equiv) followed by DMF (0.03 g, 0.401 mmol, 0.10 equiv). The resulting solution was stirred at 0° C. for 2 h and concentrated under reduced pressure to afford benzyl 2-(carboxy)pyrrolidine-1-carboxylate (1.00 g, 84%) as yellow oil.

### Step 2: Preparation of benzyl (S)-2-((3-cyanophenyl)(methyl)carbamoyl)pyrrolidine-1-carboxylate

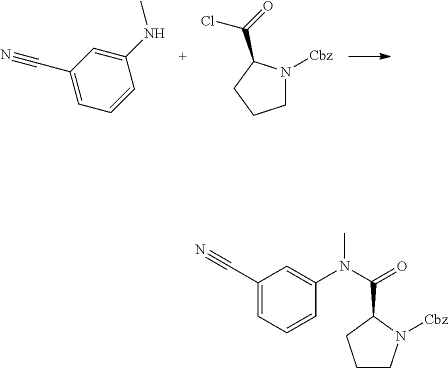

To a solution of 3-(methylamino)benzonitrile (197.48 mg, 1.494 mmol, 1.00 equiv) in DCM (4.00 mL) at room temperature was added Et₃N (302.39 mg, 2.988 mmol, 2.00 equiv) followed by benzyl 2-(carboxy)pyrrolidine-1-carboxylate (400.00 mg, 1.494 mmol, 1.00 equiv). The resulting solution was stirred at room temperature for 2 h. The reaction mixture was diluted with water (50 mL) and the solids were filtered off and washed with EtOAc (3×100 mL). The combined filtrate was concentrated under reduced pressure. The residue was purified by Prep-TLC (PE/EtOAc 10:1) to afford benzyl (2S)-2-[(3-cyanophenyl)(methyl)carbamoyl]pyrrolidine-1-carboxylate (300 mg, 54%) as a yellow solid.

### Step 3: Preparation of (S)—N-(3-cyanophenyl)-N-methylpyrrolidine-2-carboxamide

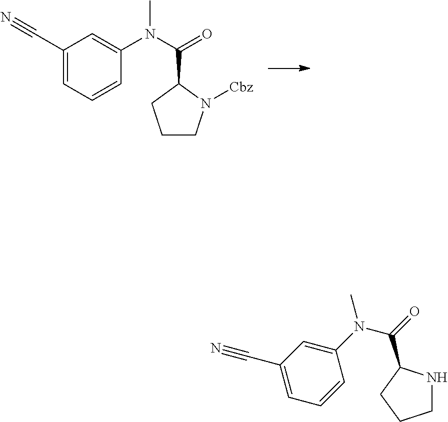 [SMILES: CN(C(=O)[C@@H]1CCCN1C(=O)OCc1ccccc1)c1cccc(C#N)c1 ; CN(C(=O)[C@@H]1CCCN1)c1cccc(C#N)c1]

The title compound was prepared using General Procedure B, employing benzyl (2S)-2-[(3-cyanophenyl)(methyl)carbamoyl]pyrrolidine-1-carboxylate as starting material. The residue was purified by Prep-TLC (PE/EtOAc 1:1) to afford (2S)—N-(3-cyanophenyl)-N-methylpyrrolidine-2-carboxamide (150 mg, 72%) as a yellow oil.

To a solution of benzyl (2S)-2-[(3-cyanophenyl)(methyl)carbamoyl]pyrrolidine-1-carboxylate (300.00 mg, 0.825 mmol, 1.00 equiv) in MeOH (10.00 mL) at room temperature was added Pd/C (30.00 mg). The resulting mixture was stirred at room temperature for 2.5 h under a balloon of hydrogen gas. The solids were filtered off and washed with MeOH (5 mL). The filtrate was concentrated and purified by Prep-TLC (PE/EtOAc 1:1) to afford (2S)—N-(3-cyanophenyl)-N-methylpyrrolidine-2-carboxamide (150 mg, 72%) as a yellow oil.

### Step 4: Preparation of (S)-1-(4,6-bis(trifluoromethyl)pyridin-2-yl)-N-(3-cyanophenyl)-N-methylpyrrolidine-2-carboxamide

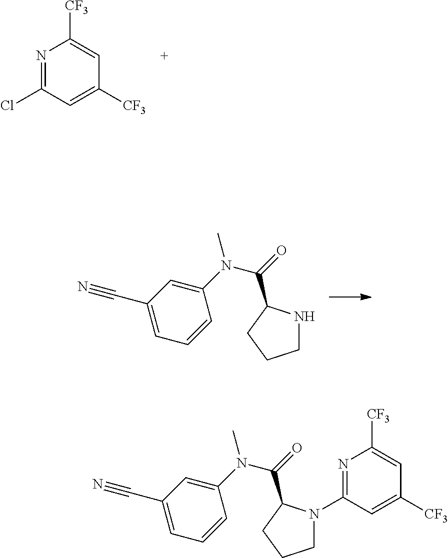 [SMILES: FC(F)(F)c1cc(Cl)nc(C(F)(F)F)c1 ; CN(C(=O)[C@@H]1CCCN1)c1cccc(C#N)c1 ; CN(C(=O)[C@@H]1CCCN1c1cc(C(F)(F)F)cc(C(F)(F)F)n1)c1cccc(C#N)c1]

The title compound was prepared using General Procedure C, employing (2S)—N-(3-cyanophenyl)-N-methylpyrrolidine-2-carboxamide and 2-chloro-4,6-bis(trifluoromethyl)pyridine as starting materials. The residue was purified by Prep-TLC (PE/EtOAc 1:1) to afford (S)-1-(4,6-bis(trifluoromethyl)pyridin-2-yl)-N-(3-cyanophenyl)-N-methylpyrrolidine-2-carboxamide (45.7 mg, 16%) as a white solid. ¹H NMR (300 MHz, DMSO-d₆): δ (ppm) 8.30-7.51 (m, 4H), 7.23 (s, 1H), 7.08 (s, 1H), 4.34 (s, 1H), 3.65-3.53 (m, 2H), 3.14 (s, 3H), 2.13-1.91 (m, 4H). m/z 443 (M+H⁺).

### Example 19

### Synthesis of (2S,3S)-1-(4,6-bis(trifluoromethyl)pyridin-2-yl)-N-(4-fluorophenyl)-3-hydroxy-N-methylpyrrolidine-2-carboxamide

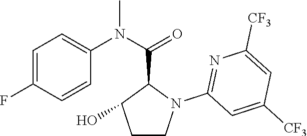 [SMILES: CN(C(=O)[C@@H]1[C@@H](O)CCN1c1cc(C(F)(F)F)cc(C(F)(F)F)n1)c1ccc(F)cc1]

### Step 1: Preparation of tert-butyl (2S,3S)-2-((4-fluorophenyl)(methyl)carbamoyl)-3-hydroxypyrrolidine-1-carboxylate

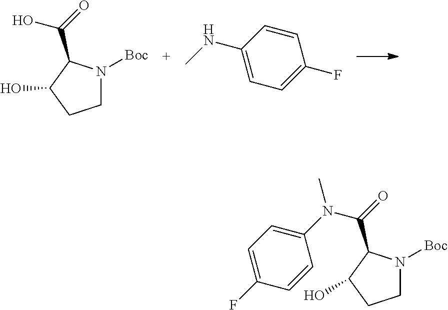

To a solution of (2S,3S)-1-(tert-butoxycarbonyl)-3-hydroxypyrrolidine-2-carboxylic acid (500.00 mg, 2.162 mmol, 1.00 equiv) in pyridine (16.70 mL) at 0° C. was added HATU (1233.19 mg, 3.243 mmol, 1.50 equiv). The resulting solution was stirred for 15 min and 4-fluoro-N-methylaniline (541.18 mg, 4.324 mmol, 2.00 equiv) was added. The reaction mixture was heated to 70° C. and stirred for an additional 3 h. The mixture was concentrated under reduced pressure and the residue was purified by Prep-TLC (CH₂Cl₂/MeOH 15:1) to afford tert-butyl (2S,3S)-2-[(4-fluorophenyl)(methyl)carbamoyl]-3-hydroxypyrrolidine-1-carboxylate (300 mg, 35%) as a yellow solid.

### Step 2: Preparation of (2S,3S)—N-(4-fluorophenyl)-3-hydroxy-N-methylpyrrolidine-2-carboxamide

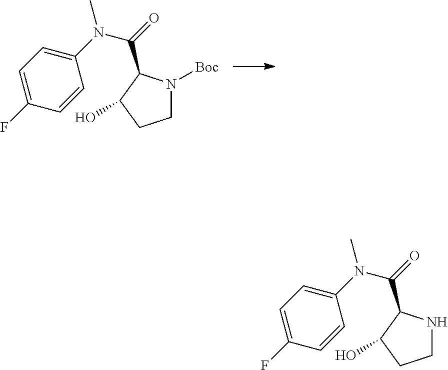 [SMILES: CN(C(=O)[C@@H]1[C@@H](O)CCN1C(=O)OC(C)(C)C)c1ccc(F)cc1 ; CN(C(=O)[C@H]1NCC[C@@H]1O)c1ccc(F)cc1]

The title compound was prepared using General Procedure B, employing tert-butyl (2S,3S)-2-[(4-fluorophenyl)(methyl)carbamoyl]-3-hydroxypyrrolidine-1-carboxylate as starting material. The solution was concentrated under reduced pressure to afford (2S,3S)—N-(4-fluorophenyl)-3-hydroxy-N-methylpyrrolidine-2-carboxamide (200 mg, 88%) as yellow oil.

### Step 3: Preparation of (2S,3S)-1-(4,6-bis(trifluoromethyl)pyridine-2-yl)-N-(4-fluorophenyl)-3-hydroxy-N-methylpyrrolidine-2-carboxamide

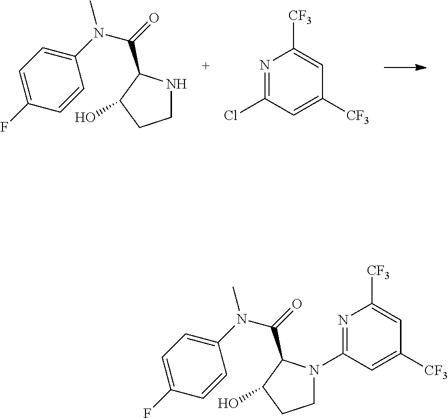 [SMILES: CN(C(=O)[C@H]1NCC[C@@H]1O)c1ccc(F)cc1 ; FC(F)(F)c1cc(Cl)nc(C(F)(F)F)c1 ; CN(C(=O)[C@@H]1[C@@H](O)CCN1c1cc(C(F)(F)F)cc(C(F)(F)F)n1)c1ccc(F)cc1]

The title compound was prepared using General Procedure C, employing (2S,3S)—N-(4-fluorophenyl)-3-hydroxy-N-methylpyrrolidine-2-carboxamide and 2-chloro-4,6-bis(trifluoromethyl)pyridine as starting materials. The residue was purified by Prep-TLC (CH₂Cl₂/MeOH 40:1) to afford (2S,3S)-1-[4,6-bis(trifluoromethyl)pyridin-2-yl]-N-(4-fluorophenyl)-3-hydroxy-N-methylpyrrolidine-2-carboxamide (58.2 mg, 20%) as a white solid. ¹H NMR (300 MHz, DMSO-d₆): δ (ppm) 7.57-7.53 (d, 2H), 7.41-7.30 (t, 2H), 7.24-7.09 (m, 2H), 5.09 (s, 1H), 4.34 (s, 2H), 3.71-3.56 (d, 2H), 3.12 (s, 3H), 2.27-2.14 (m, 1H), 1.98-1.89 (m, 1H). m/z 452 (M+H⁺).

### Example 20

### Synthesis of (2S,4S)-4-acetamido-1-(4,6-bis(trifluoromethyl)pyridin-2-yl)-N-(4-fluorophenyl)-N-methylpyrrolidine-2-carboxamide

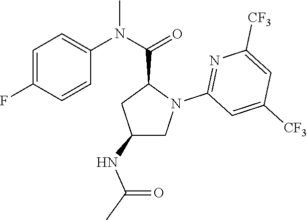 [SMILES: CC(=O)N[C@H]1C[C@@H](C(=O)N(C)c2ccc(F)cc2)N(c2cc(C(F)(F)F)cc(C(F)(F)F)n2)C1]

### Step 1: Preparation of (2S,4S)-4-acetamido-1-(tert-butoxycarbonyl)pyrrolidine-2-carboxylic acid

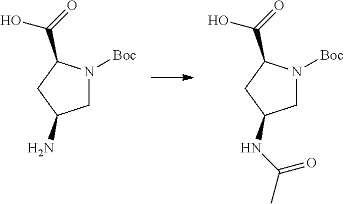 [SMILES: CC(C)(C)OC(=O)N1C[C@@H](N)C[C@H]1C(=O)O ; CC(=O)N[C@H]1C[C@@H](C(=O)O)N(C(=O)OC(C)(C)C)C1]

To a solution of (2S,4S)-4-amino-1-(tert-butoxycarbonyl)pyrrolidine-2-carboxylic acid (550.00 mg, 2.389 mmol, 1.00 equiv) in DCM (5 mL) at room temperature was added (Ac)₂O (292.61 mg, 2.866 mmol, 1.20 equiv) and Et₃N (725.10 mg, 7.166 mmol, 3 equiv). The resulting mixture was stirred at room temperature for 4 h. The reaction mixture was diluted with water (20 mL) and the aqueous layer was extracted with EtOAc (3×10 mL). The combined organic extracts were dried over anhydrous sodium sulfate, filtered and concentrated under reduced pressure. The residue was purified by reverse-phase flash chromatography (conditions: column—C18 silica gel; mobile phase—MeCN in water (0.05% FA), 0% to 100% gradient over 60 min, UV 220 nm). The pure fractions were combined together and lyophilized to afford (2S,4S)-4-acetamido-1-(tert-butoxycarbonyl)pyrrolidine-2-carboxylic acid (590 mg, 87%) as a white solid.

### Step 2: Preparation of tert-butyl (2S,4S)-4-acetamido-2-((4-fluorophenyl)(methyl)carbamoyl)pyrrolidine-1-carboxylate

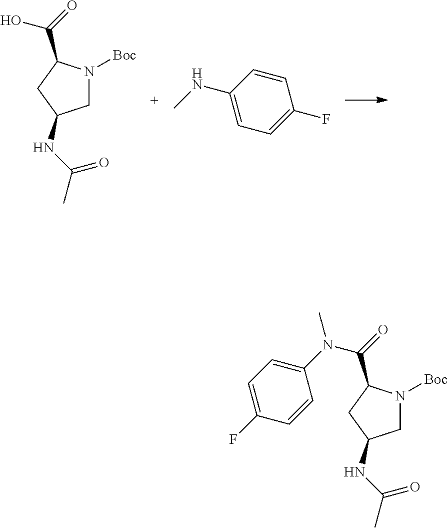

The title compound was prepared using General Procedure A, employing (2S,4S)-1-(tert-butoxycarbonyl)-4-acetamidopyrrolidine-2-carboxylic acid and 4-fluoro-N-methylaniline as starting materials. The residue was purified by reverse-phase flash chromatography (conditions: column—C₁₈ silica gel; mobile phase—MeCN in water (0.05% FA), 0% to 100% gradient over 60 min, UV 220 nm). The pure fractions were combined together and lyophilized to afford tert-butyl (2S,4S)-4-acetamido-2-[(4-fluorophenyl)(methyl)carbamoyl]pyrrolidine-1-carboxylate (180 mg, 28%) as a white solid.

### Step 3: Preparation of (2S,4S)-4-acetamido-N-(4-fluorophenyl)-N-methylpyrrolidine-2-carboxamide

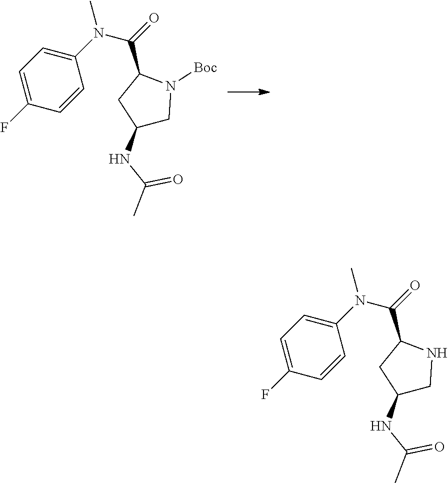 [SMILES: CC(=O)N[C@H]1C[C@@H](C(=O)N(C)c2ccc(F)cc2)N(C(=O)OC(C)(C)C)C1 ; CC(=O)N[C@@H]1CN[C@H](C(=O)N(C)c2ccc(F)cc2)C1]

The title compound was prepared using General Procedure B, (2S,4S)-tert-butyl 4-acetamido-2-((4-fluorophenyl)(methyl)carbamoyl)pyrrolidine-1-carboxylate as starting material. The reaction mixture was concentrated under reduced pressure to afford (2S,4S)-4-acetamido-N-(4-fluorophenyl)-N-methylpyrrolidine-2-carboxamide (110 mg, 98%) as a white solid

### Step 4: Preparation of (2S,4S)-4-acetamido-1-(4,6-bis(trifluoromethyl)pyridin-2-yl)-N-(4-fluorophenyl)-N-methylpyrrolidine-2-carboxamide

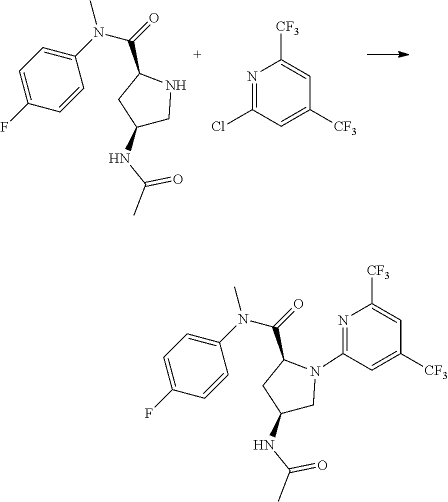 [SMILES: CC(=O)N[C@@H]1CN[C@H](C(=O)N(C)c2ccc(F)cc2)C1 ; FC(F)(F)c1cc(Cl)nc(C(F)(F)F)c1 ; CC(=O)N[C@H]1C[C@@H](C(=O)N(C)c2ccc(F)cc2)N(c2cc(C(F)(F)F)cc(C(F)(F)F)n2)C1]

The title compound was prepared using General Procedure C, employing (2S,4S)-4-acetamido-N-(4-fluorophenyl)-N-methylpyrrolidine-2-carboxamide and 2-chloro-4,6-bis(trifluoromethyl)pyridine as starting materials. The residue was purified by Prep-TLC (PE/EtOAc=5:1) to afford (2S,4S)-4-acetamido-1-(4,6-bis(trifluoromethyl)pyridin-2-yl)-N-(4-fluorophenyl)-N-methylpyrrolidine-2-carboxamide (38.4 mg, 20%) as a white solid. ¹H NMR (300 MHz, DMSO): δ (ppm) 8.15-8.13 (m, 1H), 7.51-7.46 (m, 2H), 7.39-7.36 (t, 2H), 7.27 (s, 1H), 7.21-7.14 (m, 1H), 4.37-4.31 (t, 2H), 3.92-3.87 (m, 1H), 3.33-3.28 (m, 1H), 3.13 (s, 3H), 2.28-2.16 (m, 1H), 1.91-1.85 (m, 4H). m/z 493 (M+H⁺).

### Example 21

### Synthesis of (2S,5S)-1-(4,6-bis(trifluoromethyl)pyridin-2-yl)-N-(4-fluorophenyl)-N,5-dimethylpyrrolidine-2-carboxamide

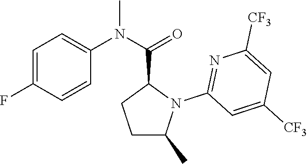 [SMILES: C[C@H]1CC[C@@H](C(=O)N(C)c2ccc(F)cc2)N1c1cc(C(F)(F)F)cc(C(F)(F)F)n1]

### Step 1: Preparation of tert-butyl (2S,5S)-2-((4-fluorophenyl)(methyl)carbamoyl)-5-methylpyrrolidine-1-carboxylate

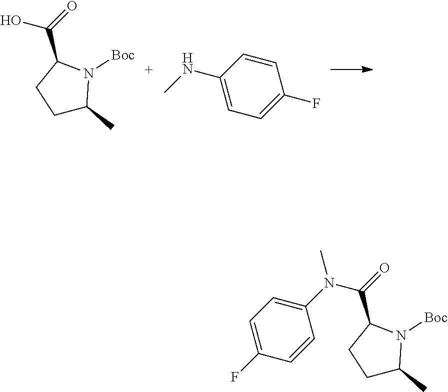

The title compound was prepared using General Procedure A, employing (2S,5S)-1-(tert-butoxycarbonyl)-5-methylpyrrolidine-2-carboxylic acid and 4-fluoro-N-methylaniline as starting materials. The residue was purified by silica gel column chromatography (eluent: 0-10% EtOAc in PE) to afford (2S,5S)-tert-butyl 2-((4-fluorophenyl)(methyl)carbamoyl)-5-methylpyrrolidine-1-carboxylate (920 mg, 61% yield) as a grey oil.

### Step 2: Preparation of (2S,5S)—N-(4-fluorophenyl)-N,5-dimethylpyrrolidine-2-carboxamide

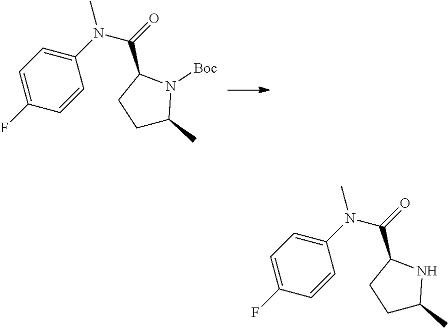 [SMILES: C[C@H]1CC[C@@H](C(=O)N(C)c2ccc(F)cc2)N1C(=O)OC(C)(C)C ; C[C@H]1CC[C@@H](C(=O)N(C)c2ccc(F)cc2)N1]

The title compound was prepared using General Procedure B, employing (2S,5S)-tert-butyl 2-((4-fluorophenyl)(methyl)carbamoyl)-5-methylpyrrolidine-1-carboxylate as starting material. The reaction mixture was concentrated under reduced pressure to afford (2S,5S)—N-(4-fluorophenyl)-N,5-dimethylpyrrolidine-2-carboxamide (550 mg, crude) as a grey oil.

### Step 3: Preparation of (2S,5S)-1-(4,6-bis(trifluoromethyl)pyridin-2-yl)-N-(4-fluorophenyl)-N,5-dimethylpyrrolidine-2-carboxamide

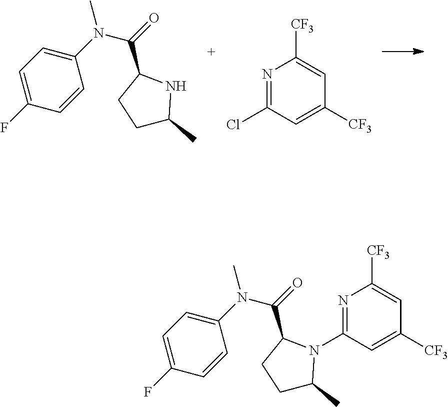

The title compound was prepared using General Procedure C, employing (2S,5S)—N-(4-fluorophenyl)-N,5-dimethylpyrrolidine-2-carboxamide and 2-chloro-4,6-bis(trifluoromethyl)pyridine as starting materials. The residue was purified by Prep-TLC (hexane/EtOAc 3:1) to afford (2S,5S)-1-(4,6-bis(trifluoromethyl)pyridin-2-yl)-N-(4-fluorophenyl)-N,5-dimethylpyrrolidine-2-carboxamide (74 mg, 100% yield) as a white solid. ¹H NMR (300 MHz, DMSO-d₆) δ (ppm) 7.51-7.47 (m, 2H), 7.37-7.31 (t, 2H), 7.19 (s, 1H), 7.00 (s, 1H), 4.27-4.22 (m, 2H), 3.13 (s, 3H), 2.07-1.93 (m, 3H), 1.75-1.64 (m, 1H), 1.25-1.23 (d, 3H). m/z 450 [M+H]+.

### Example 22

### Synthesis of (2S,4R)-4-acetamido-1-(4,6-bis(trifluoromethyl)pyridin-2-yl)-N-(4-fluorophenyl)-N-methylpyrrolidine-2-carboxamide

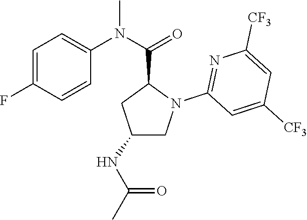 [SMILES: CC(=O)N[C@@H]1C[C@@H](C(=O)N(C)c2ccc(F)cc2)N(c2cc(C(F)(F)F)cc(C(F)(F)F)n2)C1]

### Step 1: Preparation of (2S,4R)-4-acetamido-1-(tert-butoxycarbonyl)pyrrolidine-2-carboxylic acid

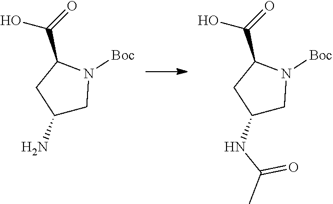 [SMILES: CC(C)(C)OC(=O)N1C[C@H](N)C[C@H]1C(=O)O ; CC(=O)N[C@@H]1C[C@@H](C(=O)O)N(C(=O)OC(C)(C)C)C1]

To a stirred solution of (2S,4R)-4-amino-1-(tert-butoxycarbonyl)pyrrolidine-2-carboxylic acid (500.00 mg, 2.171 mmol, 1.00 equiv) and DIEA (561.28 mg, 4.343 mmol, 2 equiv) in DCM (5.00 mL) at room temperature was added acetyl chloride (204.55 mg, 2.606 mmol, 1.20 equiv). The resulting mixture was stirred at room temperature for 3 h. The reaction was quenched by adding H₂O (10 mL) and the resulting mixture was extracted with EtOAc (2×30 mL). The combined organic extracts were dried over anhydrous sodium sulfate, filtered and concentrated under reduced pressure. The residue was purified by reverse-phase flash chromatography (conditions: column—C18 silica gel; mobile phase -MeCN in water, 0% to 38% gradient over 17 min; detector—UV 220 nm) to afford (2S,4R)-1-(tert-butoxycarbonyl)-4-acetamidopyrrolidine-2-carboxylic acid (390 mg, 64%) as a colorless liquid.

### Step 2: Preparation of tert-butyl (2S,4R)-4-acetamido-2-((4-fluorophenyl)(methyl)carbamoyl)pyrrolidine-1-carboxylate

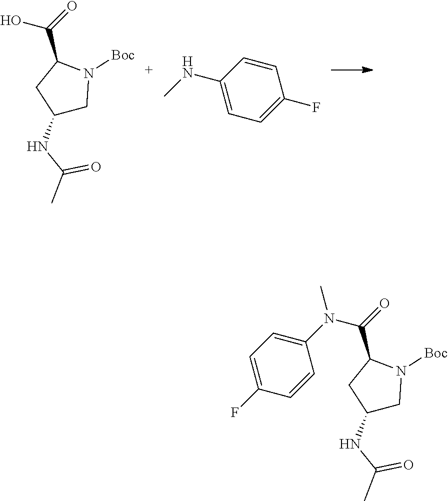

The title compound was prepared using General Procedure A, employing (2S,4R)-1-(tert-butoxycarbonyl)-4-acetamidopyrrolidine-2-carboxylic acid and 4-fluoro-N-methylaniline as starting materials. The residue was purified by reverse-phase flash chromatography (conditions: column—C18 silica gel; mobile phase—MeCN in water, 0% to 50% gradient over 27 min; detector—UV 220 nm) to afford tert-butyl (2S,4R)-4-acetamido-2-[(4-fluorophenyl)(methyl)carbamoyl]pyrrolidine-1-carboxylate (80 mg, 14.05%) as a colorless oil.

### Step 3: Preparation of (2S,4R)-4-acetamido-N-(4-fluorophenyl)-N-methylpyrrolidine-2-carboxamide

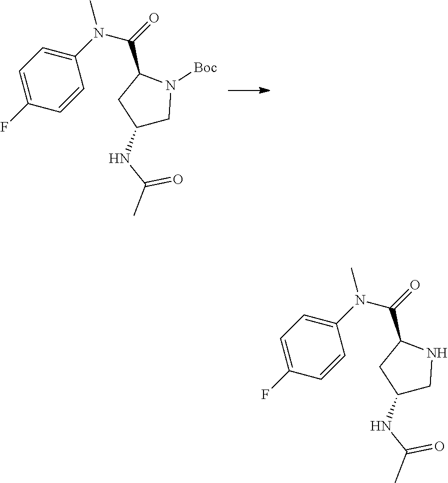 [SMILES: CC(=O)N[C@@H]1C[C@@H](C(=O)N(C)c2ccc(F)cc2)N(C(=O)OC(C)(C)C)C1 ; CC(=O)N[C@H]1CN[C@H](C(=O)N(C)c2ccc(F)cc2)C1]

The title compound was prepared using General Procedure B, employing tert-butyl (2S,4R)-4-acetamido-2-[(4-fluorophenyl)(methyl)carbamoyl]pyrrolidine-1-carboxylate as starting material. The crude product was used directly in the next step without further purification.

### Step 4: Preparation of (2S,4R)-4-acetamido-1-(4,6-bis(trifluoromethyl)pyridin-2-yl)-N-(4-fluorophenyl)-N-methylpyrrolidine-2-carboxamide

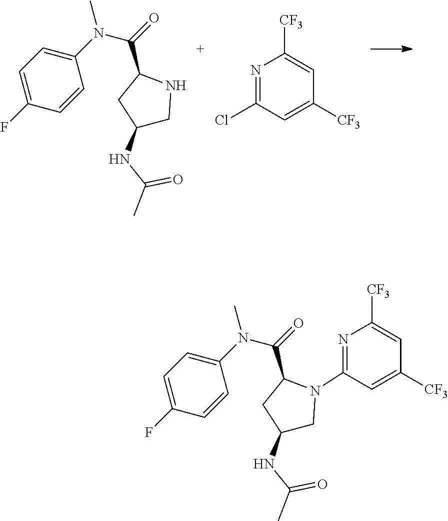 [SMILES: CC(=O)N[C@@H]1CN[C@H](C(=O)N(C)c2ccc(F)cc2)C1 ; FC(F)(F)c1cc(Cl)nc(C(F)(F)F)c1 ; CC(=O)N[C@H]1C[C@@H](C(=O)N(C)c2ccc(F)cc2)N(c2cc(C(F)(F)F)cc(C(F)(F)F)n2)C1]

The title compound was prepared using General Procedure C, employing (3R)-5-acetamido-N-(4-fluorophenyl)-N-methylpyrrolidine-3-carboxamide and 2-chloro-4,6-bis(trifluoromethyl)pyridine as starting materials. The residue was purified by Prep-TLC (hexane/EtOAc 5:1) to afford (2S,4R)-1-[4,6-bis(trifluoromethyl)pyridin-2-yl]-4-acetamido-N-(4-fluorophenyl)-N-methylpyrrolidine-2-carboxamide (26.6 mg, 20%) as a white solid. ¹H NMR (300 MHz, DMSO-d₆): δ (ppm) 8.08-8.06 (d, 1H), 7.52-7.48 (m, 2H), 7.39-7.33 (t, 1H), 7.27 (s, 1H), 7.26-7.13 (m, 1H), 4.51-4.44 (m, 2H), 3.81-3.76 (m, 1H), 3.40-3.46 (m, 1H), 3.11 (s, 3H), 2.22-2.19 (m, 1H), 1.92-1.82 (m, 1H), 1.74 (s, 3H). m/z 493 (M+H⁺).

### Example 23

### Synthesis of (2S,5R)-1-(4,6-bis(trifluoromethyl)pyridin-2-yl)-N-(4-fluorophenyl)-N,5-dimethylpyrrolidine-2-carboxamide

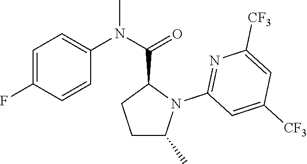 [SMILES: C[C@@H]1CC[C@@H](C(=O)N(C)c2ccc(F)cc2)N1c1cc(C(F)(F)F)cc(C(F)(F)F)n1]

### Step 1: Preparation of tert-butyl (2S,5R)-2-((4-fluorophenyl)(methyl)carbamoyl)-5-methylpyrrolidine-1-carboxylate

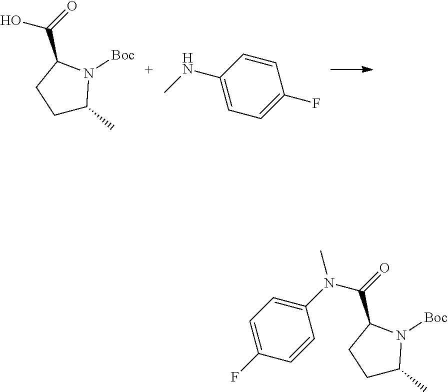

The title compound was prepared using General Procedure A, employing (2S,5R)-1-(tert-butoxycarbonyl)-5-methylpyrrolidine-2-carboxylic acid and 4-fluoro-N-methylaniline as starting materials. The residue was purified by Prep-TLC (CH₂Cl₂/MeOH 40:1) to afford tert-butyl (2S,4S)-2-[(4-fluorophenyl)(methyl)carbamoyl]-4-hydroxypyrrolidine-1-carboxylate (120 mg, 8%) as a brown oil. The residue was purified by silica gel column chromatography (eluent: 0-10% EtOAc in PE) to afford (2S,5R)-tert-butyl 2-((4-fluorophenyl)(methyl)carbamoyl)-5-methylpyrrolidine-1-carboxylate (450 mg, 29% yield) as a grey oil.

### Step 2: Preparation of (2S,5R)—N-(4-fluorophenyl)-N,5-dimethylpyrrolidine-2-carboxamide

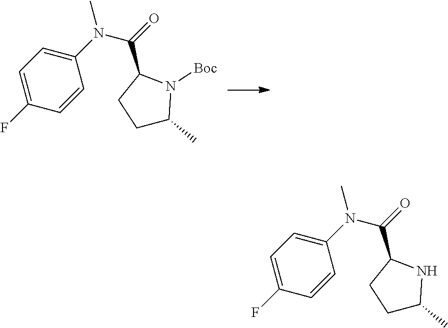 [SMILES: C[C@@H]1CC[C@@H](C(=O)N(C)c2ccc(F)cc2)N1C(=O)OC(C)(C)C ; C[C@@H]1CC[C@@H](C(=O)N(C)c2ccc(F)cc2)N1]

The title compound was prepared using General Procedure B, employing (2S,5R)-tert-butyl 2-((4-fluorophenyl)(methyl)carbamoyl)-5-methylpyrrolidine-1-carboxylate as starting material. The crude product was used directly in the next step without further purification.

### Step 3: Preparation of (2S,5R)-1-(4,6-bis(trifluoromethyl)pyridin-2-yl)-N-(4-fluorophenyl)-N,5-dimethylpyrrolidine-2-carboxamide

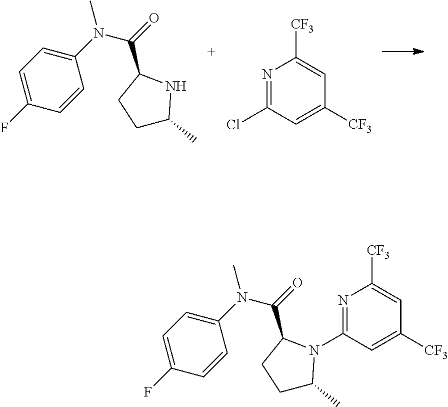

The title compound was prepared using General Procedure C, employing (2S,5R)—N-(4-fluorophenyl)-N,5-dimethylpyrrolidine-2-carboxamide and 2-chloro-4,6-bis(trifluoromethyl)pyridine as starting materials. The residue was purified by Prep-TLC (hexane/EtOAc 3:1) to afford (2S,5R)-1-(4,6-bis(trifluoromethyl)pyridin-2-yl)-N-(4-fluorophenyl)-N,5-dimethylpyrrolidine-2-carboxamide (20.3 mg, 5.3%) as a white solid. ¹H NMR (300 MHz, DMSO-d₆): δ (ppm) 7.51-7.47 (m, 2H), 7.37-7.31 (t, 2H), 7.19 (s, 1H), 7.00 (s, 1H), 4.24-4.22 (d, 2H), 3.13 (s, 3H), 2.07-1.91 (m, 3H), 1.70-1.68 (m, 1H), 1.25-1.23 (d, 3H). m/z 450 [M+H]⁺.

### Example 24

### Synthesis of 1-(3-cyano-4,6-bis(trifluoromethyl)pyridin-2-yl)-N-(4-fluorophenyl)-N-methylpyrrolidine-2-carboxamide

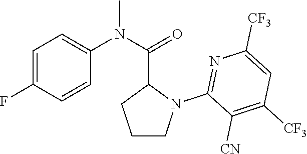 [SMILES: CN(C(=O)C1CCCN1c1nc(C(F)(F)F)cc(C(F)(F)F)c1C#N)c1ccc(F)cc1]

### Step 1: Preparation of 1-(3-cyano-4,6-bis(trifluoromethyl)pyridin-2-yl)-N-(4-fluorophenyl)-N-methylpyrrolidine-2-carboxamide

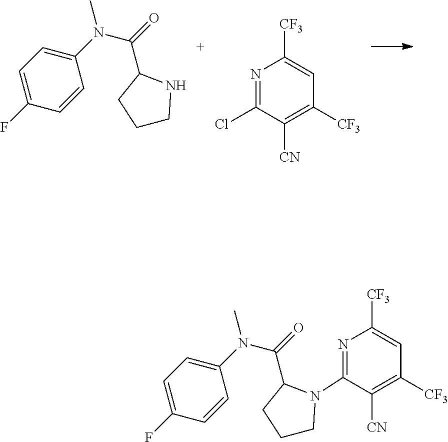 [SMILES: CN(C(=O)C1CCCN1)c1ccc(F)cc1 ; N#Cc1c(C(F)(F)F)cc(C(F)(F)F)nc1Cl ; CN(C(=O)C1CCCN1c1nc(C(F)(F)F)cc(C(F)(F)F)c1C#N)c1ccc(F)cc1]

The title compound was prepared using General Procedure C, employing N-(4-fluorophenyl)-N-methylpyrrolidine-2-carboxamide and 2-chloro-4,6-bis(trifluoromethyl)nicotinonitrile as starting materials. The residue was purified by prep-HPLC. The pure fractions were combined and lyophilized to afford 1-(3-cyano-4,6-bis(trifluoromethyl)pyridin-2-yl)-N-(4-fluorophenyl)-N-methylpyrrolidine-2-carboxamide (8 mg, 7.8%) as a white solid. ¹H NMR (400 MHz, Chloroform-d) δ (ppm) 7.38 (t, J=6.3 Hz, 2H), 7.20-7.09 (m, 3H), 4.61 (t, J=5.9 Hz, 1H), 4.20-4.03 (m, 2H), 3.22 (d, J=1.9 Hz, 3H), 1.96 (p, J=8.2, 7.6 Hz, 4H). m/z 461 [M+H]⁺.

### Example 25

### Synthesis of (S)-1-(3-cyano-4,6-bis(trifluoromethyl)pyridin-2-yl)-N-(4-fluorophenyl)-N-methylazetidine-2-carboxamide

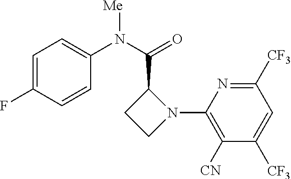 [SMILES: CN(C(=O)[C@@H]1CCN1c1nc(C(F)(F)F)cc(C(F)(F)F)c1C#N)c1ccc(F)cc1]

### Step 1: Preparation of tert-butyl (S)-2-((4-fluorophenyl)(methyl)carbamoyl)azetidine-1-carboxylate

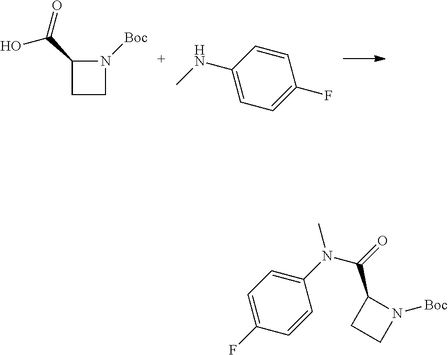

The title compound was prepared using General Procedure A, employing (S)-1-(tert-butoxycarbonyl)azetidine-2-carboxylic acid and 4-fluoro-N-methylaniline as starting materials. The residue was purified by silica gel column chromatography (eluent: 0-20% EtOAc in PE) to afford tert-butyl (S)-2-((4-fluorophenyl)(methyl)carbamoyl)azetidine-1-carboxylate as yellow oil (400 mg, 87%).

### Step 2: Preparation of (S)—N-(4-fluorophenyl)-N-methylazetidine-2-carboxamide

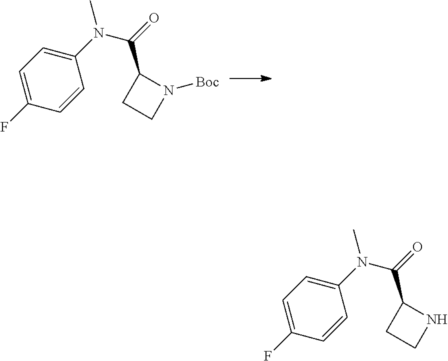 [SMILES: CN(C(=O)[C@@H]1CCN1C(=O)OC(C)(C)C)c1ccc(F)cc1 ; CN(C(=O)[C@@H]1CCN1)c1ccc(F)cc1]

The title compound was prepared using General Procedure B, employing tert-butyl (S)-2-((4-fluorophenyl)(methyl)carbamoyl)azetidine-1-carboxylate as starting material. The crude product was used directly in the next step without further purification.

### Step 3: Preparation of (S)-1-(3-cyano-4,6-bis(trifluoromethyl)pyridin-2-yl)-N-(4-fluorophenyl)-N-methylazetidine-2-carboxamide

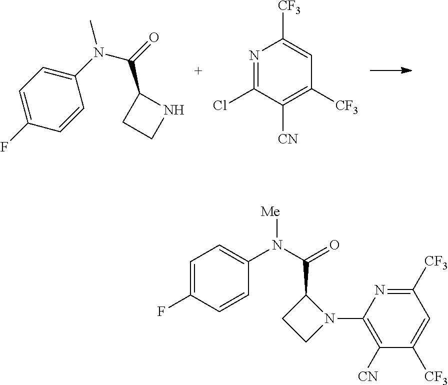 [SMILES: CN(C(=O)[C@@H]1CCN1)c1ccc(F)cc1 ; N#Cc1c(C(F)(F)F)cc(C(F)(F)F)nc1Cl ; CN(C(=O)[C@@H]1CCN1c1nc(C(F)(F)F)cc(C(F)(F)F)c1C#N)c1ccc(F)cc1]

The title compound was prepared using General Procedure C, employing (S)—N-(4-fluorophenyl)-N-methylazetidine-2-carboxamide and 2-chloro-4,6-bis(trifluoromethyl)nicotinonitrile as starting materials. The residue was purified by prep-HPLC. The pure fractions were combined and lyophilized to afford (S)-1-(3-cyano-4,6-bis(trifluoromethyl)pyridin-2-yl)-N-(4-fluorophenyl)-N-methylazetidine-2-carboxamide (5 mg, 1.2%) as a white solid. ¹H NMR (400 MHz, DMSO-d₆) δ (ppm) 7.46 (s, 1H), 7.41-7.28 (m, 4H), 4.25 (s, 1H), 3.46-3.24 (m, 1H), 3.18 (s, 3H), 2.41-2.20 (m, 2H), 1.56-1.05 (m, 1H); m/z 447 [M+H]⁺.

### Example 26 and 27

### Synthesis of (S)-2-(2-(3-(4-fluorophenyl)pyridin-2-yl)pyrrolidin-1-yl)-4,6-bis(trifluoromethyl)pyridine and (R)-2-(2-(3-(4-fluorophenyl)pyridin-2-yl)pyrrolidin-1-yl)-4,6-bis(trifluoromethyl)pyridine

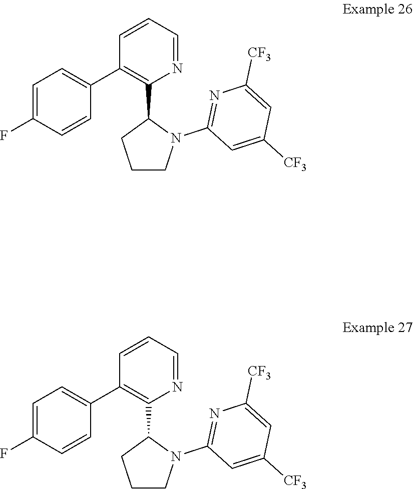 [SMILES: Fc1ccc(-c2cccnc2[C@@H]2CCCN2c2cc(C(F)(F)F)cc(C(F)(F)F)n2)cc1 ; Fc1ccc(-c2cccnc2[C@H]2CCCN2c2cc(C(F)(F)F)cc(C(F)(F)F)n2)cc1]

### Step 1: Preparation of(S)-2-(2-(3-(4-fluorophenyl)pyridin-2-yl)pyrrolidin-1-yl)-4,6-bis(trifluoromethyl)pyridine and (R)-2-(2-(3-(4-fluorophenyl)pyridin-2-yl)pyrrolidin-1-yl)-4,6-bis(trifluoromethyl)pyridine

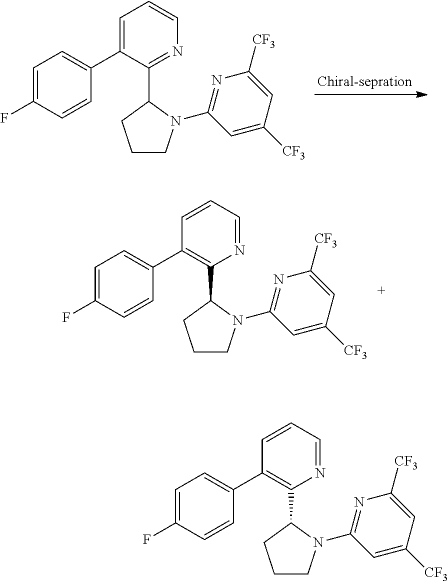 [SMILES: Fc1ccc(-c2cccnc2C2CCCN2c2cc(C(F)(F)F)cc(C(F)(F)F)n2)cc1 ; Fc1ccc(-c2cccnc2[C@@H]2CCCN2c2cc(C(F)(F)F)cc(C(F)(F)F)n2)cc1 ; Fc1ccc(-c2cccnc2[C@H]2CCCN2c2cc(C(F)(F)F)cc(C(F)(F)F)n2)cc1]

The crude product 2-[2-(3-phenylpyridin-2-yl)pyrrolidin-1-yl]-4,6-bis(trifluoromethyl)pyridine (110.00 mg) was purified by Chiral-Prep-HPLC using the following conditions: Column—UniChiral OD-5H, 30*250 mm, 5 um; mobile phase—Hex (8 mmol/L NH₃·MeOH) and IPA (hold 1% IPA for 6 min); detector—UV 254/220. The pure fractions were combined and lyophilized to afford 2-[(2S)-2-[3-(4-fluorophenyl)pyridin-2-yl]pyrrolidin-1-yl]-4,6-bis(trifluoromethyl)pyridine (24.8 mg) and 2-[(2R)-2-[3-(4-fluorophenyl)pyridin-2-yl]pyrrolidin-1-yl]-4,6-bis(trifluoromethyl)pyridine (34.7 mg) as a white solids.

### Example 26: ¹H NMR (300 MHz, DMSO-d₆): δ (ppm) 8.40 (s, 1H), 7.62-7.48 (m, 3H), 7.37-7.28 (m, 3H), 7.04 (s, 2H), 5.19 (s, 1H), 3.79-64 (m, 2H), 2.28-2.27 (m, 2H), 2.00-1.89 (m, 2H). m/z 456 (M+H⁺)

### Example 27: ¹H NMR (300 MHz, DMSO-d₆): δ (ppm) 8.40 (s, 1H), 7.62-7.48 (m, 3H), 7.37-7.28 (m, 3H), 7.04 (s, 2H), 5.19 (s, 1H), 3.79-64 (m, 2H), 2.28-2.27 (m, 2H), 2.00-1.89 (m, 2H). m/z 456 (M+H⁺)

### Example 28

### Synthesis of (S)-1-(4,6-bis(trifluoromethyl)pyridin-2-yl)-N,N-diphenylpyrrolidine-2-carboxamide

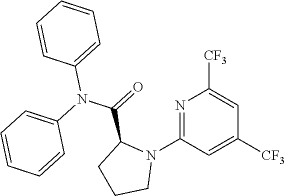 [SMILES: O=C([C@@H]1CCCN1c1cc(C(F)(F)F)cc(C(F)(F)F)n1)N(c1ccccc1)c1ccccc1]

### Step 1: Preparation of benzyl (S)-2-(diphenylcarbamoyl)pyrrolidine-1-carboxylate

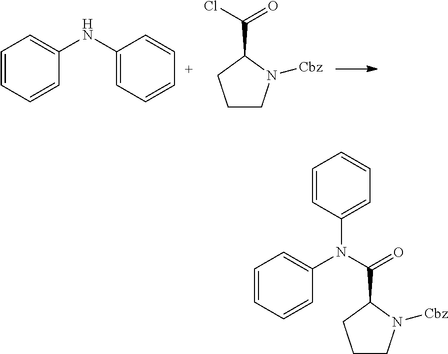

To a solution of (S)-benzyl 2-(chlorocarbonyl)pyrrolidine-1-carboxylate (500.00 mg, 2.006 mmol, 1.00 equiv) in DCM (5 mL) at room temperature was added diphenylamine (373.40 mg, 2.207 mmol, 1.10 equiv) followed by Et₃N (608.93 mg, 6.018 mmol, 3.00 equiv). The resulting mixture was stirred at room temperature for 4 h. The reaction mixture was diluted with water (15 mL) and the aqueous layer was extracted with DCM (3×5 mL). The combined organic extracts were dried over anhydrous sodium sulfate, filtered and concentrated under reduced pressure. The residue was purified by Prep-TLC (PE/EtOAc 20:1) to afford (S)-benzyl 2-(diphenylcarbamoyl)pyrrolidine-1-carboxylate (400 mg, 46%) as a dark brown oil.

### Step 2: Preparation of (S)—N,N-diphenylpyrrolidine-2-carboxamide

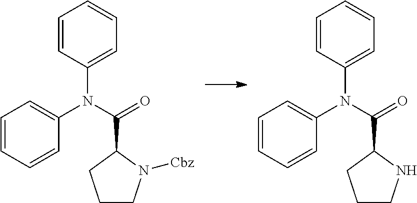 [SMILES: O=C([C@@H]1CCCN1C(=O)OCc1ccccc1)N(c1ccccc1)c1ccccc1 ; O=C([C@@H]1CCCN1)N(c1ccccc1)c1ccccc1]

To a solution of benzyl (2S)-2-(diphenylcarbamoyl)pyrrolidine-1-carboxylate (400.00 mg, 0.999 mmol, 1.00 equiv) in MeOH (5 mL) was added Pd/C (80 mg). The reaction mixture was evacuated and backfilled with nitrogen three times. The mixture was stirred at room temperature for 2 h under a hydrogen balloon. The solids were filtered off and the filter cake was washed with MeOH (3×20 mL). The combined filtrate was concentrated under reduced pressure. The residue was purified by Prep-TLC (PE/EtOAc 30:1) to afford (2S)—N,N-diphenylpyrrolidine-2-carboxamide (200 mg, 74%) as a yellow solid.

### Step 3: Preparation of(S)-1-(4,6-bis(trifluoromethyl)pyridin-2-yl)-N,N-diphenylpyrrolidine-2-carboxamide

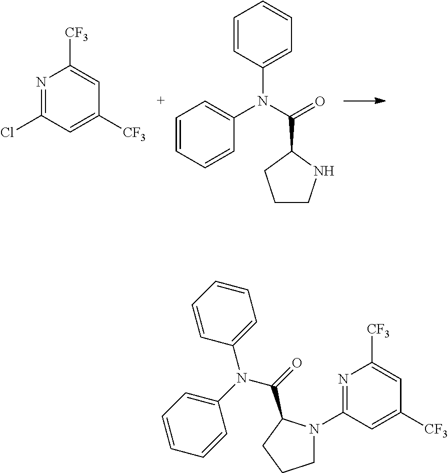 [SMILES: FC(F)(F)c1cc(Cl)nc(C(F)(F)F)c1 ; O=C([C@@H]1CCCN1)N(c1ccccc1)c1ccccc1 ; O=C([C@@H]1CCCN1c1cc(C(F)(F)F)cc(C(F)(F)F)n1)N(c1ccccc1)c1ccccc1]

The title compound was prepared using General Procedure C, employing (2S)—N,N-diphenylpyrrolidine-2-carboxamide and 2-chloro-4,6-bis(trifluoromethyl)pyridine as starting materials. The residue was purified by Prep-TLC (PE/EtOAc 30:1) to afford (S)-1-(4,6-bis(trifluoromethyl)pyridin-2-yl)-N,N-diphenylpyrrolidine-2-carboxamide (100 mg, 27%) as an off-white solid. ¹H NMR (300 MHz, DMSO-d₆): δ (ppm) 7.61-7.12 (m, 12H), 4.52-4.50 (m, 1H), 3.65-3.55 (m, 2H), 2.25-2.23 (m, 1H), 2.12-1.98 (m, 3H). m/z 480 (M+H⁺).

### Example 29

### Synthesis of (2S,3R)-1-(4,6-bis(trifluoromethyl)pyridin-2-yl)-N-(4-fluorophenyl)-3-hydroxy-N-methylpyrrolidine-2-carboxamide

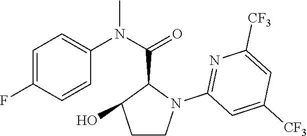 [SMILES: CN(C(=O)[C@@H]1[C@H](O)CCN1c1cc(C(F)(F)F)cc(C(F)(F)F)n1)c1ccc(F)cc1]

### Step 1: Preparation of tert-butyl (2S,3R)-2-((4-fluorophenyl)(methyl)carbamoyl)-3-hydroxypyrrolidine-1-carboxylate

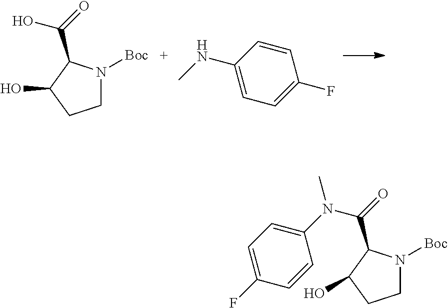

The title compound was prepared using General Procedure A, employing (2S,3R)-1-(tert-butoxycarbonyl)-3-hydroxypyrrolidine-2-carboxylic acid and 4-fluoro-N-methylaniline as starting materials. The residue was purified by reverse-phase flash chromatography (conditions: column—C18 silica gel; mobile phase—MeCN in water, 10% to 50% gradient over 10 min; detector—UV 220 nm) to afford tert-butyl (2S,3R)-2-[(4-fluorophenyl)(methyl)carbamoyl]-3-hydroxypyrrolidine-1-carboxylate (500 mg, 47%) as a yellow solid.

### Step 2: Preparation of (2S,3R)—N-(4-fluorophenyl)-3-hydroxy-N-methylpyrrolidine-2-carboxamide

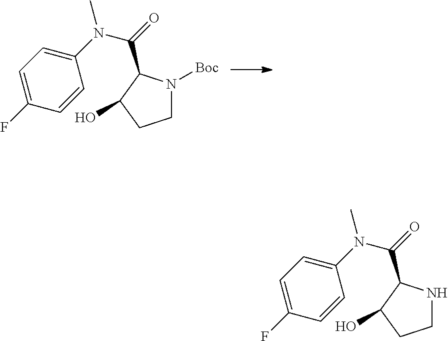 [SMILES: CN(C(=O)[C@@H]1[C@H](O)CCN1C(=O)OC(C)(C)C)c1ccc(F)cc1 ; CN(C(=O)[C@H]1NCC[C@H]1O)c1ccc(F)cc1]

The title compound was prepared using General Procedure B, employing tert-butyl (2S,3R)-2-[(4-fluorophenyl)(methyl)carbamoyl]-3-hydroxypyrrolidine-1-carboxylate as starting material. The reaction mixture was concentrated under reduced pressure to afford (2S,3R)—N-(4-fluorophenyl)-3-hydroxy-N-methylpyrrolidine-2-carboxamide (200 mg, 90%) as yellow oil.

### Step 3: Preparation of (2S,3R)-1-(4,6-bis(trifluoromethyl)pyridin-2-yl)-N-(4-fluorophenyl)-3-hydroxy-N-methypyrrolidine-2-carboxamide

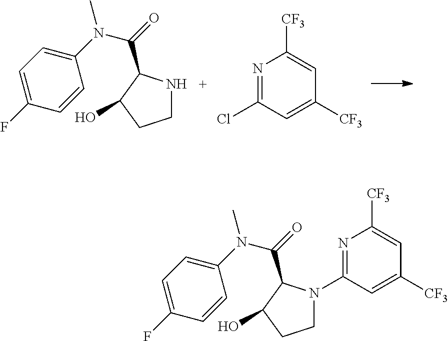 [SMILES: CN(C(=O)[C@H]1NCC[C@H]1O)c1ccc(F)cc1 ; FC(F)(F)c1cc(Cl)nc(C(F)(F)F)c1 ; CN(C(=O)[C@@H]1[C@H](O)CCN1c1cc(C(F)(F)F)cc(C(F)(F)F)n1)c1ccc(F)cc1]

The title compound was prepared using General Procedure C, employing (2S,3R)—N-(4-fluorophenyl)-3-hydroxy-N-methylpyrrolidine-2-carboxamide and 2-chloro-4,6-bis(trifluoromethyl)pyridine as starting materials. The residue was purified by Prep-TLC (PE/EtOAc 2:1) to afford (2S,3R)-1-[4,6-bis(trifluoromethyl)pyridin-2-yl]-N-(4-fluorophenyl)-3-hydroxy-N-methylpyrrolidine-2-carboxamide (177 mg, 51%) as a yellow solid. ¹H NMR (300 MHz, Methanol-d₄): δ (ppm) 7.66-7.62 (m, 2H), 7.28-7.19 (m, 2H), 7.11 (s, 1H), 7.02-6.95 (m, 1H), 4.72-4.70 (d, 1H), 4.37-4.28 (q, 1H), 3.80-3.72 (m, 1H), 3.54-3.46 (q, 1H), 3.26 (s, 3H), 2.27-2.19 (m, 2H). m/z 452 (M+H⁺).

### Example 30

### Synthesis of (S)-1-(4,6-bis(trifluoromethyl)pyridin-2-yl)-N-(4-fluorophenyl)-N-(oxetan-3-yl)pyrrolidine-2-carboxamide

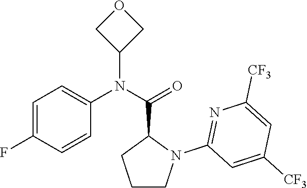 [SMILES: O=C([C@@H]1CCCN1c1cc(C(F)(F)F)cc(C(F)(F)F)n1)N(c1ccc(F)cc1)C1COC1]

### Step 1: Preparation of N-(4-fluorophenyl)oxetan-3-amine

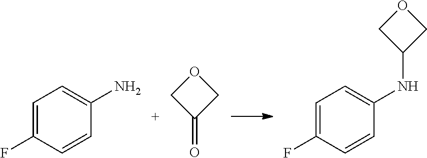 [SMILES: Nc1ccc(F)cc1 ; O=C1COC1 ; Fc1ccc(NC2COC2)cc1]

To a stirred solution of 4-fluoroaniline (2.00 g, 17.999 mmol, 1.00 equiv), 3-oxetanone (3.24 g, 44.997 mmol, 2.50 equiv) and HOAc (2.16 g, 35.997 mmol, 2.00 equiv) in MeOH (80.00 mL) at room temperature was added NaBH₃CN (2.26 g, 35.997 mmol, 2.00 equiv). The resulting mixture was stirred at room temperature for 6 h. The reaction mixture was diluted with water (60 mL) and neutralized to pH 7 with 1M aq. NaOH. The aqueous layer was extracted with CH₂Cl₂ (3×20 mL). The combined organic extracts were dried over anhydrous sodium sulfate, filtered and concentrated under reduced pressure. The residue was purified by silica gel column chromatography (eluent: 100% CH₂Cl₂) to afford N-(4-fluorophenyl)oxetan-3-amine (3.5 g, 116%) as a yellow solid.

### Step 2: Preparation of benzyl (S)-2-((4-fluorophenyl)(oxetan-3-yl)carbamoyl)pyrrolidine-1-carboxylate

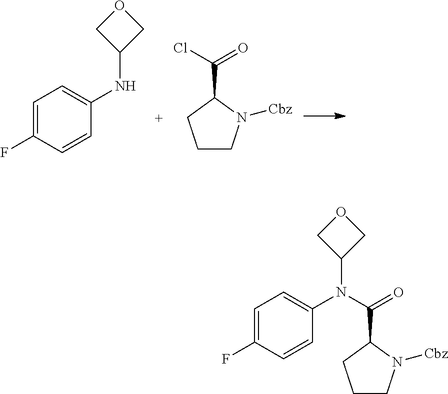

To a solution of N-(4-fluorophenyl)oxetan-3-amine (3.50 g, 20.935 mmol, 1.00 equiv) in DCM (35.00 mL) at room temperature was added benzyl (2S)-2-(carboxy)pyrrolidine-1-carboxylate (5.60 g, 20.935 mmol, 1.00 equiv). After stirring the reaction mixture for 3 h, the mixture was diluted with water (100 mL). The aqueous layer was extracted with CH₂Cl₂ (3×30 mL). The combined organic extracts were dried over anhydrous sodium sulfate, filtered and concentrated under reduced pressure. The residue was purified by silica gel column chromatography (eluent: 100% CH₂Cl₂) to afford benzyl (2S)-2-[(4-fluorophenyl)(oxetan-3-yl)carbamoyl]pyrrolidine-1-carboxylate (5.0 g, 60%) as a yellow solid.

### Step 3: Preparation of(S)—N-(4-fluorophenyl)-N-(oxetan-3-yl)pyrrolidine-2-carboxamide

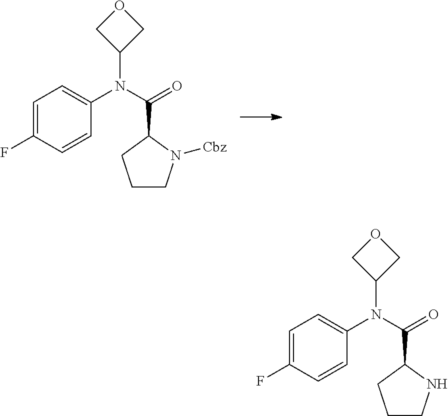 [SMILES: O=C([C@@H]1CCCN1C(=O)OCc1ccccc1)N(c1ccc(F)cc1)C1COC1 ; O=C([C@@H]1CCCN1)N(c1ccc(F)cc1)C1COC1]

To a solution of benzyl (2S)-2-[(4-fluorophenyl)(oxetan-3-yl)carbamoyl]pyrrolidine-1-carboxylate (300.00 mg, 0.753 mmol, 1.00 equiv) in MeOH (3.00 mL, 74.097 mmol, 98.41 equiv) at room temperature was added Pd/C (30.00 mg). The reaction mixture was degassed using house vacuum and stirred for 5 h under a hydrogen balloon. The solids were filtered off and the filter cake was washed with MeOH (3×5 mL). The combined filtrates were concentrated under reduced pressure. The residue was purified by Prep-TLC (CH₂Cl₂/MeOH 100:1) to afford (2S)—N-(4-fluorophenyl)-N-(oxetan-3-yl)pyrrolidine-2-carboxamide (190 mg, 96%) as a yellow solid.

### Step 4: Preparation of(S)-1-(4,6-bis(trifluoromethyl)pyridin-2-yl)-N-(4-fluorophenyl)-N-(oxetan-3-yl)pyrrolidine-2-carboxamide

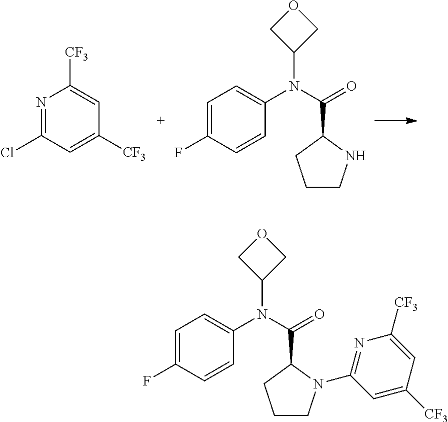 [SMILES: FC(F)(F)c1cc(Cl)nc(C(F)(F)F)c1 ; O=C([C@@H]1CCCN1)N(c1ccc(F)cc1)C1COC1 ; O=C([C@@H]1CCCN1c1cc(C(F)(F)F)cc(C(F)(F)F)n1)N(c1ccc(F)cc1)C1COC1]

The title compound was prepared using General Procedure C, employing (2S)—N-(4-fluorophenyl)-N-(oxetan-3-yl)pyrrolidine-2-carboxamide and 2-chloro-4,6-bis(trifluoromethyl)pyridine as starting materials. The residue was purified by Prep-TLC (PE/EtOAc 1:1) to afford (2S)-1-[4,6-bis(trifluoromethyl)pyridin-2-yl]-N-(4-fluorophenyl)-N-(oxetan-3-yl)pyrrolidine-2-carboxamide (114 mg, 37%) as a white semi-solid. ¹H NMR (300 MHz, DMSO-d₆): δ (ppm) 1.89-1.99 (m, 4H), 3.53-3.55 (m, 2H), 4.27-4.32 (m, 3H), 4.50-4.560 (m, 2H), 5.24-5.26 (m, 1H), 7.03 (s, 1H), 7.23 (s, 1H), 67.37-7.43 (m, 2H), 67.51-7.55 (m, 2H). m/z 478 (M+H⁺).

### Example 31

### Synthesis of (S)-1-(4,6-dimethylpyridin-2-yl)-N-(4-fluorophenyl)-N-methylpyrrolidine-2-carboxamide

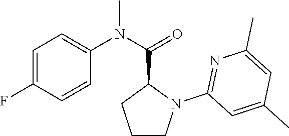 [SMILES: Cc1cc(C)nc(N2CCC[C@H]2C(=O)N(C)c2ccc(F)cc2)c1]

### Step 1: Preparation of tert-butyl (S)-2-((4-fluorophenyl)(methyl)carbamoyl)pyrrolidine-1-carboxylate

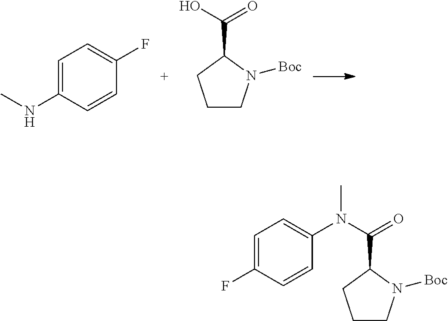

The title compound was prepared using General Procedure A, employing (2S)-1-(tert-butoxycarbonyl)pyrrolidine-2-carboxylic acid and 4-fluoro-N-methylaniline as starting materials. The residue was purified by Prep-TLC (PE/EtOAc 5:1) to afford tert-butyl (S)-2-((4-fluorophenyl)(methyl)carbamoyl)pyrrolidine-1-carboxylate (500 mg, 67% yield) as a dark green oil.

### Step 2: Preparation of (S)—N-(4-fluorophenyl)-N-methylpyrrolidine-2-carboxamide

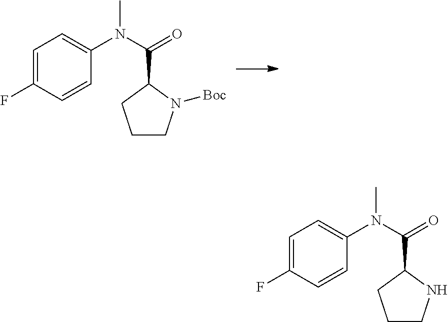 [SMILES: CN(C(=O)[C@@H]1CCCN1C(=O)OC(C)(C)C)c1ccc(F)cc1 ; CN(C(=O)[C@@H]1CCCN1)c1ccc(F)cc1]

The title compound was prepared using General Procedure B, employing tert-butyl (S)-2-((4-fluorophenyl)(methyl)carbamoyl)pyrrolidine-1-carboxylate as starting material. The crude product was used directly in the next step without further purification.

### Step 3: Preparation of (S)-1-(4,6-dimethylpyridin-2-yl)-N-(4-fluorophenyl)-N-methylpyrrolidine-2-carboxamide

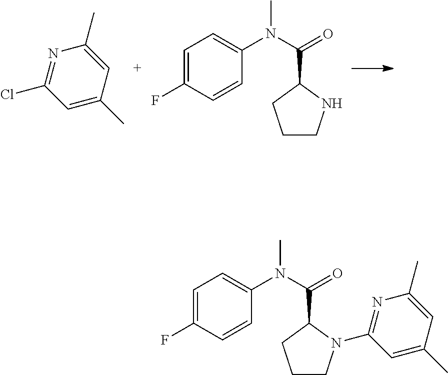

The title compound was prepared using General Procedure C, employing (S)—N-(4-fluorophenyl)-N-methylpyrrolidine-2-carboxamide and 2-chloro-4,6-dimethylpyridine as starting materials. The residue was purified by Prep-TLC (PE/EtOAc 2:1) to afford (S)-1-(4,6-dimethylpyridin-2-yl)-N-(4-fluorophenyl)-N-methylpyrrolidine-2-carboxamide (36.1 mg, 3.14% yield) as a light yellow solid. ¹H NMR (300 MHz, DMSO-d₆): δ (ppm) 7.62-7.60 (s, 2H), 7.38-7.32 (d, 2H), 6.27 (s, 1H), 6.02 (s, 1H), 4.34 (s, 1H), 3.48-3.41 (m, 1H), 3.36-3.31 (m, 1H), 3.13 (s, 3H), 2.30 (s, 3H), 2.16 (s, 3H), 2.08-2.01 (m, 1H), 2.00-1.88 (m, 3H). m/z 328 [M+H]⁺

### BIOLOGICAL EXAMPLES

### Biologic Example 1: Primer Extension Assay

The ability of the compounds of Formula (I) or Formula (II) to inhibit polymerase activity of Pol theta was determined using the primer extension assay as set forth below.

A mixture of 20 uL of Pol theta polymerase domain (residues 1819-2590) at a final concentration of 4 nM in assay buffer (20 m M TRIS, pH 7.80, 50 mM KCl, 10 mM MgCl₂, 1 mM DTT, 0.01% BSA, 0.01% Tween20) was added to test compounds (11-point dilution series of test compounds) except the low control wells without test compounds. The above enzyme and test compound inhibitor mixture was then incubated at room temperature for 15 min. An equal volume (20 μl) of dNTP substrate mixture (48 μM) and primed molecular beacon DNA (obtained by annealing template SEQ ID NO:2: (5′-CCTTCCTCCCGTGTCTTG-TACCTTCCCGTCA-GGAGGAAGG-3′) with 5′-TAMRA and 3′-BHQ and primer DNA (SEQ ID NO:3; 5′-GACGGGAAGG-3′) in 10 mM Tris-HCl pH 8.0, 100 mM NaCl buffer) (96 nM) in assay buffer was added to all the test wells. The inhibition activity was measured by monitoring the fluorescence change over 30 min at 535 nm upon excitation at 485 nm. The high control (DMSO with enzyme) with high fluorescence intensity represents no inhibition of polymerase reaction while the low control (DMSO with buffer) with low fluorescence intensity represents full inhibition of polymerase activity. Slope of the reaction progress curves were used to calculate the rate of polymerization. The rates were used to determine the percent inhibition using a four-parameter inhibition model to generate IC₅₀, Hill slope and max inhibition.

The IC₅₀ of the compounds in Table 1 above, as determined by the primer extension assay are provided in Table 2 below: 10 μM≥(+) >1 μM; 1 μM≥(++) >500 nM; 500 nM≥(+++) >200 nM; 200 nM≥(++++)

TABLE 2
Primer Extension Assay

| Cpd. No. | Primer extension Assay IC₅₀ |
|---|---|
| 1.001 | ++++ |
| 1.002 | + |
| 1.003 | ++++ |
| 1.004 | + |
| 1.005 | ++++ |
| 1.006 | ++++ |
| 1.007 | ++++ |
| 1.008 | ++++ |
| 1.009 | ++++ |
| 1.010 | ++++ |
| 1.011 | ++++ |
| 1.012 | ++++ |
| 1.013 | ++++ |
| 1.014 | ++++ |
| 1.015 | ++++ |
| 1.016 | ++++ |
| 1.017 | ++++ |
| 1.018 | ++++ |
| 1.019 | ++++ |
| 1.020 | ++++ |
| 1.021 | ND |
| 1.022 | ++++ |
| 1.023 | ND |
| 1.024 | ++++ |
| 1.025 | ND |
| 1.026 | ND |
| 1.027 | ND |
| 1.028 | ND |
| 1.029 | ND |
| 1.030 | ND |
| 1.031 | +++ |

ND = not determined

### Biologic Example 2: PPi Assay

The ability of the compounds of Formula (I) or Formula (II) to inhibit polymerase activity of Pol theta was determined using the PPi assay as set forth below.

A mixture of template DNA strand (SEQ ID NO:4: 5′ ATT ACT GAC CTC ATA CTT CTG CCC TTC CAT GTT CTG TGC CCT CCT TCC 3′) and primer DNA strand (SEQ ID NO:5: 5′ GGA AGG AGG GCA CAG AAC 3′) was annealed in 10 mM Tris-HCl pH 8.0, 50 NaCl buffer to form the primed DNA substrate. A 10-point dilution series of compounds were used in a 384 well format for the inhibition assay. Pol theta (residues 1819-2590) (2.8 nM) in assay buffer (20 mM Tris-HCl pH 7.8, 50 mM KCl, 10 mM MgCl₂, 1 mM DTT, 0.01% BSA, 0.01% Tween-20) was transferred to the test wells (10 uL), except for the low control wells. The plate was then incubated at room temperature for 15 mins. An equal volume (10 μL) of dNTP substrate (40 μM) and primed DNA substrate (800 nM) in assay buffer was added to all the test wells. 20 μL of PPi detection reagent (PPiLite inorganic pyrophosphate assay, Lonzo) was then added to all test wells. The plate was then centrifuged at 1000 rpm for 1 min. The reaction was monitored in a Tecan M1000 Pro plate reader in luminescence kinetic mode for 90 min. The high control (DMSO with enzyme) with high luminescence represents no inhibition of the polymerase reaction while the low control (DMSO with buffer) with low luminescence represents full inhibition of the polymerase activity. Slope of the reaction progress curves were used to calculate the rate of polymerization. The rates were used to determine the percent inhibition using a four-parameter inhibition model to generate IC₅₀, Hill slope, maximum inhibition, and minimum inhibition.

The IC₅₀ of the compounds in Table 1 above, as determined by the PPᵢ assay are provided in Table 3 below:

10 μM≥(+) >1 μM; 1 μM≥(++) >500 nM; 500 nM≥(+++) ≥200 nM; 200 nM≥(++++)

TABLE 3
PPi Assay

| Cpd. No. | PPi Assay IC₅₀ |
|---|---|
| 1.001 | ND |
| 1.002 | + |
| 1.003 | ++++ |
| 1.004 | ND |
| 1.005 | ND |
| 1.006 | + |
| 1.007 | ND |
| 1.008 | ND |
| 1.009 | ND |
| 1.010 | ND |
| 1.011 | ND |
| 1.012 | ND |
| 1.013 | ND |
| 1.014 | ND |
| 1.015 | ND |
| 1.016 | ND |
| 1.017 | ND |
| 1.018 | ND |
| 1.019 | ++++ |
| 1.020 | ++++ |
| 1.021 | ++++ |
| 1.022 | ++++ |
| 1.023 | ++ |
| 1.024 | ++++ |
| 1.025 | ++++ |
| 1.026 | ++++ |
| 1.027 | + |
| 1.028 | + |
| 1.029 | ++++ |
| 1.030 | ++++ |
| 1.031 | ND |

ND = not determined

Any patents or publications mentioned in this specification are indicative of the levels of those skilled in the art to which the inventive concept pertains. The patents and publications are herein incorporated by reference to the same extent as if each individual publication was specifically and individually indicated to be incorporated by reference. In case of conflict, the present specification, including definitions, will control.

One skilled in the art will readily appreciate that the present invention is well adapted to carry out the objects and obtain the ends and advantages mentioned, as well as those inherent therein. The present disclosures described herein are presently representative of particular embodiments, are exemplary, and are not intended as limitations on the scope of the invention. Changes therein and other uses will occur to those skilled in the art which are encompassed within the spirit of the invention as defined by the scope of the claims.

1. A compound of Formula (I): 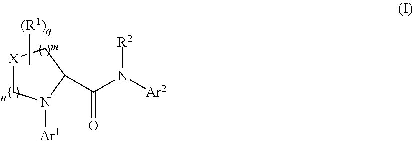
or a pharmaceutically acceptable salt thereof, wherein
X is selected from the group consisting of —CH₂—, —CHR¹—, —NR¹—, —NH—, and —O—;
m is an integer selected from the group consisting of 0, 1, and 2;
n is an integer selected from the group consisting of 0, 1, and 2;
provided that the sum of m and n is at least 1 and no more than 3;
q is an integer selected from the group consisting of 0, 1, and 2;
each R¹ is independently selected from the group consisting of C₁₋₈ alkyl, C₁₋₈ haloalkyl, —OR^{a}, —X¹—OR^{a}, —NR^{a}R^{b}, —X¹—NR^{a}R^{b}, —NR^{a}C(O)R^{b}—X¹—NR^{a}C(O)R^{b}, —C(O)NR^{a}R^{b}, —X¹—C(O)NR^{a}R^{b}, —C(O)R^{a}, —X¹—C(O)R^{a}, phenyl, and —X¹-phenyl, wherein X¹ is C₁₋₃ alkylene;
each R^{a} and R^{b} are independently selected from the group consisting of H, C₁₋₄ alkyl, and C₁₋₄ haloalkyl; and
phenyl is substituted with from 0 to 3 R^{c} moieties, each R^{c} is selected from the group consisting of C₁₋₈ alkyl, halo, C₁₋₈ haloalkyl, C₁₋₈ alkoxy, C₁₋₈ haloalkoxy, —OH, —X^{c}—OH, and cyano, wherein X^{c} is C₁₋₃ alkylene;
Ar¹ is 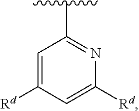
wherein each R^{d} is independently selected from the group consisting of C₁₋₈ alkyl, halo, C₁₋₈ haloalkyl, cyano, —OR^{e}, —NR^{e}R^{f}—NR^{e}C(O)R^{f}, and —C(O)NR^{e}R^{f}, wherein
each R^{e} and R^{f} are independently selected from the group consisting of H, C₁₋₆ alkyl, and C₁₋₆ haloalkyl;
R² is selected from the group consisting of C₁₋₈ alkyl, C₁₋₈ haloalkyl, C₃₋₆ cycloalkyl, phenyl, and 3- to 6-membered heterocycloalkyl having 1 to 3 heteroatom ring vertices independently selected from the group consisting of N, O, and S; and
Ar² is selected from the group consisting of phenyl and 6- to 10-membered heteroaryl having 1 to 4 heteroatom ring vertices independently selected from the group consisting of N, O, and S, wherein
Ar² is substituted with 0 to 4 R^{h} moieties, wherein each R^{h} is independently selected from the group consisting of C₁₋₈ alkyl, C₁₋₈ haloalkyl, halo, cyano, C₃₋₆ cycloalkyl, —ORⁱ, —NR^{j}R^{k}, —NR^{j}C(O)R^{k}, and —C(O)NR^{j}R^{k}, wherein
each Rⁱ is selected from the group consisting of H, C₁₋₆ alkyl, C₁₋₆ haloalkyl, and C₃₋₆ cycloalkyl; and
each R^{j} and R^{k} are independently selected from the group consisting of H, C₁₋₆ alkyl, and C₁₋₆ haloalkyl.

2. (canceled)

3. The compound of claim 1, having Formula (Ib-1) 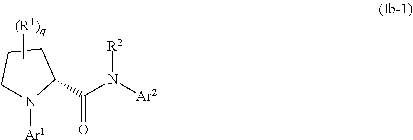
or a pharmaceutically acceptable salt thereof.

4. The compound of claim 1, having a formula (Ib-1i), or (Ib-1ii) 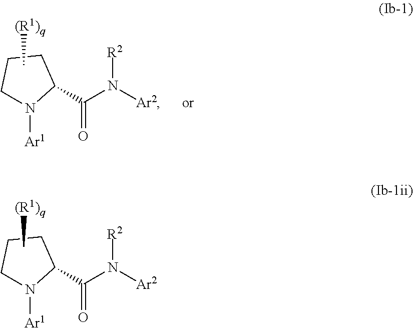
or a pharmaceutically acceptable salt thereof.

5. The compound of claim 1, having Formula (Ic) 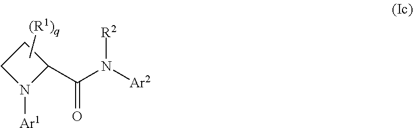
or a pharmaceutically acceptable salt thereof.

6-8. (canceled)

9. The compound of claim 1, having Formula (Id) 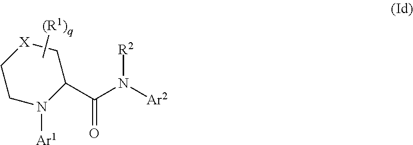
or a pharmaceutically acceptable salt thereof.

10-12. (canceled)

13. The compound of claim 1, wherein X is —CH₂—.

14. The compound of claim 1, wherein X is —NH—.

15-16. (canceled)

17. The compound of claim 1, where q is 1.

18-19. (canceled)

20. The compound of claim 1, wherein each R¹ is independently selected from the group consisting of C₁₋₈ alkyl, C₁₋₈ haloalkyl, —OR^{a}, —NR^{a}R^{b}, —NR^{a}C(O)R^{b}, —C(O)NR^{a}R^{b}, and —C(O)R^{a}, wherein
each R^{a} and R^{b} are independently selected from the group consisting of H, C₁₋₄ alkyl, and C₁₋₄ haloalkyl.

21-26. (canceled)

27. The compound of any one of claim 1, wherein each R^{d} is independently selected from the group consisting of C₁₋₈ alkyl, halo, C₁₋₈ haloalkyl, and cyano.

28-29. (canceled)

30. The compound of claim 1, wherein R² is selected from the group consisting of C₁₋₂ alkyl, C₁₋₂ haloalkyl, cycloproyl, and oxetanyl.

31. The compound of claim 1, wherein Ar² is phenyl substituted with 0 to 3 R^{h} moieties.

32. (canceled)

33. The compound of claim 1, wherein Ar² is 6- to 10-membered heteroaryl having 1 to 4 heteroatom ring vertices independently selected from the group consisting of N, O, and S, and the 6- to 10-membered heteroaryl is substituted with 0 to 3 R^{h} moieties.

34-41. (canceled)

42. A compound of Formula (II): 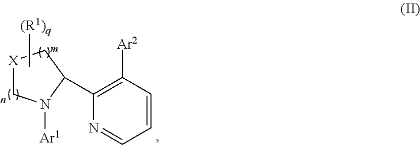
a pharmaceutically acceptable salt thereof, wherein
X is selected from the group consisting of —CH₂—, —CHR¹—, —NR¹—, —NH—, and —O—;
m is an integer selected from the group consisting of 0, 1, and 2;
n is an integer selected from the group consisting of 0, 1, and 2;
provided that the sum of m and n is at least 1 and no more than 3;
q is an integer selected from the group consisting of 0, 1, and 2;
each R¹ is independently selected from the group consisting of C₁₋₈ alkyl, C₁₋₈ haloalkyl, —OR^{a}, —X¹—OR^{a}, —NR^{a}R^{b}, —X¹—NR^{a}R^{b}, —NR^{a}C(O)R^{b}, —X¹—NR^{a}C(O)R^{b}, —C(O)NR^{a}R^{b}, —X¹—C(O)NR^{a}R^{b}, —C(O)R^{a}, —X¹—C(O)R^{a}, phenyl, and —X¹-phenyl, wherein
X¹ is C₁₋₃ alkylene;
each R^{a} and R^{b} are independently selected from the group consisting of H, C₁₋₄ alkyl, and C₁₋₄ haloalkyl; and
phenyl is substituted with from 0 to 3 R^{c} moieties, each R^{c} is selected from the group consisting of C₁₋₈ alkyl, halo, C₁₋₈ haloalkyl, C₁₋₈ alkoxy, C₁₋₈ haloalkoxy, —OH, —X^{c}—OH, and cyano, wherein X^{c} is C₁₋₃ alkylene;
Ar¹ is selected from the group consisting of phenyl and 6- to 10-membered heteroaryl having 1 to 4 heteroatom ring vertices independently selected from the group consisting of N, O, and S, wherein
Ar¹ is substituted with 0 to 4 R^{d} moieties, wherein each R^{d} is independently selected from the group consisting of C₁₋₈ alkyl, halo, C₁₋₈ haloalkyl, cyano, —OR^{e}, —NR^{e}R^{f}—NR^{e}C(O)R^{f}, and —C(O)NR^{e}R^{f}, wherein
each R^{e} and R^{f} are independently selected from the group consisting of H, C₁₋₆ alkyl, and C₁₋₆ haloalkyl;
Ar² is selected from the group consisting of phenyl and 6- to 10-membered heteroaryl having 1 to 4 heteroatom ring vertices independently selected from the group consisting of N, O, and S, wherein
Ar² is substituted with 0 to 4 R^{h} moieties, wherein each R^{h} is independently selected from the group consisting of C₁₋₈ alkyl, C₁₋₈ haloalkyl, halo, cyano, C₃₋₆ cycloalkyl, —ORⁱ, —NR^{j}R^{k}, —NR^{j}C(O)R^{k}, and —C(O)NR^{j}R^{k}, wherein
each Rⁱ is selected from the group consisting of H, C₁₋₆ alkyl, C₁₋₆ haloalkyl, and C₃₋₆ cycloalkyl; and
each R^{j} and R^{k} are independently selected from the group consisting of H, C₁₋₆ alkyl, and C₁₋₆ haloalkyl.

43. A compound of claim 42, having Formula (IIa): 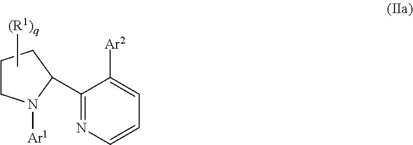
or a pharmaceutically acceptable salt thereof.

44. A compound of claim 42, having Formula (IIb): 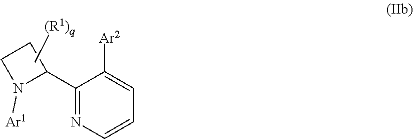
or a pharmaceutically acceptable salt thereof.

45. The compound of claim 42, having Formula (IIc) 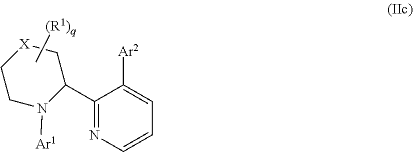
or a pharmaceutically acceptable salt thereof.

46-71. (canceled)

72. A pharmaceutical composition comprising a compound of claim 1, or a pharmaceutically acceptable thereof and at least one pharmaceutically acceptable excipient.

73. A method for treating a disease characterized by overexpression of Polθ in a patient comprising administering to the patient a therapeutically effective amount of the compound of claim 1.

74. (canceled)

75. A method of treating a homologous recombinant (HR) deficient cancer in a patient comprising administering to the patient a therapeutically effective amount of the compound of claim 1.

76. (canceled)

77. A method for treating a cancer in a patient, wherein the cancer is characterized by a reduction or absence of BRCA gene expression, the absence of the BRCA gene, or reduced function of BRCA protein, comprising administering to the patient a therapeutically effective amount of claim 1.

78. The method of claim 77, wherein the cancer is lymphoma, soft tissue sarcoma, rhabdoid tumor, multiple myeloma, uterus caner, gastric cancer, peripheral nervous system cancer, rhabdomyosarcoma, bone cancer, colorectal cancer, mesothelioma, breast cancer, ovarian cancer, lung cancer, central nervous system cancer, urinary tract cancer, upper aerodigestive track cancer, leukemia, kidney cancer, skin cancer, esophagus cancer, or pancreas cancer.

79-91. (canceled)


SEQUENCE LISTING (5 sequences)

SEQ ID NO: 1 - DNA, 40 bp, Artificial
  Synthetic template sequence
ccttcctcccgtgtcttgtaccttcccgtcaggaggaagg

SEQ ID NO: 2 - DNA, 40 bp, Artificial; identical to SEQ ID NO: 1
  Synthetic template sequence
  misc_feature (1)..(1): 5′-TAMRA label
  misc_feature (40)..(40): 3′-BHQ label

SEQ ID NO: 3 - DNA, 10 bp, Artificial
  Synthetic primer sequence
gacgggaagg

SEQ ID NO: 4 - DNA, 48 bp, Artificial
  Synthetic template sequence
attactgacctcatacttctgcccttccatgttctgtgccctccttcc

SEQ ID NO: 5 - DNA, 18 bp, Artificial
  Synthetic primer sequence
ggaaggagggcacagaac